(19) United States
(12) Patent Application Publication
(10) Pub. No.: US 2025/0295610 A1
(43) Pub. Date: Sep. 25, 2025

(54) NEUROPROTECTIVE AGENTS FOR USE IN THE TREATMENT OF OPTIC NEUROPATHIES

(71) Applicant: The Board of Trustees of the Leland Stanford Junior University, Stanford, CA (US)

(72) Inventors: Yang Hu, Menlo Park, CA (US); Wei Chen, Stanford, CA (US)

(21) Appl. No.: 18/714,931

(22) PCT Filed: Nov. 28, 2022
(86) PCT No.: PCT/US2022/080499

§ 371 Date: May 30, 2024

Related U.S. Application Data
(60) Provisional application No. 63/284,424, filed on Nov. 30, 2021.

(51) Int. Cl. A61K 31/135; A61K 31/4545; A61K 31/553; A61K 31/7088; A61K 38/46; A61K 48/00; A61P 27/06

(52) U.S. Cl. CPC A61K 31/135; A61K 31/4545; A61K 31/553; A61K 31/7088; A61K 38/465; A61K 48/0033; A61K 48/0066; A61P 27/06

(57) ABSTRACT

The present disclosure provides compositions and methods for treating a mammalian subject for an ON neuropathies and/or reducing or ameliorating degeneration of axons and/or soma of RGCs. Aspects of the composition include a neuroprotective agent and a pharmaceutically acceptable excipient, where the neuroprotective agent inhibits HRH1 activity. A variety of ON neuropathies may be treated by practicing the methods, including retinal ganglion cell degeneration, glaucoma, optic neuritis, ON traumatic injury and other ON-related diseases.

### CROSS REFERENCE TO OTHER APPLICATIONS

This application claims the benefit of PCT Application No. PCT/US2022/080499, filed Nov. 28, 2022, which claims benefit of U.S. provisional application No. 63/284,424, filed Nov. 30, 2021, the contents of which are hereby incorporated by reference in its entirety.

### GOVERNMENT RIGHTS

This invention was made with Government support under contract EY023295 awarded by the National Institutes of Health. The Government has certain rights in the invention.

### INCORPORATION BY REFERENCE OF SEQUENCE LISTING PROVIDED AS A TEXT FILE

A Sequence Listing is provided herewith as a Sequence Listing XML, “S21-382_STAN-1910” created on Dec. 19, 2024, and having a size of 35,783 bytes. The contents of the Sequence Listing XML are incorporated by reference herein in their entirety.

### INTRODUCTION

The most common cause of irreversible blindness, glaucoma, will affect an estimated 3% of the world population over 40 years old by 2040 (more than 100 million people), which will impose a multi-billion dollar economic burden on society. Glaucoma is characterized by optic neuropathy with optic nerve (ON) degeneration followed by progressive retinal ganglion cell (RGC) death. The only available treatments act by reducing intraocular pressure (IOP), a risk factor associated with glaucoma. However, IOP reduction fails to completely prevent the progression of glaucomatous neurodegeneration, indicating the urgent need for innovative neuroprotection therapies.

ON injury induces neuronal endoplasmic reticulum (ER) stress in RGCs, suggesting a detrimental role of RGC-specific ER stress in glaucoma. When the protein or calcium homeostasis of the ER is adversely altered, cells experience ER stress and activate three signaling pathways initiated by three ER-resident stress-sensing proteins: inositol-requiring protein-1 (IRE1α), activating transcription factor-6 (ATF6) and protein kinase RNA-like ER kinase (PERK), together called the unfolded protein response (UPR).

IRE1α, a bi-functional enzyme that contains both a Ser/Thr kinase domain and an endoribonuclease (RNase) domain, mediates the splicing of X-box binding protein 1 (XBP-1) mRNA to generate an active (spliced) form of the transcription factor, XBP-1s. The IRE1α-XBP-1s pathway targets genes that increase ER protein-folding capacity and facilitate degradation of misfolded proteins. On the other hand, IRE1α kinase activity also activates pro-apoptotic c-Jun kinase (JNK), which contributes to Bax-dependent IRE1α-induced apoptosis. ATF6 is a transcription factor that is truncated and thereby activated by ER stress to control the expression of a group of UPR target genes. PERK phosphorylates and inactivates eukaryotic translation initiation factor 2α (eIF2α) to attenuate global cap-dependent mRNA translation and thereby reduce protein load on the ER.

A small subset of genes have specific upstream open reading frame (uORF) motifs in their mRNAs that can overcome this suppression and are more efficiently translated under the ER stress condition, including activating transcription factor 4 (ATF4) and C/EBP homologous protein (CHOP). ATF4 also induces the expression of, and forms heterodimers with, CHOP to cause cell death by upregulating protein synthesis and inducing oxidative stress. CHOP is a well-known pro-apoptotic transcription factor that mediates ER stress-induced cell death by downregulating anti-apoptotic Bcl2, upregulating pro-apoptotic BH-3 only molecules Bim and PUMA, increasing expression of death receptor 5 (DR5) and caspase 8 cleavage. Chronic ER stress with prolonged PERK-eIF2α-ATF4-CHOP signaling has been associated with many acute and chronic neurodegenerative diseases; genetic manipulation and small molecular modulators of this pathway have proven to be beneficial in animal models of various neurodegenerative diseases.

Provided herein are methods and neuroprotective agents for the treatment of optic neuropathies.

### SUMMARY

Compositions and methods for treating a mammalian subject for optic nerve (ON) neuropathies, and/or reducing or ameliorating degeneration of axons and/or soma of retinal ganglion cells (RGCs) by administering an effective dose of a histamine receptor H1 (HRH1) antagonist. HRH1 Antagonists are demonstrated herein to be neuroprotective agents that can provide significant RGC and ON neuroprotection and preservation of visual functions in response to injury or stress. In some embodiments an HRH1 antagonist is a tricyclic compound. In other embodiments an HRH1 antagonist is a genetic construct encoding an HRH1 inhibitor. Aspects of the disclosure include formulations of an HRH1 antagonist and a pharmaceutically acceptable excipient that are suitable for delivery to the eye, and which may provide for sustained release delivery to the eye. A variety of ON neuropathies may be treated by practicing the methods, including without limitation retinal ganglion cell degeneration, glaucoma, optic neuritis, ON traumatic injury and other ON-related diseases. The subject may be diagnosed with an ON neuropathy prior to treatment.

As described in the present disclosure, high-throughput screening identified novel neuroprotective agents based on their ability to inhibit C/EBP homologous protein (CHOP) based endoplasmic reticulum (ER) stress. Neuroprotective agents blocked ER stress-induced CHOP activity, suppressed the unfolded protein response (UPR) pathway, and significantly protected retinal ganglion cells (RGCs), optic nerve cells (ON), and visual functions in disease models of glaucoma and traumatic injury. Neuroprotective effects of neuroprotective agents were achieved through pharmacological inhibition of histamine receptor H1 (HRH1) mediated Ca²⁺ release from the ER. Genetic inhibition of HRH1 was shown to provide neuroprotective effects similar to that of pharmacological inhibition.

The neuroprotective effects of an HRH1 antagonist can include a range of outcomes. For instance, neuroprotective effects may include, without limitation, a reduction in neuronal cell body death, a reduction in neuronal axon death, an improvement in visual acuity when compared to the absence of treatment, an increase in ganglion cell complex size relative to the absence of treatment, and the like.

In an embodiment, a method is provided for treating a mammalian subject for optic nerve (ON) neuropathies, and/or reducing or ameliorating degeneration of axons and/or soma of retinal ganglion cells (RGCs) by administering an effective dose of a tricyclic compound that inhibits HRH1. In some embodiments, the neuroprotective agent is selected from amoxapine, desipramine, desloratadine, trifluoperazine, clomipramine, amitriptyline, quetiapine, olanzapine, maprotiline, doxepin, loxapine, integrated stress response inhibitor (ISRIB), and norquetiapine. In some embodiments the neuroprotective agent is one of amoxapine, desloratadine and maprotiline. In some embodiments the neuroprotective agent is maprotiline.

In some embodiments a formulation is provided, comprising a tricyclic compound that inhibits HRH1. In some embodiments, the neuroprotective agent is selected from amoxapine, desipramine, desloratadine, trifluoperazine, clomipramine, amitriptyline, quetiapine, olanzapine, maprotiline, doxepin, loxapine, integrated stress response inhibitor (ISRIB), and norquetiapine. In some embodiments the neuroprotective agent is one of amoxapine, desloratadine and maprotiline. In some embodiments the neuroprotective agent is maprotiline. The formulation may be provided in a unit dose for delivery to the eye. The formulation may be provided for intra-vitreal injection. The formulation may be provided for sustained release to the eye. In some aspects, provided herein is a method of inducing neuroprotection/increasing survival/promoting functional recovery of RGC somata and axons, comprising intravitreally administering the composition into a mammalian subject experiencing or at risk of an ON axonopathy. In some embodiments of the method, the ON neuropathy is retinal ganglion cell degeneration, including glaucoma, optic neuritis, ON traumatic injury and other ON-related diseases

In an embodiment, a method is provided for treating a mammalian subject for optic nerve (ON) neuropathies, and/or reducing or ameliorating degeneration of axons and/or soma of retinal ganglion cells (RGCs) by administering an effective dose of a therapeutic gene therapy viral vector, comprising a murine γ-synuclein promoter, or functional fragment thereof, that promotes expression of a transgene specifically in RGCs, said promoter in operable linkage with an expression cassette encoding the transgene, wherein the expressed transgene inhibits activity of an expression product of an endogenous HRH1 gene. In some embodiments, the therapeutic gene therapy vector is an AAV virus comprising a therapeutic sequence. In some embodiments, the therapeutic vector comprises a CRISPR/Cas9 system and at least one guide RNA (gRNA) directed to a HRH1 gene.

In some embodiments a formulation is provided, comprising an effective dose of a therapeutic gene therapy viral vector, comprising a murine γ-synuclein promoter, or functional fragment thereof, that promotes expression of a transgene specifically in RGCs, said promoter in operable linkage with an expression cassette encoding the transgene, wherein the expressed transgene inhibits activity of an expression product of an endogenous HRH1 gene. The formulation may be provided in a unit dose for delivery to the eye. The formulation may be provided for intra-vitreal injection. The formulation may be provided for sustained release to the eye. In some aspects, provided herein is a method of inducing neuroprotection/increasing survival/promoting functional recovery of RGC somata and axons, comprising intravitreally administering the composition into a mammalian subject experiencing or at risk of an ON axonopathy. In some embodiments of the method, the ON neuropathy is retinal ganglion cell degeneration, including glaucoma, optic neuritis, ON traumatic injury and other ON-related diseases

Aspects of the disclosure include administering a formulation disclosed herein to treat the subject for the ON neuropathy either intravitreally or systemically. When the formulation is administered, it may be administered at a time that is dependent on the type of ON neuropathy being treated. For instance, if the ON neuropathy is the result of traumatic injury, the composition may be administered within hours, 1 day, 2 days, 3 days, 4 days, 5 days, 6 days, one week, two weeks or more than two weeks after traumatic injury.

In some embodiments, the composition of the present disclosure may be administered with a secondary treatment modality that is used to further treat the optic neuropathy. If the optic neuropathy is glaucoma, a number of different secondary treatment modalities may be administered. For instance, secondary treatment modalities for glaucoma may include, without limitation, prostaglandins (e.g. latanoprost, travoprost, tafluprost, or bimatoprost), rho kinase inhibitors (e.g. netarsudil), nitric oxides (e.g. latanoprostene bunod), miotic or cholinergic agents (e.g. pilocarpine), alpha-adrenergic agonists (e.g. apraclonidine or brimonidine), beta blockers (e.g. betaxolol or timolol), carbonic anhydrase inhibitors (e.g. dorzolamide or brinzolamide), etc.

### BRIEF DESCRIPTION OF THE FIGURES

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIGS. 1A-1G. Cell-based HTS to identify small molecule inhibitors of the CHOP branch of UPR. a The schematic HTS pipeline employed to identify small molecules that inhibit the CHOP pathway of the ER stress with the HEK293T reporter cell line expressing CHOP promoter-driven luciferase (CHOP-Luc) and downstream in vitro and in vivo assays for further characterization. b Plot showing the percentage inhibition of CHOP-Luc signals of each tested library compound in the presence of Tm/Tg (1 μM), 24 hours after exposure. “Hit” threshold is set at >30% inhibition (red dotted line) but <100% inhibition. c Heatmap of dose-dependent responses of 89 “hits” in CHOP-Luc inhibition. Compounds a-e with similar chemical structure are marked with red lines. d Relative (to DMSO) CHOP-Luc activities of 12 compounds and 2 control compounds GSK2606414 and ISRIB at 10 UM in the presence of Tm/Tg (1 μM), 24 hours after exposure. Data are presented as means±s.e.m, n=3 independent replicates, ****: P<0.0001, ***: P<0.001, *: P<0.05, one-way ANOVA with Dunnett's multiple comparisons test. e Chemical structures of amoxapine (Amo), desloratadine (Desl) and maprotiline (Map). f IC₅₀ calculated with nonlinear regression through dose-dependent fits of CHOP-Luc activities (relative to DMSO) of individual compounds at indicated concentration in the presence of Tm/Tg (1 μM), 24 hours after exposure. Data are presented as means±s.e.m, n=3 independent replicates. g Cell viability assay of Amo, Desl and Map on HEK293T cells. Data are presented as means±s.e.m, n=3 independent replicates.

FIGS. 2A-2E. Amo, Desl and Map inhibit the signaling cascades downstream of the three UPR pathways. a Immunoblot of HEK293T cells showing the effects of the three compounds on the PERK-CHOP pathway. Quantification of phosphorylated PERK (p-PERK), ATF4 and CHOP 24 hours after DMSO, Amo, Desl or Map (10 UM) treatment in the presence of Tm/Tg (1 μM), relative to control (without Tm/Tg). b Immunoblot showing the effects of the three compounds on the ATF6 pathway. Quantification of total ATF6 protein level 24 hours after DMSO, Amo, Desl or Map treatment in the presence of Tm/Tg, relative to control (without Tm/Tg). c RT-PCR showing the mRNA levels of un-spliced and spliced forms of XBP-1 (XBP-1u and XBP-1s). Quantification of XBP-1s mRNA level 24 hours after DMSO, Amo, Desl or Map treatment in the presence of Tm/Tg, relative to control (without Tm/Tg). d Immunoblot showing the effects of the three compounds on the IRE1α-XBP-1/JNK pathway. Quantification of XBP-1s, phosphorylated JNK and IRE1α 24 hours after DMSO, Amo, Desl or Map treatment in the presence of Tm/Tg, relative to control (without Tm/Tg). e qPCR mRNA measurement of the UPR genes in HEK293T cells treated for 12 hours with DMSO, Amo, Desl, Map (10 UM) in the presence of Tm/Tg (1 μM), relative to control (without Tm/Tg). All data in this figure are presented as means±s.e.m, n=3 independent replicates, *: P<0.05, **: P<0.01, *** P<0.001, **** P<0.0001, one-way ANOVA with Dunnett's multiple comparisons test.

FIGS. 3A-31. In vivo application of the three hit compounds inhibits ONC-induced ER stress in RGCs. a Schematic depicting the mouse ONC model and the timeline of ER stress and neuroprotection evaluation. For compound treatment, each eye received intravitreal injection with 2 μl of 2 mM test compounds once and intraperitoneal (i.p.) injection daily (15 mg/kg). (b, d, f, h) Immunohistochemistry analysis showing the levels of CHOP (b), p-eIF2α (d), ATF4 (f) and p-JNK (h) in GCL (ganglion cell layer) of retina sections at 3dpc. (c, e, g, i) Quantification of corresponding fluorescence intensities of CHOP (b), p-eIF2α (d), ATF4 (f) and p-JNK (h) in GCL. Data are presented as means±s.e.m, n=3 mice, *: P<0.05, **: P<0.01, *** P<0.001, **** P<0.0001, one-way ANOVA with Dunnett's multiple comparisons test.

FIGS. 4A-4F. In vivo application of the three hit compounds significantly protects RGC somata and axons after traumatic ONC injury. a Representative OCT images of mouse retina in living animals at 14dpc. GCC: ganglion cell complex, including RNFL, GCL and IPL layers; indicated as double end red arrows. b Quantification of GCC thickness measured by OCT at 14dpc, represented as percentage of GCC thickness in the ONC eyes compared to the sham contralateral control eyes. n=10 mice for DMSO, Amo, Desl, and Map; n=6 mice for ISRIB. c Representative confocal images of peripheral flat-mounted retinas showing surviving RBPMS+ (green) RGCs at 14dpc. d Quantification of surviving RGCs at 14dpc, represented as percentage of ONC eyes compared to the sham contralateral control eyes. n=10 mice for DMSO, Amo, Desl, and Map; n=6 mice for ISRIB. e Light microscope images of semi-thin transverse sections of ON with PPD staining at 14dpc. f Quantification of surviving RGC axons in ON at 14dpc, represented as percentage of crushed ONs compared to the sham contralateral control ONs. n=10 mice for DMSO, Amo, Desl, and Map; n=6 mice for ISRIB. All data in this figure are presented as means±s.e.m, **** P<0.0001 *** P<0.001, ** P<0.01, *P<0.05, ns: no significance, one-way ANOVA with Dunnett's multiple comparisons test to compare each treatment to DMSO group. For compound treatment, each eye received intravitreal injection with 2 μl of 2 mM test compounds once and intraperitoneal (i.p.) injection daily (15 mg/kg). Control groups received the same volume of DMSO as vehicle control.

FIGS. 5A-5G. Systemic administration of Map significantly protects RGC somata and axons and preserves visual function in a mouse glaucoma model. a IOP measurements at 3wpi. SOHU: intracameral injection of SO to induce ocular hypertension. n=10 mice. b Representative OCT images of mouse retina in living animals at 3wpi. GCC is indicated as double end red arrows. c Quantification of GCC thickness measured by OCT at 3wpi, represented as percentage of GCC thickness in the SOHU eyes compared to the sham contralateral control eyes. n=9 mice. d Upper panel: representative confocal images of wholemounts of the entire retinas. Middle panel: representative confocal images of flat-mounted peripheral retinas showing surviving RBPMS+ (red) RGCs at 3wpi. Lower panel: representative light microscope images of semi-thin transverse sections of ON with PPD staining at 3wpi. e Quantification of surviving RGC somata in wholemount retinas and axons in ON sections at 3wpi, represented as percentage in SOHU eyes compared to the sham contralateral control eyes. n=10 mice. f Left: representative wave forms of PERG at baseline and 3wpi. Right: quantification of P1-N2 amplitude of PERG at 3wpi, represented as percentage in glaucomatous eyes compared to the sham contralateral control eyes. n=8 mice. g Left: illustration of OKR measurement of mouse. Right: visual acuity measured by OKR at 3wpi, represented as percentage of cycles/degree value in the glaucomatous eyes compared to the sham contralateral control eyes. n=9 mice. All data in this figure are presented as means±s.e.m, *** P<0.001, **** P<0.0001, one-way ANOVA with Dunnett's multiple comparisons test. For compound treatment, each eye received retrobulbar injection twice on day 0 and day 10 with 50 μl of 2 mM maprotiline, and daily i.p. injection with maprotiline (15 mg/kg) for 3 weeks after SO injection. Control groups received the same volume of DMSO as vehicle control.

FIGS. 6A-6F. The effects on ER stress modulation of HRH1, the common target of the three hit compounds. a Relative CHOP-Luc activities of the three compounds at indicated concentrations in the presence of Tm/Tg (1 μM), with or without HRH1 overexpression (OE), relative to DMSO, 24 hours after exposure. n=3 independent replicates. *: P<0.05, **: P<0.01, with a two-tailed unpaired Student's t-test. b Immunoblot of HEK293T cells showing the CRISPR-mediated HRH1 KD and quantification of HRH1 protein levels. n=3 independent replicates. **: P<0.01, with a two-tailed unpaired Student's t-test. c Relative CHOP-Luc (n=5 independent replicates) activities in response to Tm/Tg at indicated concentrations, with or without HRH1-KD, 24 hours after exposure. d Immunoblot of HEK293T cells showing the protein levels of ATF4 and CHOP with or without HRH1 inhibition. Quantification of relative protein levels. Data are presented as means±s.e.m, n=3 independent replicates, **** P<0.0001, *** P<0.001, ns: no significance, one-way ANOVA with Dunnett's multiple comparisons test. (e, f) Left: immunohistochemistry analysis showing the levels of CHOP (e), and ATF4 (f) in GCL of retina sections at 3dpc. Right: quantification of corresponding fluorescence intensities/area in GCL. Data are presented as means±s.e.m, n=4 mice, *: P<0.05, ** P<0.01, one-way ANOVA with Dunnett's multiple comparisons test.

FIGS. 7A-7L. AAV-mediated in vivo CRISPR KD of HRH1 in RGCs significantly protects RGCs and ONs and preserves visual function in two optic neuropathy models. a Schematic illustration of the timelines of AAV injection and evaluation of neuroprotection in two optic neuropathy models. b Representative OCT images of mouse retina in at 14dpc. GCC is indicated as double end red arrows. c Quantification of GCC thickness measured by OCT at 14dpc. AAV-CL, n=7 mice; HRH1-KD, n=5 mice. d Upper panel: representative confocal images of the wholemount retinas; Lower panel: representative light microscope images of semi-thin transverse sections of ON with PPD staining at 14dpc. e Quantification of surviving RGC somata in wholemount retinas and axons in ON sections at 14dpc. n=10 mice for AAV-CL, Amo; n=5 mice for HRH1-KD. f Representative OCT images of mouse retina in living mice with SOHU glaucoma at 3wpi. GCC is indicated as double end red arrows. g Quantification of GCC thickness measured by OCT at 3wpi. n=9 mice. h Representative confocal images of the whole retina and enlarged peripheral retina showing surviving RBPMS+ (red) RGCs at 3wpi; and representative light microscope images of semi-thin transverse sections of ON with PPD staining. i Quantification of surviving RGC somata in wholemount retinas and axons in ON sections at 3wpi, represented as percentage of SOHU eyes compared to the sham contralateral control eyes. n=9. j Visual acuity measured by OKR at 3wpi. n=9 mice. k Left: representative wave forms of PERG at baseline and 3wpi. Right: quantification of P1-N2 amplitude of PERG at 3wpi. n=9 mice. All data in this figure are presented as means±s.e.m, ** P<0.01, *** P<0.001, **** P<0.0001, with a two-tailed unpaired Student t-test.

FIGS. 8A-8G. Map effectively inhibits axon injury-induced intracellular Ca^{2 +} influx and ER Ca²⁺ release. a jGCaMP7s expression in HEK293T cells as an indicator of cytoplasmic Ca^{2 +} levels. b Quantification of jGCaMP7s fluorescence intensity, 0.5 hours after DMSO or Map treatment in the presence of Tm/Tg, represented as fold changes to DMSO-treated control cells. n=9 independent replicates. c Cytoplasmic Ca^{2 +} influx in HEK293T cells immediately after Tm/Tg (1 μM) treatment, n=3 independent replicates. d In vivo retina Ca^{2 +} imaging by SLO in living animals expressing jGCaMP7s in RGCs shows cytoplasmic Ca^{2 +} influx in RGCs induced by axon injury. e Quantification of relative intra-RGC Ca^{2 +} levels, represented as fold changes to the baseline fluorescence intensity of DMSO-treated retina. n=5 mice. f Ex vivo measurement of ER Ca^{2 +} retention in RGCs expressing D4ER after ONC. The D4ER FRET ratio value (Citrine/ECFP) reflects the steady-state ER Ca^{2 +} concentration. g Quantification of ER Ca^{2 +} levels in RGCs expressing D4ER, represented as FRET ratios. n=5 mice. All data in this figure are presented as means±s.e.m, ** P<0.01, *** P<0.001, **** P<0.0001, one-way ANOVA with Dunnett's multiple comparisons test.

FIGS. 9A-9F CHOP-Luc cell-based assay. Relative CHOP-Luc activities at indicated concentrations of thapsigargin (Tg) (a) tunicamycin (b), and Tm/Tg (c) relative to baseline, 24 hours after exposure. n=4 independent replicates. d Chemical structures of hit compounds a-e and 9 additional analog compounds with similar structures. e Relative CHOP-Luc activities of individual tested compounds at 20 μM in the presence of Tm/Tg (1 μM) relative to DMSO, 24 hours after exposure. n=3 independent replicates. f Relative CHOP-Luc activities of individual tested compounds at 10 UM, compared to Tm/Tg at 1 μM, relative to baseline, 24 hours after exposure. n=8 independent replicates. All data in this figure are presented as means±s.e.m, *P <0.05, ** P<0.01, *** P<0.001, **** P<0.0001, ns: no significance, one-way ANOVA with Dunnett's multiple comparisons test.

FIGS. 10A-10D. ER stress inhibition in RGCs and ONs by the three compounds. (a, b) Representative confocal images of flat-mount retinas showing CHOP or p-JNK signals in RGCs at 3dpc. c Immunoblot of mouse ONs showing protein levels of ER stress molecules in ONs at 3dpc. d Quantification of relative protein levels of ER stress molecules in ONs at 3dpc. Data are presented as means±s.e.m, n=3 independent replicates, **** P<0.0001, *** P<0.001, ** P<0.01, *P<0.05, one-way ANOVA with Dunnett's multiple comparisons test.

FIGS. 11A-11B. Significant RGC axon protection by the three hit compounds in ONC revealed by TEM. a Representative TEM images of transverse sections of ON (4,000× magnification). b Quantification of surviving RGC axons in ONs at 14dpc, represented as percentage of crushed ONs compared to the sham contralateral control ONs. Data are presented as means±s.e.m, n=5 mice, **** P<0.0001, *** P<0.001, ** P<0.01, one-way ANOVA with Dunnett's multiple comparisons test.

FIGS. 12A-12C. In vivo application of three hit compounds inhibits SOHU-induced ER stress in RGCs. a LC-MS analysis of retina exposure of Map after i.p. injection at 6 and 24 hours. (b, c) Immunohistochemistry results showing the levels of CHOP (b) and ATF4 (c) in GCL of retina sections at 1wpi in SOHU mouse eyes. Quantification of corresponding fluorescence intensity/area in GCL. Data are presented as means±s.e.m, n=4 independent replicates, *P<0.05, ** P<0.01, *** P<0.001, ns: no significance, one-way ANOVA with Dunnett's multiple comparisons test.

FIGS. 13A-13D. Significant RGC axon protection by systemic administration of Map in SOHU glaucoma model. a Representative TEM images of transverse sections of ON (4,000× magnification). b Quantification of surviving RGC axons in ONs at 3wpi, represented as percentage of SOHU ONs compared to the sham contralateral control ONs. Data are presented as means±s.e.m, n=5 mice, **** P<0.0001, one-way ANOVA with Dunnett's multiple comparisons test. c Confocal images of wholemount ONs with anterograde CTB tracing at 3wpi. Scalebar d Quantification of the percentage of CTB-labeled area to total ON area. n=3 mice. Data are presented as means±s.e.m, **** P<0.0001, *** P<0.001, ** P<0.01, one-way ANOVA with Dunnett's multiple comparisons test.

FIGS. 14A-14J. The effect of HRH1 on XBP-1 splicing. a Schematic depicting the vectors for HRH1 OE and CRISPR-mediated KD, with SEQ ID NO:27 and SEQ ID NO:28. b Schematic illustrating the XBP-1-Luc stable cell line to report IRE1α activity and XBP-1 splicing. (c-e) Relative XBP-1s-Luc activities at indicated concentrations of Tg, Tm, or Tm/Tg in relative to baseline, 24 hours after exposure. Data are presented as means±s.e.m, n=4 independent replicates. f Relative XBP-1s-Luc activity of Map at indicated concentrations in the presence of Tm/Tg (1 μM), with or without HRH1-OE, relative to DMSO, 24 hours after exposure. n=3 independent replicates. g Relative XBP-1s-Luc activities at indicated concentrations of Amo, Desl and Map with or without HRH1-OE, in the presence of Tm/Tg (1 μM), relative to DMSO, 24 hours after exposure. Data are presented as means±s.e.m, n=3 independent replicates, with a two-tailed unpaired Student's t-test. h Relative XBP-1s-Luc (n=3 independent replicates) activities in response to Tm/Tg at indicated concentrations, with or without HRH1-KD, 24 hours after exposure. i Immunoblot of HEK293T cells showing the protein levels of ER stress molecules in the IRE1α and ATF6 pathways. Quantification of relative protein levels. Data are presented as means±s.e.m, n=3 independent replicates, *** P<0.001, ** P<0.01, *P<0.05, ns: no significance, one-way ANOVA with Dunnett's multiple comparisons test. j RT-PCR showing the mRNA levels of un-spliced and spliced forms of XBP-1 (XBP-1u and XBP-1s).

FIGS. 15A-15E. AAV-mediated Cas9 and gRNA expression in RGCs and HRH1 levels in RGCs. a Schematic illustration of the AAV vectors expressing Cas9 under the mSncg promoter and a pair of gRNAs targeting mouse HRH1 (mHRH1). b Confocal images of flat-mounted retinas showing AAV-mediated in vivo expression of HA-tagged Cas9, gRNAs-EGFP and RBPMS+RGCs. c Fluorescent in situ hybridization of mHRH1 mRNA in GCL of mouse retina sections. d Representative confocal images of retina section showing HRH1 protein levels in naïve and ONC (3dpc) mouse RGCs. e Representative confocal images of retina section showing HRH1 protein levels in naïve and SOHU glaucoma (1wpi) mouse RGCs. Data are presented as means±s.e.m, n=4 mice, *P<0.05, ns: no significance, with a two-tailed unpaired Student's t-test.

FIGS. 16A-16F. No detectable long term safety issues with systemic Map administration or locally CRISPR-mediated HRH1 KD in RGCs. a Representative images of wholemount retina, ON section and in vivo OCT, after Map treatment for 1 month or AAV-mediated CRISPR KD of HRH1 for 3 months. b Quantification of survival of RGC somata and axons and GCC thickness, represented as percentage of tested eyes compared to the sham contralateral control eyes. n=5 mice. All data are presented as means±s.e.m, ns: no significance, one-way ANOVA with Dunnett's multiple comparisons test. c Representative images of retina sections labeled with markers for RGC (RBPMS), T cell (CD3) and macrophage (Iba1). d Representative images of ON longitudinal sections labeled with axon marker (Tuj1), and cell markers for T cell (CD3) and macrophage (Iba1). e Quantification of inflammatory cell infiltration. n=4 independent replicates. All the data are presented as means±s.e.m, ns: no significance, one-way ANOVA with Dunnett's multiple comparisons test. f H&E staining of retina and ON sections after Map treatment for 1 month or AAV-mediated CRISPR KD of HRH1 for 3 months.

FIGS. 17A-17D. ER stress modulation and neuroprotection with pharmacological inhibitors of downstream HRH1 signaling. a Schematic depicting the HRH1 signaling molecules and corresponding chemical inhibitors. (b, c) ER stress reporter assays with CHOP-Luc (b) and XBP1-Luc cells (c) in the presence of Tm&Tg with or without U-73122, 2-AP and Go 6983 treatments. n=4 independent replicates for Ctr, 2-Ap, U73122 and GO6983; n=11 independent replicates for DMSO; mean±s.e.m; *** P<0.001, ** P<0.01, *P<0.05, ns: no significance, one-way ANOVA with Dunnett's multiple comparisons test. (d) Upper panel: representative confocal images of the wholemount retinas, showing surviving RBPMS+ (Red) RGCs at 14dpc; Lower panel: representative light microscope images of semi-thin transverse sections of ON with PPD staining at 14dpc. Quantification of surviving RGC somata in wholemount retinas and axons in ON sections at 14dpc, represented as percentage of ONC eyes compared to the sham contralateral control eyes. n=6 mice; data are presented as mean±s.e.m, *** P<0.001, **** P <0.0001, with a two-tailed unpaired Student's t test.

FIGS. 18A-18C. AAV-mSncg promoter-mediated jGCaMP7s and D4ER expression in mouse RGCs in vivo. a Representative confocal images of wholemount retina expressing jGCaMP7s 2 weeks after AAV-mSncg-jGCaMP7s intravitreal injection and colocalization of RGC marker RBPMS with jGCaMP7s analyzed by Image J. b In vivo retina imaging by SLO in living animals expressing jGCaMP7s in RGCs showing cytoplasmic Ca^{2 +} influx in RGCs induced by ONC. c Representative confocal images of wholemount retina expressing D4ER 2 weeks after AAV-mSncg-D4ER intravitreal injection and colocalization of RGC marker RBPMS with D4ER analyzed by Image J.

### DEFINITIONS

The terms “treatment,” “treating,” “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. The term “treatment” encompasses any treatment of a disease in a mammal, particularly a human, and includes: (a) preventing the disease and/or symptom(s) from occurring in a subject who may be predisposed to the disease or symptom(s) but has not yet been diagnosed as having it; (b) inhibiting the disease and/or symptom(s), i.e., arresting development of a disease and/or the associated symptoms; or (c) relieving the disease and the associated symptom(s), i.e., causing regression of the disease and/or symptom(s). Those in need of treatment can include those already inflicted (e.g., those with optic neuropathies) as well as those in which prevention is desired (e.g., those with increased susceptibility to optic neuropathies; those with optic neuropathies; those suspected of having optic neuropathies; etc.).

The terms “recipient,” “individual,” “subject,” “host,” and “patient” are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, camels, etc. In some embodiments, the mammal is human.

The terms “specific binding,” “specifically binds,” and the like, refer to non-covalent or covalent preferential binding to a molecule relative to other molecules or moieties in a solution or reaction mixture (e.g., a neuroprotective agent specifically binds to a HRH1 relative to other available polypeptides or small molecules). In some embodiments, the affinity of one molecule for another molecule to which it specifically binds is characterized by a K_{D} (dissociation constant) of 10⁻⁵ M or less (e.g., 10⁻⁶ M or less, 10⁻⁷ M or less, 10⁻⁸ M or less, 10⁻⁹ M or less, 10⁻¹⁰ M or less, 10⁻¹¹ M or less, 10⁻¹² M or less, 10⁻¹³ M or less, 10⁻¹⁴ M or less, 10⁻¹⁵ M or less, or 10⁻¹⁶ M or less). “Affinity” refers to the strength of binding, increased binding affinity being correlated with a lower K_{D}.

The terms “co-administration” and “in combination with” include the administration of two or more therapeutic agents either simultaneously, concurrently or sequentially within no specific time limits. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to (e.g., minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks) after the administration of a second therapeutic agent.

The term “sample” as used herein relates to a material or mixture of materials, typically, although not necessarily, in fluid, i.e., aqueous, form, containing one or more components of interest. Samples may be derived from a variety of sources such as from food stuffs, environmental materials, a biological sample or solid, such as tissue or fluid isolated from an individual, including but not limited to, for example, plasma, serum, spinal fluid, semen, lymph fluid, the external sections of the skin, respiratory, intestinal, and genitourinary tracts, tears, saliva, milk, blood cells, tumors, organs, and also samples of in vitro cell culture constituents (including but not limited to conditioned medium resulting from the growth of cells in cell culture medium, putatively virally infected cells, recombinant cells, and cell components). In certain embodiments of the method, the sample includes a cell. In some instances of the method, the cell is in vitro. In some instances of the method, the cell is in vivo.

The terms “polynucleotide” and “nucleic acid,” used interchangeably herein, refer to a polymeric form of nucleotides of any length, either ribonucleotides or deoxynucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The terms “polynucleotide” and “nucleic acid” should be understood to include, as applicable to the embodiment being described, single-stranded (such as sense or antisense) and double-stranded polynucleotides.

The terms “polypeptide,” “peptide,” and “protein”, are used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like. The term “polypeptide” includes lipoproteins, glycoproteins, and the like.

A “host cell,” as used herein, denotes an in vivo or in vitro eukaryotic cell, or a cell from a multicellular organism (e.g., a cell line) cultured as a unicellular entity, which eukaryotic cells can be, or have been, used as recipients for a nucleic acid (e.g., an expression vector), and include the progeny of the original cell which has been genetically modified by the nucleic acid. It is understood that the progeny of a single cell may not necessarily be completely identical in morphology or in genomic or total DNA complement as the original parent, due to natural, accidental, or deliberate mutation. A “recombinant host cell” (also referred to as a “genetically modified host cell”) is a host cell into which has been introduced a heterologous nucleic acid, e.g., an expression vector, a guide RNA, a donor DNA template, and the like. For example, a subject eukaryotic host cell is a genetically modified eukaryotic host cell, by virtue of introduction into a suitable eukaryotic host cell of a heterologous nucleic acid, e.g., an exogenous nucleic acid that is foreign to the eukaryotic host cell, or a recombinant nucleic acid that is not normally found in the eukaryotic host cell.

As used herein, “CRISPER/Cas9 system” typically includes a polynucleotide sequence (which may, together, be referred to as an expression cassette, or one or more gRNAs may be separately encoded and referred to as a cassette), wherein the polynucleotide sequence encodes the Cas9 nuclease alone, or also encodes one or more guide RNAs (gRNAs) as well as the Cas9 nuclease. In some instances, the expression cassette encoding the CRISPER/Cas9 system is referred to as a “transgene.”

The term ‘neuroprotective’ as used herein refers to the ability to protect neurons or their axons or synapses in the central or peripheral nervous system from damage or death. Many different types of insult can lead to neuronal damage or death, for example: metabolic stress caused by hypoxia, hypoglycemia, diabetes, loss of ionic homeostasis or other deleterious process, physical injury of neurons, exposure to toxic agents and numerous diseases affecting the nervous system including inherited disorders. The presence of an agent that is neuroprotective enables a neuron to remain viable upon exposure to insults that would otherwise cause a loss of functional integrity in an unprotected neuron.

The term ‘injury’ as used herein refers to damage inflicted on the neuron, whether in the cell body or in axonal or dendritic processes. This can be a physical injury in the conventional sense i.e. traumatic injury to the brain, spinal cord or peripheral nerves caused by an external force applied to a subject. Other damaging external factors are for example environmental toxins such as mercury and other heavy metals, pesticides and solvents. Alternatively, injury can result from an insult to the neuron originating from within the subject, for example: reduced oxygen and energy supply as in ischemic stroke and diabetic neuropathy, autoimmune attack as in multiple sclerosis or oxidative stress and free-radical generation as is believed to be important in amyotrophic lateral sclerosis. Injury is also used here to refer to any defect in the mechanism of axonal transport.

### DETAILED DESCRIPTION

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference in their entirety, as if each individual publication or patent were specifically and individually indicated to be incorporated by reference, and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the compositions and methods have been or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

Endoplasmic reticulum (ER) stress and its downstream unfolded protein response (UPR) pathways play a critical role in both neuronal cell body and axon degeneration. In 2012, Hu et al. first reported that ON injury induces ER stress in RGCs (Hu et al., 2012). ER stress activates a complex cascade of reactions, in general called the unfolded protein response (UPR) (Walter and Ron, 2011; Wang and Kaufman, 2016). Striking neuroprotection has been accomplished by manipulating downstream signaling molecules individually or combined of ER stress/UPR, including C/EBP homologous protein (CHOP), X-Box-Binding Protein 1 (XBP-1), eukaryotic translation initiation factor 2 alpha (eIF2α), Activating Transcription Factor 6 (ATF6) and Activating Transcription Factor 4 (ATF4) (R. Sano, J.C. Reed, (2013), Biochimica et Biophysica Acta 1833:3460-3470). Of special interest, it was found that modulating these molecules could achieve neuroprotection in three optic neuropathy models (Hu et al., 2012; Yang et al., 2016a; Huang et al., 2017), indicating that ER stress is a common mechanism for neurodegeneration. Thus, targeting ER stress/UPR molecules have considerable therapeutic neuroprotective potential in neural injury/diseases associated neurodegeneration.

### Tricyclic Pharmacologic Formulations

High-throughput screening as disclosed herein identified novel neuroprotective agents based on their ability to modulate C/EBP homologous protein (CHOP) based endoplasmic reticulum (ER) stress. Neuroprotective agents blocked ER stress-induced CHOP expression, suppressed the unfolded protein response (UPR) pathway, and significantly protected retinal ganglion cells (RGCs), optic nerve cells (ON), and visual functions in disease models of glaucoma and traumatic injury. Neuroprotective effects of neuroprotective agents were achieved through pharmacological inhibition of histamine receptor H1 (HRH1) mediated Ca^{2 +} release from the ER.

In some embodiments, the neuroprotective agent is an antagonist or inhibitor of HRH1. In some such embodiments a subject is treated for glaucoma by administering an effective dose of an HRH1 antagonist for a period of time effective to provide neuroprotection. In some embodiments, the neuroprotective agent is a tricyclic compound. In some embodiments, the neuroprotective agent is selected from amoxapine, desipramine, desloratadine, trifluoperazine, clomipramine, amitriptyline, quetiapine, olanzapine, maprotiline, doxepin, loxapine, integrated stress response inhibitor (ISRIB), and norquetiapine. In some embodiments the neuroprotective agent is one of amoxapine, desloratadine and maprotiline. In some embodiments the neuroprotective agent is maprotiline. In some embodiments maprotiline is used and formulated for treatment of glaucoma.

Tricyclic compounds, and particularly maprotiline, provides well-characterized safety profiles, pharmacokinetics and pharmacodynamics, including penetration of blood-brain barrier. In some embodiments the formulation is delivered to the eye, including without limitation, intravitreal injection, ocular drops, sustained release implants for ocular use, and the like. In some embodiments a sustained release formulation for ocular delivery is provided.

The formulation can be administered by any suitable means, including ocular, intra-vitreal, oral, parenteral, etc. Parenteral infusions include intramuscular, intravenous (bolus or slow drip), intraarterial, intraperitoneal, intrathecal or subcutaneous administration. An agent can be administered in any manner which is medically acceptable. Sustained release administration is also specifically included in the disclosure, by such means as depot injections or erodible implants.

As noted above, an agent can be formulated with an a pharmaceutically acceptable carrier (one or more organic or inorganic ingredients, natural or synthetic, with which a subject agent is combined to facilitate its application). A suitable carrier includes sterile saline although other aqueous and non-aqueous isotonic sterile solutions and sterile suspensions known to be pharmaceutically acceptable are known to those of ordinary skill in the art. An “effective amount” refers to that amount which is capable of ameliorating or delaying progression of the diseased, degenerative or damaged condition. An effective amount can be determined on an individual basis and will be based, in part, on consideration of the symptoms to be treated and results sought. An effective amount can be determined by one of ordinary skill in the art employing such factors and using no more than routine experimentation.

In some embodiments an effective dose of a tricyclic agent, e.g. maprotiline, is from 0.1 μg/retina to about 1 mg/retina or more, e.g. from about 0.5 μg, about 1 μg, about 5 μg, about 10 μg, about 25 μg, about 50 μg, about 75 μg, about 100 μg, about 250 μg, about 500 μg, about 750 μg, about 1 mg. In some embodiments the agent is provided in a sustained release formulation that delivers the agent of a period of over about 1 day, 2 days, 3 days, 4 days, 5 days, 6 days, 7 days, 8 days, 9 days, 10 days, 11 days, 12 days, 13 days, 14 days, or more, e.g. over 2 weeks, 3 weeks 4 weeks, or more.

The volume in intravitreal injection, per injection, may be not more than about 500 μl, not more than about 200 μl, not more than about 100 μl, and may be from about 1 μl to about 200 μl, from about 5 μl to about 100 μl, from about 25 μl to about 100 μl, and may be around 50 μl.

Formulations suitable for injection can be administered by an intravitreal, intraocular, or other route of administration, e.g., injection into the retina.

An agent can be administered as a pharmaceutical composition comprising a pharmaceutically acceptable excipient. The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

As used herein, compounds which are “commercially available” may be obtained from commercial sources including but not limited to Acros Organics (Pittsburgh PA), Aldrich Chemical (Milwaukee WI, including Sigma Chemical and Fluka), Apin Chemicals Ltd. (Milton Park UK), Avocado Research (Lancashire U.K.), BDH Inc. (Toronto, Canada), Bionet (Cornwall, U.K.), Chemservice Inc. (West Chester PA), Crescent Chemical Co. (Hauppauge NY), Eastman Organic Chemicals, Eastman Kodak Company (Rochester NY), Fisher Scientific Co. (Pittsburgh PA), Fisons Chemicals (Leicestershire UK), Frontier Scientific (Logan UT), ICN Biomedicals, Inc. (Costa Mesa CA), Key Organics (Cornwall U.K.), Lancaster Synthesis (Windham NH), Maybridge Chemical Co. Ltd. (Cornwall U.K.), Parish Chemical Co. (Orem UT), Pfaltz & Bauer, Inc. (Waterbury CN), Polyorganix (Houston TX), Pierce Chemical Co. (Rockford IL), Riedel de Haen AG (Hannover, Germany), Spectrum Quality Product, Inc. (New Brunswick, NJ), TCI America (Portland OR), Trans World Chemicals, Inc. (Rockville MD), Wako Chemicals USA, Inc. (Richmond VA), Novabiochem and Argonaut Technology.

Compounds can also be made by methods known to one of ordinary skill in the art. As used herein, “methods known to one of ordinary skill in the art” may be identified though various reference books and databases. Suitable reference books and treatises that detail the synthesis of reactants useful in the preparation of compounds of the present invention, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Specific and analogous reactants may also be identified through the indices of known chemicals prepared by the Chemical Abstract Service of the American Chemical Society, which are available in most public and university libraries, as well as through on-line databases (the American Chemical Society, Washington, D.C. may be contacted for more details). Chemicals that are known but not commercially available in catalogs may be prepared by custom chemical synthesis houses, where many of the standard chemical supply houses (e.g., those listed above) provide custom synthesis services.

In some embodiments, pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes).

A carrier may bear the agents in a variety of ways, including covalent bonding either directly or via a linker group, and non-covalent associations. Suitable covalent-bond carriers include proteins such as albumins, peptides, and polysaccharides such as aminodextran, each of which have multiple sites for the attachment of moieties. The nature of the carrier can be either soluble or insoluble for purposes of the invention.

Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyidimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). Formulations to be used for in vivo administration must be sterile. This is readily accomplished by filtration through sterile filtration membranes.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Compositions can be prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249:1527, 1990 and Hanes, Advanced Drug Delivery Reviews 28:97-119, 1997. The agents of this invention can be administered in the form of a depot injection or implant preparation which can be formulated in such a manner as to permit a sustained or pulsatile release of the active ingredient. The pharmaceutical compositions are generally formulated as sterile, substantially isotonic and in full compliance with all Good Manufacturing Practice (GMP) regulations of the U.S. Food and Drug Administration.

Toxicity of the active agents can be determined by standard pharmaceutical procedures in cell cultures or experimental animals, e.g., by determining the LD50 (the dose lethal to 50% of the population) or the LD100 (the dose lethal to 100% of the population). The dose ratio between toxic and therapeutic effect is the therapeutic index. The data obtained from these cell culture assays and animal studies can be used in further optimizing and/or defining a therapeutic dosage range and/or a sub-therapeutic dosage range (e.g., for use in humans). The exact formulation, route of administration and dosage can be chosen by the individual physician in view of the patient's condition.

### Gene Therapy Formulations

Deletion of CHOP, one of the critical UPR genes that mediates ER stress-induced apoptosis in many disease models (Zinszner et al., 1998; Oyadomari et al., 2002; Silva et al., 2005; Song et al., 2008), preserves the structure and function of both RGC somata and axons in experimental ON crush, glaucoma and experimental autoimmune encephalomyelitis (EAE)/optic neuritis (Hu et al., 2012; Yang et al., 2016a; Huang et al., 2017). Because neuronal soma and axon degenerations are active autonomous processes with distinct molecular mechanisms (Calkins and Horner, 2012; Wang et al., 2012; Howell et al., 2013; Conforti et al., 2014; Geden and Deshmukh, 2016; Gerdts et al., 2016), targeting both provides better functional recovery than targeting either alone. Sterile alpha and TIR motif-containing protein 1 (SARM1) has recently been found to be critical for axon degeneration, and its deletion reported to preserve the integrity of injured axons (Osterloh et al., 2012; Gerdts et al., 2013).

Gene therapy utilizing a viral vehicle to deliver genetic material into cells is a promising way to directly target pathogenetic molecules (Keeler et al., 2017). The retina is an advantageous target for gene therapy due to its easy access, confined non-systemic localization, partial immune privilege, and well-established definitive functional readouts (Ratican et al., 2018). The Clustered Regularly Interspaced Short Palindromic Repeats (CRISPR) and CRISPR-associated protein (Cas) system, which bacteria use to edit foreign viral gene intrusion, has been adapted for efficient gene editing of the mammalian genome (Cong et al., 2013; Jinek et al., 2013; Mali et al., 2013). Mature retinal neurons are post-mitotic cells, in which specific gene deletion can be achieved by CRISPR/Cas9-mediated double-stranded DNA breaks and nonhomologous end joining (NEJ) (Wang et al., 2014b; Bakondi et al., 2016). The simplicity and specificity of the RNA-guided CRISPR/Cas9 endonuclease system has revolutionized gene therapy by enabling precise, efficient and even multiplex gene editing in mammalian cells (Cong et al., 2013; Jinek et al., 2013; Mali et al., 2013). Taking advantage of these powerful genetic tools to develop neuroprotective treatments for optic neuropathy is a scientifically important and clinically urgent undertaking.

AAV gene therapy. Utilizing a viral vehicle to deliver genetic material into cells allows direct targeting of pathogenic molecules and restoration of function. The retina is an advantageous target for gene therapy due to its easy access, confined non-systemic localization, partial immune privilege, and well-established definitive functional readouts. The success of adeno-associated virus (AAV)-mediated gene replacement in treating inherited retinal disease makes RGC-specific therapy with AAV a promising gene therapy strategy for optic neuropathies. Because AAV is non-pathogenic and cannot reproduce itself without helper viruses, it has served as a primary vehicle for gene therapy. It is a single-stranded DNA virus that stably and efficiently infects a wide variety of cells in multiple tissues. AAV2, the best-characterized AAV serotype, efficiently infects RGCs in retina after intravitreal injection.

In some embodiments, the vector is a recombinant adeno-associated virus (AAV) vector. AAV vectors are DNA viruses of relatively small size that can integrate, in a stable and site specific manner, into the genome of the cells that they infect. They are able to infect a wide spectrum of cells without inducing any effects on cellular growth, morphology or differentiation, and they do not appear to be involved in human pathologies. The AAV genome has been cloned, sequenced and characterized. It encompasses approximately 4700 bases and contains an inverted terminal repeat (ITR) region of approximately 145 bases at each end, which serves as an origin of replication for the virus. The remainder of the genome is divided into two essential regions that carry the encapsidation functions: the left-hand part of the genome, that contains the rep gene involved in viral replication and expression of the viral genes; and the right-hand part of the genome, that contains the cap gene encoding the capsid proteins of the virus.

The application of AAV as a vector for gene therapy has been rapidly developed in recent years. Wild-type AAV can infect, with a comparatively high titer, dividing or non-dividing cells, or tissues of mammal, including human, and also can integrate into in human cells at specific site (on the long arm of chromosome 19) (Kotin et al, Proc. Natl. Acad. Sci. U.S.A., 1990. 87:2211-2215; Samulski et al, EMBO J., 1991. 10:3941-3950 the disclosures of which are hereby incorporated by reference herein in their entireties). AAV vector without the rep and cap genes loses specificity of site-specific integration, but may still mediate long-term stable expression of exogenous genes. AAV vector exists in cells in two forms, wherein one is episomic outside of the chromosome; another is integrated into the chromosome, with the former as the major form. Moreover, AAV has not been found to be associated with any human disease, nor any change of biological characteristics arising from the integration has been observed. There are sixteen serotypes of AAV reported in literature, respectively named AAV1, AAV2, AAV3, AAV4, AAV5, AAV6, AAV7, AAV8, AAV9, AAV10, AAV11, AAV12, AAV13, AAV14, AAV15, and AAV16, wherein AAV5 is originally isolated from humans (Bantel-Schaal, and H. zur Hausen. Virology, 1984. 134:52-63), while AAV1-4 and AAV6 are all found in the study of adenovirus (Ursula Bantel-Schaal, Hajo Delius and Harald zur Hausen. J. Viral., 1999. 73:939-947).

AAV vectors may be prepared using any convenient methods. Adeno-associated viruses of any serotype are suitable (See, e.g., Blacklow, pp. 165-174 of “Parvoviruses and Human Disease” J. R. Pattison, ed. (1988); Rose, Comprehensive Virology 3:1, 1974; P. Tattersall “The Evolution of Parvovirus Taxonomy” In Parvoviruses (J R Kerr, S F Cotmore. ME Bloom, RMLinden, C RParrish, Eds.) p 5-14, Rudder Arnold, London, U K (2006); and D E Bowles, J E Rabinowitz, R J Samulski “The Genus Dependovirus” (J R Kerr, SF Cotmore. ME Bloom, R M Linden, C R Parrish, Eds.) p 15-23, Rudder Arnold, London, UK (2006), the disclosures of which are hereby incorporated by reference herein in their entireties). Methods for purifying for vectors may be found in, for example, U.S. Pat. Nos. 6,566,118, 6,989,264, and 6,995,006 and WO/1999/011764 titled “Methods for Generating High Titer Helper-free Preparation of Recombinant AAV Vectors”, the disclosures of which are herein incorporated by reference in their entirety. Preparation of hybrid vectors is described in, for example, PCT Application No. PCTIUS2005/027091, the disclosure of which is herein incorporated by reference in its entirety. The use of vectors derived from the AAVs for transferring genes in vitro and in vivo has been described (See e.g., International Patent Application Publication Nos: 91/18088 and WO 93/09239; U.S. Pat. Nos. 4,797,368, 6,596,535, and 5,139,941; and European Patent No: 0488528, all of which are herein incorporated by reference in their entirety). These publications describe various AAV-derived constructs in which the rep and/or cap genes are deleted and replaced by a gene of interest, and the use of these constructs for transferring the gene of interest in vitro (into cultured cells) or in vivo (directly into an organism). The replication defective recombinant AAVs according to the invention can be prepared by co-transfecting a plasmid containing the nucleic acid sequence of interest flanked by two AAV inverted terminal repeat (ITR) regions, and a plasmid carrying the AAV encapsidation genes (rep and cap genes), into a cell line that is infected with a human helper virus (for example an adenovirus). The AAV recombinants that are produced are then purified by standard techniques.

In some embodiments, the vector(s) for use in the methods of the invention are encapsidated into a virus particle (e.g. AAV virus particle including, but not limited to, AAV1, AAV2, AAV3, AAV4, AAV5, AAV6, AAV7, AAV8, AAV9, AAVIO, AAVII, AAV12, AAV13, AAV14, AAV15, and AAV16). Accordingly, the invention includes a recombinant virus particle (recombinant because it contains a recombinant polynucleotide) comprising any of the vectors described herein. Methods of producing such particles are known in the art and are described in U.S. Pat. No. 6,596,535.

A neuron-specific promoter allows precise manipulation of gene expression without affecting other cell types. Aspects of the present invention encompass expression cassettes and/or vectors comprising polynucleotide sequences of interest for expression in targeted cells. The polynucleotides can comprise promoters operably linked to gRNAs directed to HRH1 coding sequence. Targeted expression is accomplished using a cell-selective or cell-specific promoter. Examples are promoters for somatostatin, parvalbumin, GABAa6, L7, and calbindin. Other cell specific promoters can be promoters for kinases such as PKC, PKA, and CaMKII; promoters for other ligand receptors such as NMDAR1, NNIDAR2B, GluR2; promoters for ion channels including calcium channels, potassium channels, chloride channels, and sodium channels; and promoters for other markers that label classical mature and dividing cell types, such as calretinin, nestin, and beta3-tubulin.

Promoters of particular interest are RGC specific promoters, e.g. murine γ-synuclein (mSncg) promoter, which drives specific, potent and sustained transgene expression in rodent RGCs, nonhuman primate RGCs, and human primary RGCs, as well as human induced Pluripotent Stem Cell (IPS) stem cell-derived RGCs. See, for example,

In some embodiments a promoter is used for the selective expression of an operably linked gene in retinal ganglion cells (RGCs). In some embodiments the promoter comprises or consists of an mSncg promoter, optionally selected from the sequence set forth in SEQ ID NO:3, 4, 5, or 6, or a sequence having at least 95% sequence identity to a sequence on SEQ ID NO:3, 4, 5, or 6. In some embodiments the promoter sequence is provided in the context of a vector for expression, including without limitation a viral vector, e.g. an AAV vector. Cells of interest for expression include, without limitation, cells in the eye and progenitors thereof, e.g. retinal cells, particularly retinal ganglion cells, and their progenitors.

Compositions and methods for treating a mammalian subject for an optic nerve (ON) neuropathy and/or reducing or ameliorating degeneration of axons and/or soma of RGCs are provided. Aspects of the composition include a neuroprotective agent and a pharmaceutically acceptable excipient. The compositions may, alternatively, be in the form of a therapeutic gene therapy which include a mammalian viral vector, comprising a murine-synuclein promoter, or functional fragment thereof, that promotes expression of a transgene specifically in retinal ganglion cells (RGCs), said promoter in operable linkage with an expression cassette encoding the transgene, wherein the expressed transgene inhibits activity of an expression product of an endogenous HRH1 gene involved in an ER stress and/or UPR pathway that leads to axon or soma degeneration in the RGCs. Aspects of the methods include intravitreally or systemically administering the composition to treat the subject for the ON neuropathy. A variety of ON neuropathies may be treated by practicing the methods, including retinal ganglion cell degeneration, glaucoma, optic neuritis, ON traumatic injury and other ON-related diseases.

In some embodiments of the composition, the transgene encodes a CRISPER/Cas9 system. In some embodiments, the transgene encodes a protein that binds to the expression product of the endogenous HRH1 gene.

In some embodiments, the composition comprises an AAV vector, which comprises a murine γ-synuclein promoter in operable linkage with a nucleic acid encoding a Cas9 nuclease; and a U6 promoter in operable linkage with at least one guide (gRNA) 20-21 nucleotides in length, wherein each gRNA precedes an −NGG protospacer, and wherein each gRNA targets HRH1 for elimination by the CRISPER/Cas9 system. In some embodiments of the composition, the murine γ-synuclein promoter is selected from mSncg, mSncg-1.45 kb (SEQ ID NO: 3), mSncg-1.03 kb (SEQ ID NO: 4), mSncg-0.66 kb (SEQ ID NO: 5), mSncg-0.27 kb (SEQ ID NO: 6), mSncg shorter promoter (76CMVe+189mSncg) (SEQ ID NO: 7).

In some aspects, provided herein is a method of treating an optic nerve (ON) neuropathy in a mammalian subject in need thereof, comprising intravitreally or systemically administering the composition into the subject, thereby treating the ON neuropathy.

In some aspects, provided herein is a method of reducing or ameliorating degeneration of axons and/or soma of RGCs, comprising intravitreally or systemically administering the composition into a mammalian subject experiencing or at imminent risk of an ON neuropathy.

In some aspects, provided herein is a method of inducing neuroprotection/increasing survival/promoting functional recovery of RGC somata and axons, comprising intravitreally or systemically administering the composition into a mammalian subject experiencing or at imminent risk of an ON neuropathy. In some embodiments of the method, the ON neuropathy is retinal ganglion cell degeneration, including glaucoma, optic neuritis, ON traumatic injury and other ON-related diseases.

In some aspects, provided herein is a kit comprising a neuroprotective agent wherein the neuroprotective agents inhibits the activity or function of a HRH1 protein. In some aspects, provided herein is a kit comprising an AAV vector, wherein the vector comprises a murine γ-synuclein promoter that promotes expression of a transgene specifically in RGCs, wherein the murine γ-synuclein promoter is in operable linkage with an expression cassette that expresses Cas9 and a gRNA, wherein the sgRNA targets HRH1 gene in an ER stress and/or UPR pathway, wherein uninhibited activity of the endogenous HRH1 gene leads to axon or soma degeneration in the RGCs; and instructions for use.

### Conditions for Treatment

In another embodiment, the neuroprotective agent is intended for use as a neuroprotective medicament wherein the optic neuropathy is caused by a neuronal injury resulting from a disease. In some instances, the optic neuropathy and/or neurodegenerative disorder treated according to the methods described herein may be an optic neuropathy such as Leber's hereditary optic neuropathy (LHON), Anterior ischemic optic neuropathy (AION), optic disc drusen (ODD), dominant optic atrophy (DOA), ON damage associated with glaucoma, or other CNS neurodegenerative disorder leading to ON degeneration. In some instances of the methods disclosed herein, the disease or disorder may involve inflammation leading to degeneration of the ON.

In one embodiment, the neurodegenerative disorder is an ophthalmic disorder such as glaucoma. Glaucomas are a group of eye disorders characterized by progressive optic nerve damage in which an important part is a relative increase in intraocular pressure (IOP) that can lead to irreversible loss of vision. Glaucomas are categorized as open-angle glaucoma or angle-closure glaucoma. The “angle” refers to the angle formed by the junction of the iris and cornea at the periphery of the anterior chamber. The angle is where >98% of the aqueous humor exits the eye via either the trabecular meshwork and the Schlemm canal or the ciliary body face and choroidal vasculature. Glaucomas are further subdivided into primary (cause of outflow resistance or angle closure is unknown) and secondary (outflow resistance results from a known disorder), accounting for >20 adult types. Another group of glaucoma patients does not have IOP elevation, which in general is called normal tension glaucoma (NTG). NTG is also associated with progressive optic nerve degeneration and RGC death. Thus they are also subject to this gene therapy treatment.

Axons of retinal ganglion cells travel through the optic nerve carrying visual information from the eye to the brain. Damage to these axons causes ganglion cell death with resultant optic nerve atrophy and patchy vision loss. Elevated intraocular pressure (IOP; in unaffected eyes, the average range is 11 to 21 mm Hg) plays a role in axonal damage, either by direct nerve compression or diminution of blood flow. However, the relationship between externally measured pressure and nerve damage is complicated. Of people with IOP >21 mm Hg (ie, ocular hypertension), only about 1 to 2%/year (about 10% over 5 years) develop glaucoma. Additionally, about one third of patients with glaucoma do not have IOP >21 mm Hg (known as low-tension glaucoma or normal-tension glaucoma).

IOP is determined by the balance of aqueous secretion and drainage. Elevated IOP is caused by inhibited or obstructed outflow, not oversecretion; a combination of factors in the trabecular meshwork (eg, dysregulation of extracellular matrix, cytoskeletal abnormalities) appear to be involved. In open-angle glaucoma, IOP is elevated because outflow is inadequate despite an angle that appears unobstructed. In angle-closure glaucoma, IOP is elevated when a physical distortion of the peripheral iris mechanically blocks outflow.

Symptoms and signs of glaucoma vary with the type of glaucoma, but the defining characteristic is optic nerve damage as evidenced by an abnormal optic disk and certain types of visual field deficits. Glaucoma is diagnosed when characteristic findings of optic nerve damage are present and other causes have been excluded. Elevated IOP makes the diagnosis more likely, but elevated IOP can occur in the absence of glaucoma and is not essential for making the diagnosis.

### Methods

As summarized above, aspects of the instant disclosure include methods of treating a subject for an optic neuropathy. A variety of neurodegenerative disorders also may be treated by practice of the methods described herein, particularly glaucoma, e.g. open-angle glaucoma or angle-closure glaucoma. In some embodiments, provided herein is a method of treating an optic nerve (ON) neuropathy in a mammalian subject in need thereof, comprising intravitreally or systemically administering the composition into the subject, thereby treating the ON neuropathy. In some embodiments, provided herein is a method of reducing or ameliorating degeneration of axons and/or soma of RGCs, comprising intravitreally or systemically administering the composition into a mammalian subject experiencing or at imminent risk of an ON neuropathy.

In some aspects provided herein is a method of inducing neuroprotection/increasing survival/promoting functional recovery of RGC somata and axons, comprising intravitreally or systemically administering the composition into a mammalian subject experiencing or at risk of an ON neuropathy. In some embodiments of the method, the ON neuropathy is retinal ganglion cell degeneration, including glaucoma, optic neuritis, ON traumatic injury and other ON-related diseases.

In some embodiments, the composition comprises a neuroprotective agent and a pharmaceutically acceptable excipient. In some embodiments the neuroprotective agent comprises a tricyclic inhibitor of HRH1. In some embodiments the neuroprotective agent comprises an AAV vector, which comprises a murine γ-synuclein promoter in operable linkage with a nucleic acid encoding a CRISPR/Cas9 system and at least one guide RNA (gRNA) directed to a HRH1 gene.

As summarized above, methods of treating a subject for an ON neuropathy may include administering to a subject in need thereof an effective amount of a composition comprising a neuroprotective agent or an AAV virus comprising a therapeutic vector reduces expression of activity of a gene or gene product in the ER stress and/or unfolded protein response (UPR) pathway(s). Without being bound by theory, the instant disclosure includes the discovery that HRH1, is specifically involved in the ER stress response in retinal ganglion cells (RGCs) and therefore is useful for targeting a transgenic expression product to RGCs.

With regard to the present disclosure, small molecules targeting ER stress molecules or adeno-associated virus (AAV)-mediated gene therapies targeting ER stress/UPR pathways can be developed based on these findings to prevent neurodegeneration, which can be translated into novel therapeutic approaches to preserve important neuronal functions. An AAV vector employing a mSncg promoter was created to specifically knock down HRH1 for neuroprotection of RGC and optic nerve. The instant invention provides a novel therapeutic target for neuroprotectant development. The AAV vector containing the mSncg promoter and new gRNA for CRISPR/Cas9 gene editing in RGCs for neuroprotection. Small chemical modulators have been developed to target these ER stress molecules for neuroprotection. Novel AAV vectors have also been designed to genetically modulate these ER stress molecules.

Provided herein are methods of targeting expression products of a HRH1 gene in an ER stress and/or UPR pathway, wherein uninhibited activity of the HRH1 gene expression products leads to axon or soma degeneration in the RGCs. In some instances, methods of the present disclosure may include targeting a HRH1 gene that is associated with an ER stress and/or UPR pathway.

Various subjects may be treated in the methods of the present disclosure. In some instances, treated subjects may be mammals, including but not limited to e.g., rodents (e.g., rats, mice, etc.), non-human primates (e.g., macaques, marmosets, tamarins, spider monkeys, owl monkeys, vervet monkeys, squirrel monkeys, baboons, chimpanzees, etc.), humans, and the like. In some instances, a treated subject may be an animal model (e.g., a rodent model, a non-human primate model, etc.) of an optic neuropathy and/or neurodegenerative disorder. In some instances, a treated subject may be a human subject, including but not limited to e.g., a human subject having an optic neuropathy and/or neurodegenerative disorder, a human subject at increased risk of developing an optic neuropathy and/or neurodegenerative disorder, a human subject of advanced age (e.g., at least 60 years of age, at least 65 years of age, at least 70 years of age, at least 75 years of age, at least 80 years of age, at least 85 years of age, at least 90 years of age, etc.), or a combination thereof. Treated subjects may or may not be symptomatic, e.g., subject may or may not display or have previously displayed one or more symptoms of an optic neuropathy and/or neurodegenerative disorder, including but not limited to e.g., those optic neuropathies and/or neurodegenerative disorders described herein.

Methods of the present disclosure may include administering to a subject a neuroprotective agent that specifically targets RGCs and reduces RGC degeneration. Useful neuroprotective agents for reducing RGC degeneration include agents that modulate the activity or function of a HRH1 gene product or an ER stress or UPR pathway dependent thereon. Useful agents include antagonists, e.g., antagonists of a HRH1 protein or gene.

As antagonists, any useful inhibitor of the subject target gene and/or encoded product thereof may be employed in the subject methods. Non-limiting examples of useful inhibitors include but are not limited to e.g., non-peptide small molecule antagonists, peptide antagonists, interfering RNAs (e.g., siRNA, shRNA, etc.), antibodies (e.g., neutralizing antibodies, function blocking antibodies, etc.), aptamers, and the like. In some instances, inhibitors may target, e.g., specifically bind to, specifically hybridize to, etc., a target protein or a nucleic acid encoding a target protein including where the protein shares 100% sequence identity or less than 100% sequence identity, including e.g., at least 99%, at least 98%, at least 97% at least 96%, at least 95%, at least 90%, at least 85%, at least 80%, etc., sequence identity, with a protein or amino acid sequence of a protein described herein. In some embodiments, the antagonist is a small molecule. In some embodiments, the small molecule may include, without limitation, amoxapine, desipramine, desloratadine, trifluoperazine, clomipramine, amitriptyline, quetiapine, olanzapine, maprotiline, doxepin, loxapine, integrated stress response inhibitor (ISRIB), norquetiapine, etc.

In some embodiments, the target protein or gene encoding the target protein corresponds to a Histamine receptor H1 (HRH1). The human HRH1 gene corresponds to NCBI Gene ID: 3269. The amino acid sequence to the human HRH1 protein is: MSLPNSSCLLEDKMCEGNKTTMASPQLMPLVVVLSTICLVTVGLNLLVLYAVRSERKLHTVGNL YIVSLSVADLIVGAVVMPMNILYLLMSKWSLGRPLCLFWLSMDYVASTASIFSVFILCIDRYRSVQ QPLRYLKYRTKTRASATILGAWFLSFLWVIPILGWNHFMQQTSVRREDKCETDFYDVTWFKVMT AIINFYLPTLLMLWFYAKIYKAVRQHCQHRELINRSLPSFSEIKLRPENPKGDAKKPGKESPWEVL KRKPKDAGGGSVLKSPSQTPKEMKSPVVFSQEDDREVDKLYCFPLDIVHMQAAAEGSSRDYVA VNRSHGQLKTDEQGLNTHGASEISEDQMLGDSQSFSRTDSDTTTETAPGKGKLRSGSNTGLDY IKFTWKRLRSHSRQYVSGLHMNRERKAAKQLGFIMAAFILCWIPYFIFFMVIAFCKNCCNEHLHM FTIWLGYINSTLNPLIYPLCNENFKKTFKRILHIRS (SEQ ID NO: 29). The mouse HRH1 gene corresponds to NCBI Gene ID: 15465. The amino acid sequence to the mouse HRH1 protein is: MSLPNTSSASEDKMCEGNRTAMASPQLLPLVVVLSSISLVTVGLNLLVLYAVRSERKLHTVGNLY IVSLSVADLIVGAVVMPMNILYLIMTKWSLGRPLCLFWLSMDYVASTASIFSVFILCIDRYRSVQQP LRYLRYRTKTRASATILGAWFLSFLWVIPILGWHHFTPLAPELREDKCETDFYNVTWFKIMTAIINF YLPTLLMLWFYVKIYKAVRRHCQHRQLTNGSLPTFLEIKLRSEDAKEGAKKPGKESPWGVQKRP SRDPTGGLDQKSTSEDPKVTSPTVFSQEGERETVTRPCFRLDVMQTQPVPEGDARGSKANDQ TLSQPKMDEQSLSTCRRISETSEDQTLVDRQSFSRTTDSDTSIEPGLGKVKARSRSNSGLDYIKV TWKRLRSHSRQYVSGLHLNRERKAAKQLGCIMAAFILCWIPYFIFFMVIAFCNSCCSEPVHMFTI WLGYINSTLNPLIYPLCNENFKKTFKKILHIRS (SEQ ID NO: 30).

The compositions (e.g., those including one or more neuroprotective agents that inhibit HRH1 gene products or a therapeutic gene therapy directed to HRH1) of this disclosure can be supplied in the form of a pharmaceutical composition. Any suitable pharmaceutical composition may be employed, described in more detail below. As such, in some instances, methods of the present disclosure may include administering one or more agents in a composition comprising an excipient (e.g., an isotonic excipient) prepared under sufficiently sterile conditions for administration to a mammal, e.g., a human.

Administration of an agent to a subject, as described herein, may be performed employing various routes of administration. The route of administration may be selected according to a variety of factors including, but not necessarily limited to, the condition to be treated, the formulation and/or device used, the patient to be treated, and the like. Routes of administration useful in the disclosed methods include but are not limited to oral and parenteral routes, such as intravenous (iv), intraperitoneal (ip), rectal, topical, ophthalmic, nasal, and transdermal. Formulations for these dosage forms are described herein.

Where the agent is a polypeptide, polynucleotide, analog or mimetic thereof, it may be introduced into tissues or host cells by any number of routes, including viral infection, microinjection, or fusion of vesicles. Jet injection may also be used for intramuscular administration, as described by Furth et al., Anal Biochem. (1992) 205:365-368. The DNA may be coated onto gold microparticles, and delivered intradermally by a particle bombardment device, or “gene gun” as described in the literature (see, for example, Tang et al., Nature (1992) 356:152-154), where gold microprojectiles are coated with the DNA, then bombarded into skin cells. For nucleic acid therapeutic agents, a number of different delivery vehicles find use, including viral and non-viral vector systems, as are known in the art.

Those of skill in the art will readily appreciate that dose levels can vary as a function of the specific compound, the nature of the delivery vehicle, and the like. Preferred dosages for a given compound are readily determinable by those of skill in the art by a variety of means.

In those embodiments where an effective amount of an active agent is administered to the subject, the amount or dosage is effective when administered for a suitable period of time, such as one week or longer, including two weeks or longer, such as 3 weeks or longer, 4 weeks or longer, 8 weeks or longer, etc., so as to evidence a reduction in the disorder, e.g., a reduction in a symptom of the disorder or in a marker of disease pathology. For example, an effective dose is the dose that, when administered for a suitable period of time, such as at least about one week, and maybe about two weeks, or more, up to a period of about 3 weeks, 4 weeks, 8 weeks, or longer, will reduce a symptom of the disorder, for example, by about 10% or more, by about 20% or more, e.g., by 30% or more, by 40% or more, or by 50% or more, in some instances by 60% or more, by 70% or more, by 80% or more, or by 90% or more, for example, and will halt progression of the disorder in the subject. In some instances, an effective amount or dose of active agent will not only slow or halt the progression of the disease condition but will also induce the reversal of the condition, i.e., will cause an improvement in the neurological health of the subject. For example, in some instances, an effective amount is the amount that when administered for a suitable period of time, for example, at least about one week, and/or about two weeks, or more, up to a period of about 3 weeks, 4 weeks, 8 weeks, or longer will improve, stabilize, or at least reduce the progression of a disorder in subject, for example 1.5-fold, 2-fold, 3-fold, 4-fold, 5-fold, in some instances 6-fold, 7-fold, 8-fold, 9-fold, or 10-fold or more relative to the subject's condition prior to administration.

In some instances, in those embodiments where an effective amount of an active agent is administered to the subject, the amount or dosage is effective when administered for a suitable period of time to result in a reduction in RGC degeneration in the subject. Such a reduction may manifest in various ways, including but not limited to e.g., an increase in the number, size or length of RGCs, or a reduction in the amount of degeneration of RGCs, or their axons or soma, or the like. In some instances, methods of the present disclosure may result in at least a 5%, e.g., at least a 10%, at least a 15%, at least a 20%, at least a 25%, at least a 30%, at least a 35%, at least a 40%, at least a 45%, at least a 50%, at least a 55%, at least a 60%, at least a 65%, at least a 70% at least a 75%, at least a 80%, e.g., reduction in RGC degeneration. In some instances, methods of the present disclosure may result in at least a 5%, e.g., at least a 10%, at least a 15%, at least a 20%, at least a 25%, at least a 30%, at least a 35%, at least a 40%, at least a 45%, at least a 50%, at least a 55%, at least a 60%, at least a 65%, at least a 70% at least a 75%, at least a 80%, e.g., increase in RGC number, size or length of RGC axons or somata. Various methods of assessing the amount of RGC degeneration or increase in number, size or length of RGC axons or somata may be employed, including invasive and non-invasive techniques, such as electrophysiology measurement for RGC neuronal function, visual acuity, OCT imaging, fundus imaging, histology studies of RGC somata and axons morphology.

A “therapeutically effective amount”, a “therapeutically effective dose” or “therapeutic dose” is an amount sufficient to effect desired clinical results (i.e., achieve therapeutic efficacy, achieve a desired therapeutic response, etc.). A therapeutically effective dose can be administered in one or more administrations. For purposes of this disclosure, a therapeutically effective dose of an agent that inhibits expression of a target gene (e.g., HRH1) and/or compositions is an amount that is sufficient, when administered to the individual, to palliate, ameliorate, stabilize, reverse, prevent, slow or delay the progression of the disease state (e.g., an optic neuropathy) by, for example, inhibiting gene expression product formation, or otherwise preventing the symptoms or clinical progression of a neurodegenerative disorder present in the subject.

An effective amount of a composition comprising a neuroprotective agent will depend, at least, on the particular method of use, the subject being treated, the severity of the affliction, and the manner of administration of the therapeutic composition. A “therapeutically effective amount” of a composition is a quantity of a specified compound sufficient to achieve a desired effect in a subject (host) being treated.

Therapeutically effective doses of a composition comprising a neuroprotective agent or pharmaceutical composition can be determined by one of skill in the art, with a goal of achieving local (e.g., tissue) concentrations 7 that are at least as high as the IC50 of an applicable compound disclosed herein.

The specific dose level and frequency of dosage for any particular subject may be varied and will depend upon a variety of factors, including the activity of the composition comprising a neuroprotective agent, the metabolic stability and length of action of that composition, the age, body weight, general health, sex and diet of the subject, mode and time of administration, rate of excretion, drug combination, and severity of the condition of the host undergoing therapy.

Conversion of an animal dose to human equivalent doses (HED) may, in some instances, be performed using the conversion table and/or algorithm provided by the U.S. Department of Health and Human Services, Food and Drug Administration, Center for Drug Evaluation and Research (CDER) in, e.g., Guidance for Industry: Estimating the Maximum Safe Starting Dose in Initial Clinical Trials for Therapeutics in Adult Healthy Volunteers (2005) Food and Drug Administration, 5600 Fishers Lane, Rockville, MD 20857; (the disclosure of which is incorporated herein by reference).

### Conversion of Animal Doses to Human Equivalent Doses Based on Body Surface Area

| | | To Convert Animal Dose in | |
|---|---|---|---|
| | To Convert Animal Dose in | mg/kg to HED^{a} in mg/kg, Either: | |
| | mg/kg to Dose in mg/m², | Divide | Multiply |
| Species | Multiply by kₘ | Animal Dose By | Animal Dose By |
| Human | 37 | — | — |
| Child (20 kg)^{b} | 25 | — | — |
| Mouse | 3 | 12.3 | 0.08 |
| Hamster | 5 | 7.4 | 0.13 |
| Rat | 6 | 6.2 | 0.16 |
| Ferret | 7 | 5.3 | 0.19 |
| Guinea pig | 8 | 4.6 | 0.22 |
| Rabbit | 12 | 3.1 | 0.32 |
| Dog | 20 | 1.8 | 0.54 |
| Primates: | | | |
| Monkeys^{c} | 12 | 3.1 | 0.32 |
| Marmoset | 6 | 6.2 | 0.16 |
| Squirrel monkey | 7 | 5.3 | 0.19 |
| Baboon | 20 | 1.8 | 0.54 |
| Micro-pig | 27 | 1.4 | 0.73 |
| Mini-pig | 35 | 1.1 | 0.95 |

^{a}Assumes 60 kg human. For species not listed or for weights outside the standard ranges, HED can be calculated from the following formula:
HED = animal dose in mg/kg × (animal weight in kg/human weight in kg)0.33.
^{b}This km value is provided for reference only since healthy children will rarely be volunteers for phase 1 trials.
^{c}For example, cynomolgus, rhesus, and stumptail.

### Pharmaceutical Compositions

A pharmaceutical composition comprising a neuroprotective agent (i.e., an antagonist or an AAV virus comprising a therapeutic vector) may be administered to a patient alone, or in combination with other supplementary active agents. The pharmaceutical compositions may be manufactured using any of a variety of processes, including, without limitation, conventional mixing, dissolving, granulating, dragee-making, levigating, emulsifying, encapsulating, entrapping, and lyophilizing. The pharmaceutical composition can take any of a variety of forms including, without limitation, a sterile solution, suspension, emulsion, lyophilisate, tablet, pill, pellet, capsule, powder, syrup, elixir or any other dosage form suitable for administration.

A composition comprising a neuroprotective agent may be administered to the host using any convenient means capable of resulting in the desired reduction in disease condition or symptom. Thus, a neuroprotective agent can be incorporated into a variety of formulations for therapeutic administration. More particularly, a neuroprotective agent can be formulated into pharmaceutical compositions by combination with appropriate pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

Formulations for pharmaceutical compositions are well known in the art. For example, Remington's Pharmaceutical Sciences, by E. W. Martin, Mack Publishing Co., Easton, Pa., 19th Edition, 1995, describes exemplary formulations (and components thereof) suitable for pharmaceutical delivery of disclosed compositions. Pharmaceutical compositions comprising at least one of the neuroprotective agents can be formulated for use in human or veterinary medicine. Particular formulations of a disclosed pharmaceutical composition may depend, for example, on the mode of administration and/or on the location of the infection to be treated. In some embodiments, formulations include a pharmaceutically acceptable carrier in addition to at least one active ingredient, such as a composition comprising a neuroprotective agent. In other embodiments, other medicinal or pharmaceutical agents, for example, with similar, related or complementary effects on the affliction being treated can also be included as active ingredients in a pharmaceutical composition.

Pharmaceutically acceptable carriers useful for the disclosed methods and compositions are conventional in the art. The nature of a pharmaceutical carrier will depend on the particular mode of administration being employed. For example, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can optionally contain minor amounts of non-toxic auxiliary substances (e.g., excipients), such as wetting or emulsifying agents, preservatives, and pH buffering agents and the like; for example, sodium acetate or sorbitan monolaurate. Other non-limiting excipients include, nonionic solubilizers, such as cremophor, or proteins, such as human serum albumin or plasma preparations.

Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

The disclosed pharmaceutical compositions may be formulated as a pharmaceutically acceptable salt of a disclosed neuroprotective agent. Pharmaceutically acceptable salts are non-toxic salts of a free base form of a compound that possesses the desired pharmacological activity of the free base. These salts may be derived from inorganic or organic acids. Non-limiting examples of suitable inorganic acids are hydrochloric acid, nitric acid, hydrobromic acid, sulfuric acid, hydroiodic acid, and phosphoric acid. Non-limiting examples of suitable organic acids are acetic acid, propionic acid, glycolic acid, lactic acid, pyruvic acid, malonic acid, succinic acid, malic acid, maleic acid, fumaric acid, tartaric acid, citric acid, benzoic acid, cinnamic acid, mandelic acid, methanesulfonic acid, ethanesulfonic acid, p-toluenesulfonic acid, methyl sulfonic acid, salicylic acid, formic acid, trichloroacetic acid, trifluoroacetic acid, gluconic acid, asparagic acid, aspartic acid, benzenesulfonic acid, p-toluenesulfonic acid, naphthalenesulfonic acid, and the like. Lists of other suitable pharmaceutically acceptable salts are found in Remington's Pharmaceutical Sciences, 17th Edition, Mack Publishing Company, Easton, Pa., 1985. A pharmaceutically acceptable salt may also serve to adjust the osmotic pressure of the composition.

A composition comprising a neuroprotective agent can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents. Such preparations can be used for oral administration.

A composition comprising a neuroprotective agent can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives. The preparation may also be emulsified or the active ingredient encapsulated in liposome vehicles. Formulations suitable for injection can be administered by an intravitreal, intraocular, intramuscular, subcutaneous, sublingual, or other route of administration, e.g., injection into the gum tissue or other oral tissue. Such formulations are also suitable for topical administration.

In some embodiments, a composition comprising a neuroprotective agent can be delivered by a continuous delivery system. The term “continuous delivery system” is used interchangeably herein with “controlled delivery system” and encompasses continuous (e.g., controlled) delivery devices (e.g., pumps) in combination with catheters, injection devices, and the like, a wide variety of which are known in the art.

Furthermore, a composition comprising a neuroprotective agent can be made into suppositories by mixing with a variety of bases such as emulsifying bases or water-soluble bases. A composition comprising a neuroprotective agent or therapeutic vector can be administered rectally via a suppository. The suppository can include vehicles such as cocoa butter, carbowaxes and polyethylene glycols, which melt at body temperature, yet are solidified at room temperature.

The term “unit dosage form,” as used herein, refers to physically discrete units suitable as unitary dosages for human and animal subjects, each unit containing a predetermined quantity of a composition comprising a neuroprotective agent calculated in an amount sufficient to produce the desired effect in association with a pharmaceutically acceptable diluent, carrier or vehicle. The specifications for a composition comprising a neuroprotective agent depend on the particular neuroprotective agent employed and the effect to be achieved, and the pharmacodynamics associated with each compound in the host.

The dosage form of a disclosed pharmaceutical composition will be determined by the mode of administration chosen. For example, in addition to injectable fluids, topical or oral dosage forms may be employed. Topical preparations may include eye drops, ointments, sprays and the like. In some instances, a topical preparation of a medicament useful in the methods described herein may include, e.g., an ointment preparation that includes one or more excipients including, e.g., mineral oil, paraffin, propylene carbonate, white petrolatum, white wax and the like, in addition to one or more additional active agents.

Oral formulations may be liquid (e.g., syrups, solutions or suspensions), or solid (e.g., powders, pills, tablets, or capsules). Methods of preparing such dosage forms are known, or will be apparent, to those skilled in the art.

Certain embodiments of the pharmaceutical composition comprising a neuroprotective agent may be formulated in unit dosage form suitable for individual administration of precise dosages. The amount of active ingredient administered will depend on the subject being treated, the severity of the affliction, and the manner of administration, and is known to those skilled in the art. Within these bounds, the formulation to be administered will contain a quantity of the extracts or compounds disclosed herein in an amount effective to achieve the desired effect in the subject being treated.

Each therapeutic composition can independently be in any dosage form, such as those described herein, and can also be administered in various ways, as described herein. For example, the compositions may be formulated together, in a single dosage unit (that is, combined together in one form such as capsule, tablet, powder, or liquid, etc.) as a combination product. Alternatively, when not formulated together in a single dosage unit, an individual composition comprising a neuroprotective agent may be administered at the same time as another therapeutic composition or sequentially, in any order thereof.

In some instances, methods of treating a subject as described herein may include administering to the subject an effective amount of an agent that reduces RGC degeneration in the subject, as identified in a method of screening described herein.

### Reagents, Devices and Kits

Also provided are reagents, devices and kits thereof for practicing one or more of the above-described methods. The subject reagents, devices and kits thereof may vary greatly. Reagents and devices of interest include those mentioned above with respect to the methods of treating a neurodegenerative condition in a subject, including by administering to the subject an effective amount of an agent that reduces the prevalence of RGC degeneration. The subject kits may include any combination of components (e.g., reagents, cell lines, etc.) for performing the subject methods, such as e.g., methods of treating a neurodegenerative condition and/or methods of identifying a RGC degeneration-associated target gene.

In some embodiments, a subject kit may be employed in a method of identifying a target gene associated with degeneration of retinal ganglion cells. Such kits may vary and may, but need not necessarily, include one or more RGC populations. In some embodiments, a subject kit may include one or more, including a plurality of or a library of, CRISPR-based gene silencing agents. In some embodiments, the subject kits may include a nucleic acid for expressing a Cas9 polypeptide within a particular cell type, such as a retinal ganglion cell. In some instances, a cell line contained within a subject kit may be configured (e.g., genetically modified) to express a Cas9 polypeptide.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, portable flash drive, etc., on which the information has been recorded. Yet another means that may be present is a website address which may be used via the internet to access the information at a removed site. Any convenient means may be present in the kits.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

### Example 1

When the protein or calcium homeostasis of the endoplasmic reticulum (ER) is adversely altered, cells experience ER stress that leads to various diseases including neurodegeneration. Genetic deletion of an ER stress downstream effector, CHOP, significantly protects neuron somata and axons. Here we report that three tricyclic compounds identified through a small-scale high throughput screening using a CHOP promoter-driven luciferase cell-based assay, effectively inhibit ER stress by antagonizing their common target, histamine receptor H1 (HRH1). We further demonstrated that systemic administration of one of these compounds, maprotiline, or CRISPR-mediated retinal ganglion cell (RGC)-specific HRH1 inhibition, delivers considerable neuroprotection of both RGC somata and axons and preservation of visual function in two mouse optic neuropathy models. Finally, we determine that maprotiline restores ER homeostasis by inhibiting HRH1-mediated Ca^{2 +} release from ER. In this work we establish maprotiline as a candidate neuroprotectant and HRH1 as a potential therapeutic target for glaucoma.

We previously found that ON injury induces neuronal endoplasmic reticulum (ER) stress in RGCs, suggesting a detrimental role of RGC-specific ER stress in glaucoma. When the protein or calcium homeostasis of the ER is adversely altered, cells experience ER stress and activate three signaling pathways initiated by three ER-resident stress-sensing proteins: inositol-requiring protein-1 (IRE1α), activating transcription factor-6 (ATF6) and protein kinase RNA-like ER kinase (PERK), together called the unfolded protein response (UPR). IRE1α, a bi-functional enzyme that contains both a Ser/Thr kinase domain and an endoribonuclease (RNase) domain, mediates the splicing of X-box binding protein 1 (XBP-1) mRNA to generate an active (spliced) form of the transcription factor, XBP-1s. The IRE1α-XBP-1s pathway targets genes that increase ER protein-folding capacity and facilitate degradation of misfolded proteins. On the other hand, IRE1α kinase activity also activates pro-apoptotic c-Jun kinase (JNK), which contributes to Bax-dependent IRE1α-induced apoptosis. ATF6 is a transcription factor that is truncated and thereby activated by ER stress to control the expression of a group of UPR target genes. PERK phosphorylates and inactivates eukaryotic translation initiation factor 2α (eIF2α) to attenuate global cap-dependent mRNA translation and thereby reduce protein load on the ER. Activating transcription factor 4 (ATF4) downstream of PERK-eIF2α induces expression of ER stress-specific transcription factor C/EBP homologous protein (CHOP). CHOP is a well-known pro-apoptotic transcription factor that mediates ER stress-induced cell death by downregulating anti-apoptotic Bcl2, upregulating pro-apoptotic BH-3 only molecules Bim and PUMA, increasing expression of death receptor 5 (DR5) and caspase 8 cleavage. ATF4 can also form heterodimers with CHOP to cause cell death by upregulating protein synthesis and inducing oxidative stress. Chronic ER stress with prolonged PERK-eIF2α-ATF4-CHOP signaling has been associated with many acute and chronic neurodegenerative diseases; genetic manipulation and small molecular modulators of this pathway have proven to be beneficial in animal models of various neurodegenerative diseases.

We also previously found that genetic inhibition of CHOP or its upstream regulator eIF2α significantly protects RGCs' somata and axons and preserves visual functions in mouse models of traumatic ON injury, glaucoma, and optic neuritis. Therefore, identification of small modulators of ER stress to block CHOP is an important step toward developing effective neuroprotectants for glaucoma. We reasoned that identifying CHOP inhibitors from FDA-approved drugs would significantly shorten the drug development process. Therefore, we used a reporter cell line expressing CHOP promoter-driven luciferase to perform a high throughput screening (HTS) of five compound libraries with a total of 4846 compounds that have known bioactivities and have been approved for clinical application.

In this work, we identify three FDA approved drugs, amoxapine, desloratadine and maprotiline, as potent ER stress inhibitors. They share similar tricyclic chemical structures and a common antagonistic target, histamine receptor H1 (HRH1), through which we find that they restore ER homeostasis and achieve significant neuroprotection of RGCs and ONs in vivo in two mouse optic neuropathy models. Finally, we determine that maprotiline inhibits HRH1-mediated ER Ca^{2 +} release and thereby inhibits the damaging intracellular Ca^{2 +} influx induced by axon injury. This readily testable small molecule drug is a promising neuroprotectant, and HRH1 is a potential therapeutic target for glaucoma and other neurodegenerative diseases associated with ER stress.

### Results

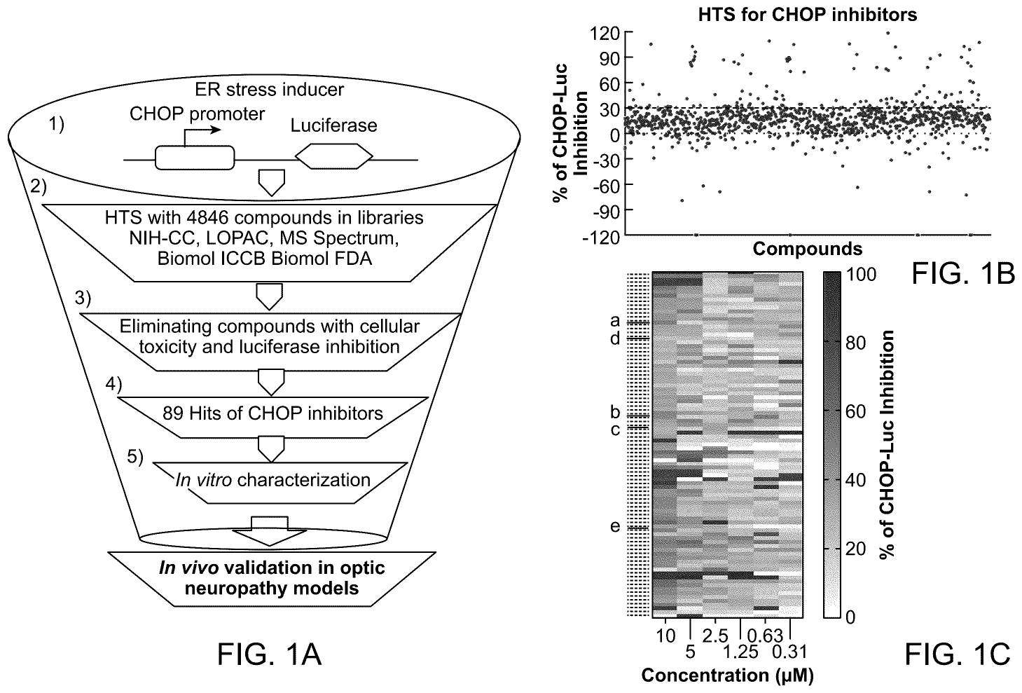

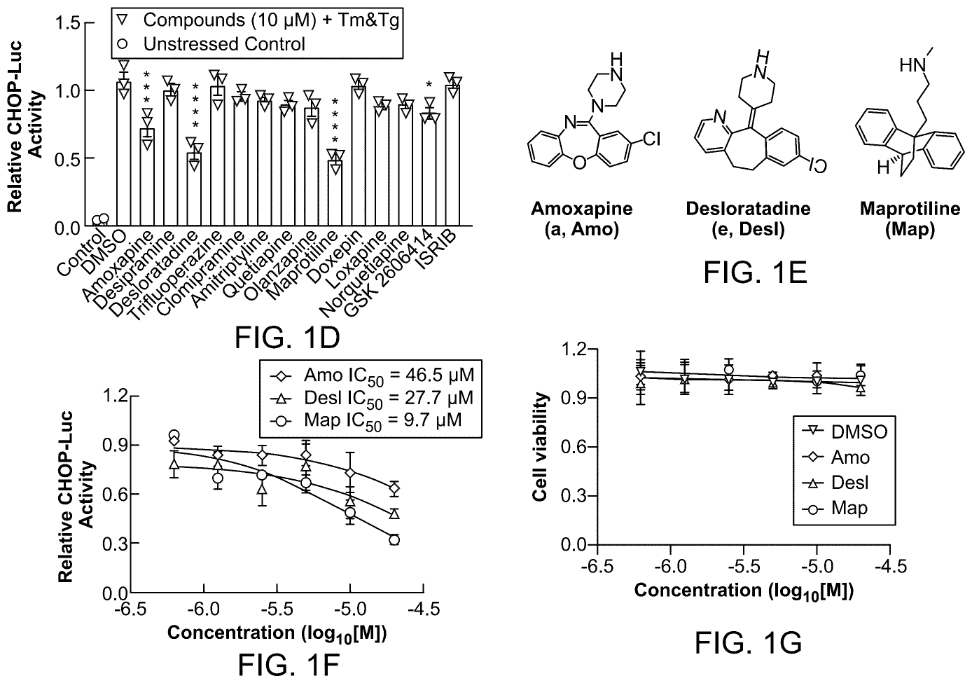

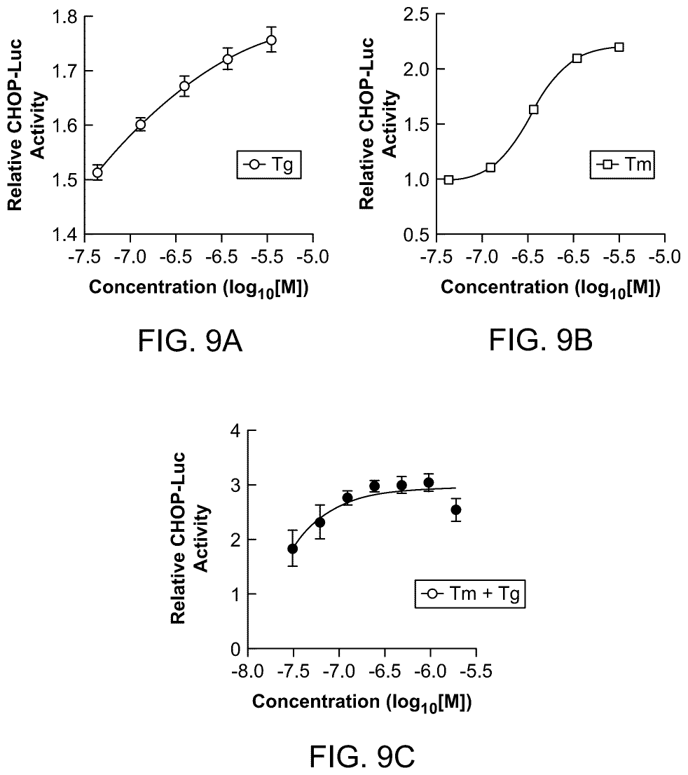

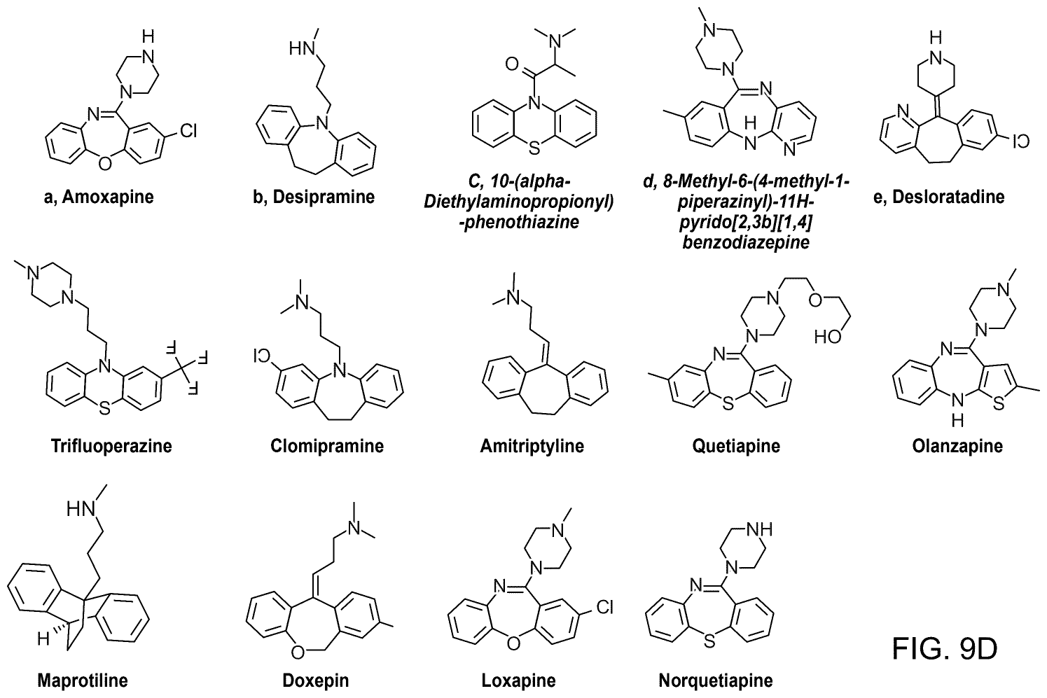

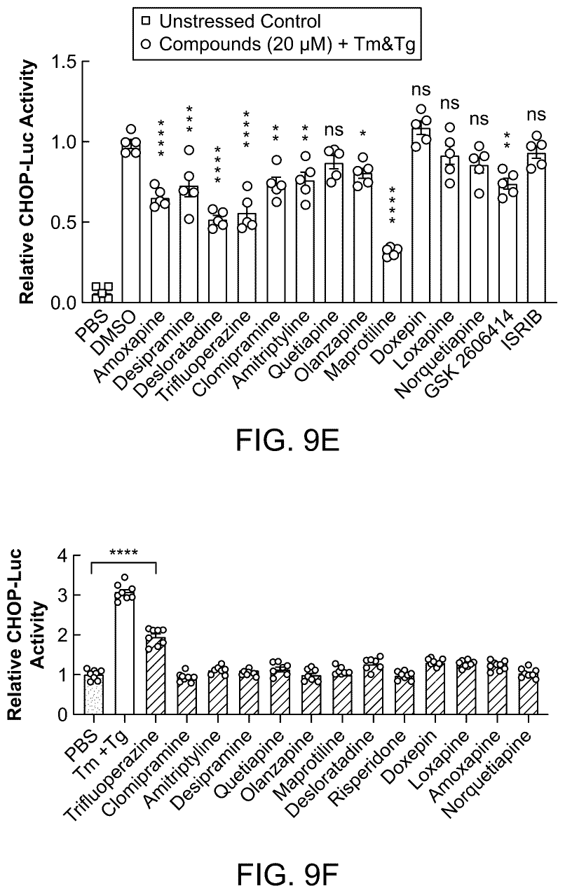

CHOP pathway inhibitors identified through cell-based HTS of FDA-approved drug libraries. We generated a stable cell line to express CHOP promoter-driven luciferase (CHOP-Luc) in HEK293T cells. This promoter shows dose-dependent responses to the ER stress inducers thapsigargin (Tg) and tunicamycin (Tm) (FIG. 9a-c). We used this reporter cell line in a multiple dose-response assay to screen 4846 compounds from five FDA-approved drug libraries. All compounds were run in 7-point dose response based on a previous publication, except the NIH Clinical Collection (446 compounds), which were tested in duplicate due to the small quantity of compounds on hand. We identified 89 “hits” (FIG. 1a-c) based on the criteria: 1)>30% inhibition of Tm/Tg induced CHOP-Luc signal; 2) no cell toxicity or luciferase inhibition activity; 3) dose-dependent effect. Among the 89 hit compounds, five (a-e) with good dose-dependent inhibition of CHOP-Luc share a similar tricyclic chemical structure (FIG. 9d). We then focused on this series of compounds and added nine more tricyclic or tetracyclic FDA-approved small molecule drugs (not in the libraries) for retesting with the CHOP-Luc reporter line; we excluded compounds “c” and “d” because they are not commercially available (FIG. 9d). Among the 12 retested compounds, only three (amoxapine, desloratadine and maprotiline) significantly inhibited CHOP-luciferase activity induced by ER stress (Tm/Tg) at 10 UM (FIG. 1d). Two known inhibitors of the PERK-CHOP pathway, GSK2606414 and ISRIB, were used as reference compounds. However, only GSK2606414 but not ISRIB significantly inhibited CHOP-luciferase activity induced by Tm/Tg (FIG. 1d and FIG. 9e), indicating the low sensitivity of the reporter line. Another five compounds (desipramine, trifluoperazine, clomipramine, amitriptyline, olanzapine) inhibited CHOP-luciferase activity at 20 μM (FIG. 9e). Interestingly, a previous study also identified trifluoperazine as a CHOP inhibitor using a different CHO luciferase reporter cell line driven by murine CHOP promoter. Maprotiline appeared to be the most potent of the three as it has the lowest IC₅₀ (FIG. 1e,f). Importantly, these three drugs had no obvious cell toxicity within the concentration ranges that we tested (FIG. 1g) and they did not induce ER stress by themselves (FIG. 9f).

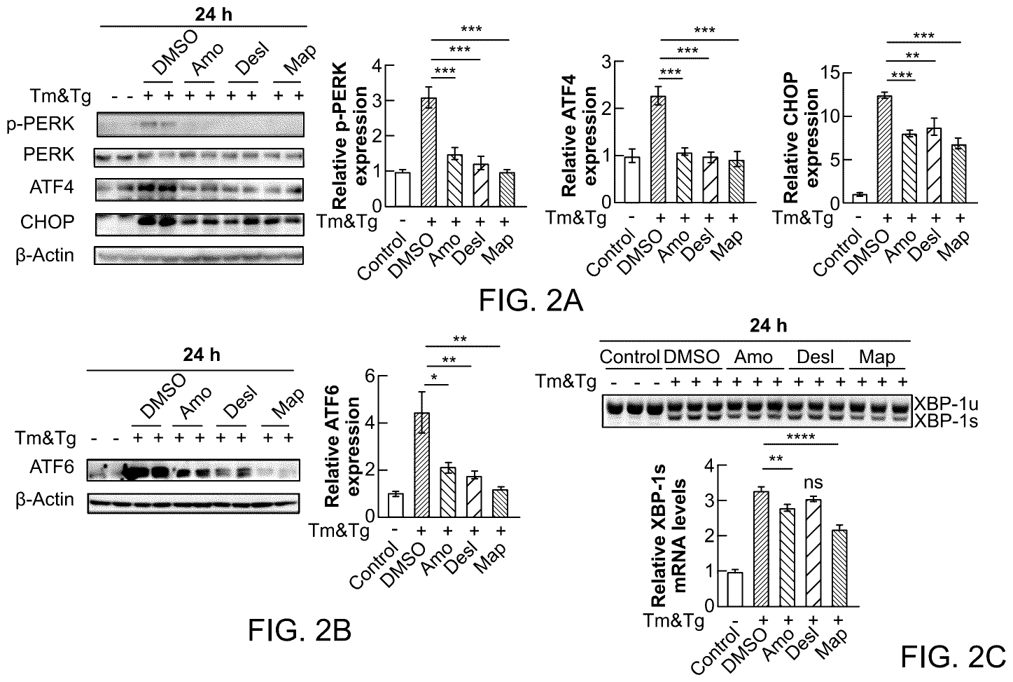

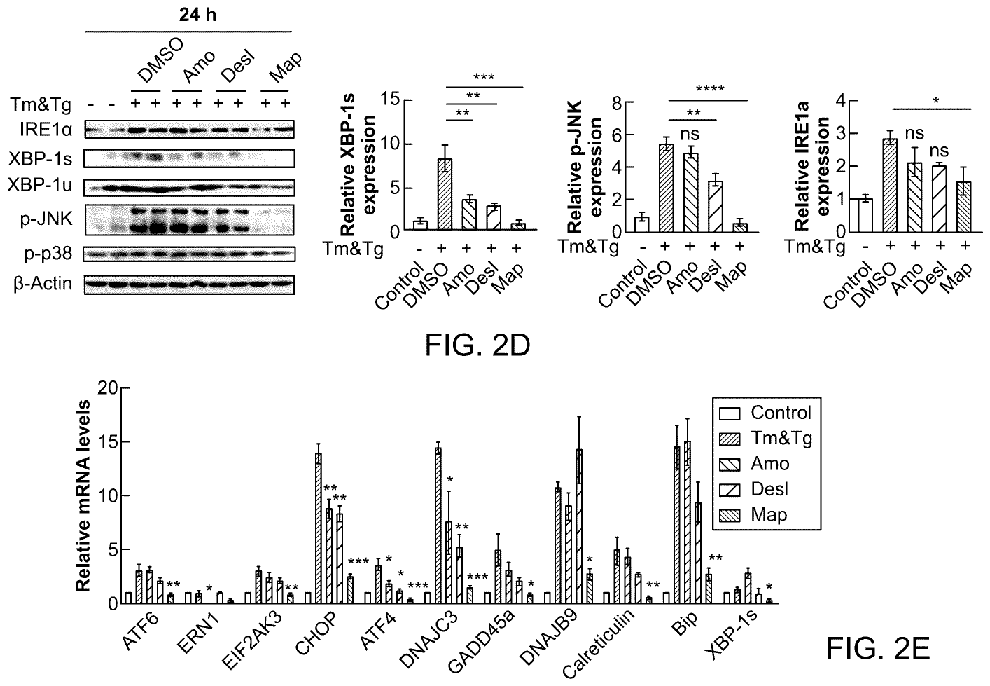

Amoxapine, desloratadine and maprotiline inhibit all three UPR pathways induced by ER stress. To determine whether these compounds inhibit the CHOP branch of UPR preferentially or act as general ER stress modulators that also affect the other two branches, we examined the signaling cascades downstream of the three UPR pathways induced by ER stress after treatment of HEK293T cells with each of these compounds. We again used Tm/Tg (1 μM) to induce ER stress and compared the three hit compounds (10 μM) to DMSO control: 1) The three compounds significantly inhibited PERK phosphorylation and expression of ATF4 and CHOP, indicating upstream modulation of the PERK-CHOP pathway (FIG. 2a). 2) The three compounds also significantly inhibited ATF6 expression (FIG. 2b). 3) Amoxapine and maprotiline likewise significantly inhibited the third UPR pathway downstream of IRE1α activation, as indicated by XBP-1 mRNA splicing (FIG. 2c). All three compounds also downregulated XBP-1s protein level (FIG. 2d). JNK phosphorylation is another downstream effector of IRE1α and contributes to cell death, especially in glaucoma. Desloratadine and maprotiline significantly inhibited JNK phosphorylation and maprotiline inhibited IRE1α phosphorylation (FIG. 2d). Lastly, we used qPCR as additional confirmation that these three compounds inhibited various downstream genes of UPR at the mRNA levels; maprotiline showed the most striking modulation of all the UPR pathways (FIG. 2e).

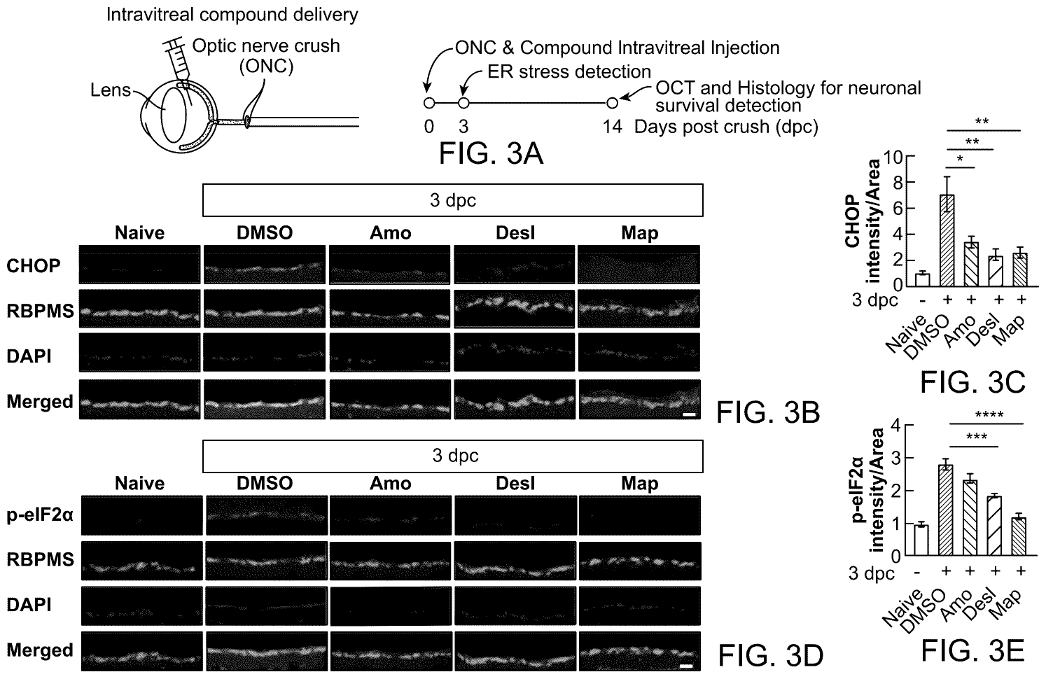

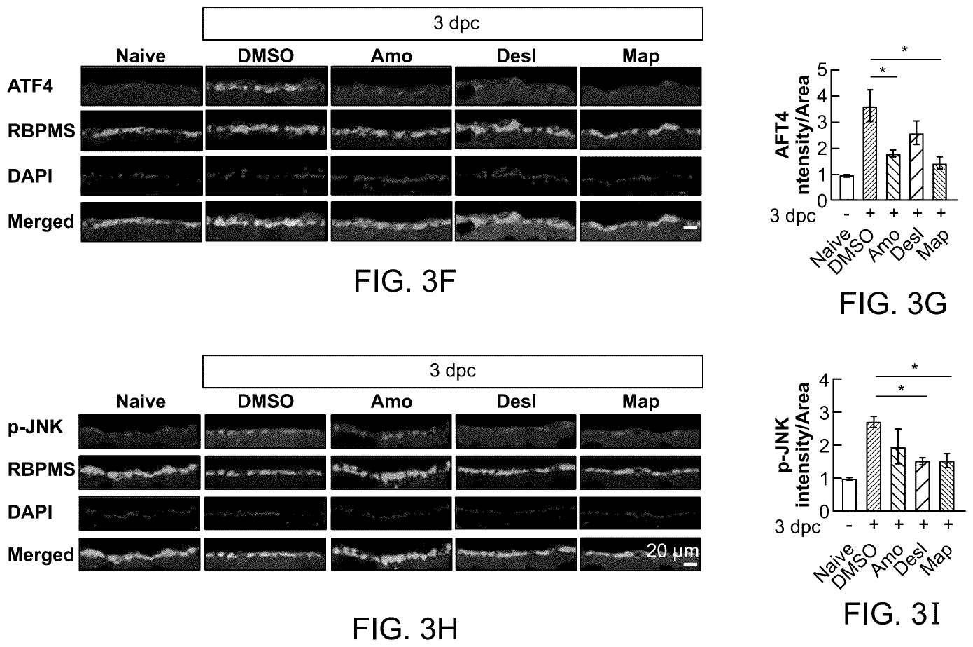

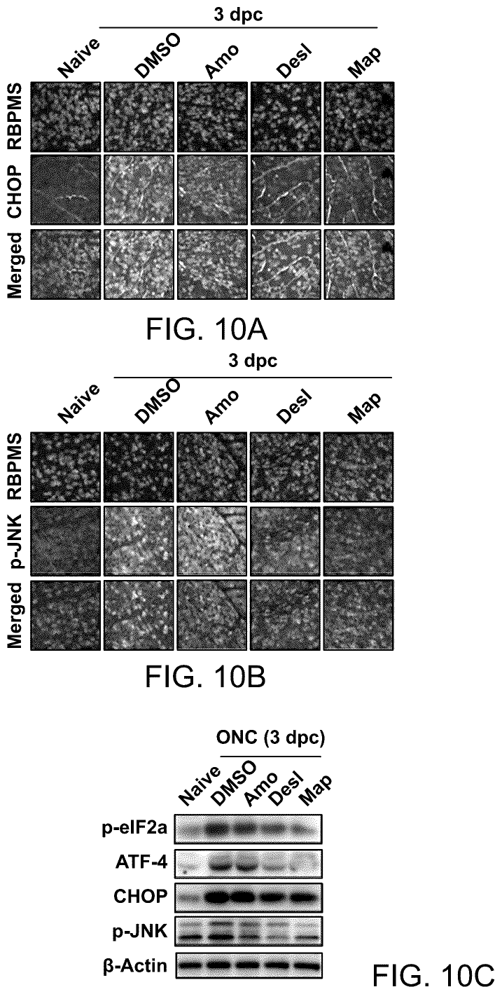

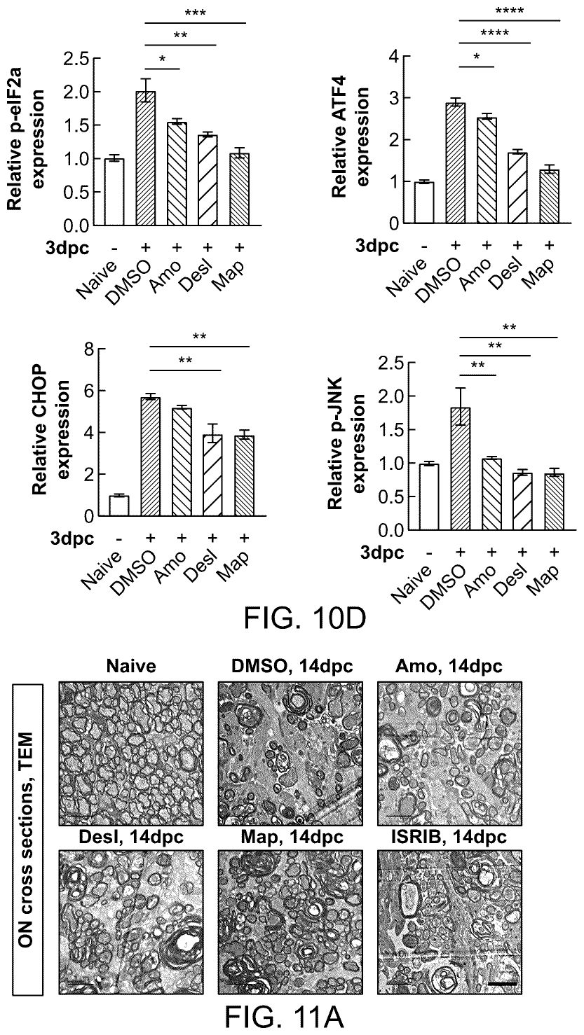

The three compounds inhibit neuronal ER stress and provide significant neuroprotection in the in vivo mouse traumatic ON crush (ONC) model. We previously demonstrated that traumatic ON injury induces ER stress in RGCs at 3 days post crush (3dpc). To determine the in vivo effects of these three ER stress modulators, we delivered each of the three compounds into mouse eyes by intravitreal injection on the same day after ON crush (ONC) and examined the expression of the key ER stress molecules in RGCs at 3dpc and the survival of RGC somata and axons at 14dpc (FIG. 3a). Consistent with our cell-based in vitro assays, local administration of the three compounds significantly inhibited ONC-induced CHOP and ATF4 expression, and the phosphorylation of eIF2α and JNK in RGCs examined in both retinal sections (FIG. 3b-i) and retinal wholemounts (FIG. 10a,b). Interestingly, western Blot assays of ON lysates demonstrated that the three compounds also inhibited ER stress molecules elevated by ONC in the ONs (FIG. 10c,d).

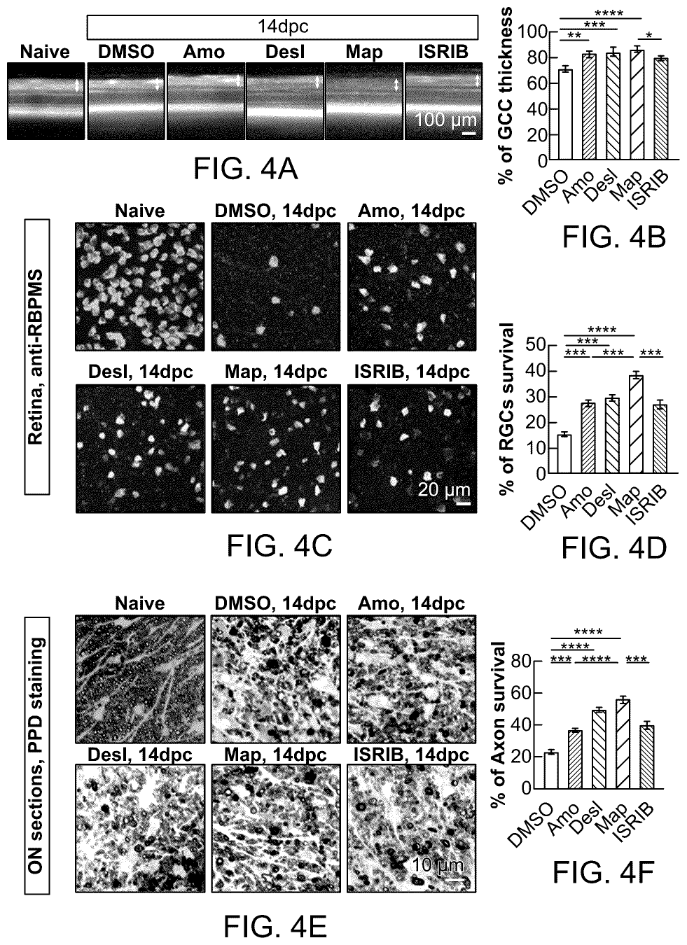

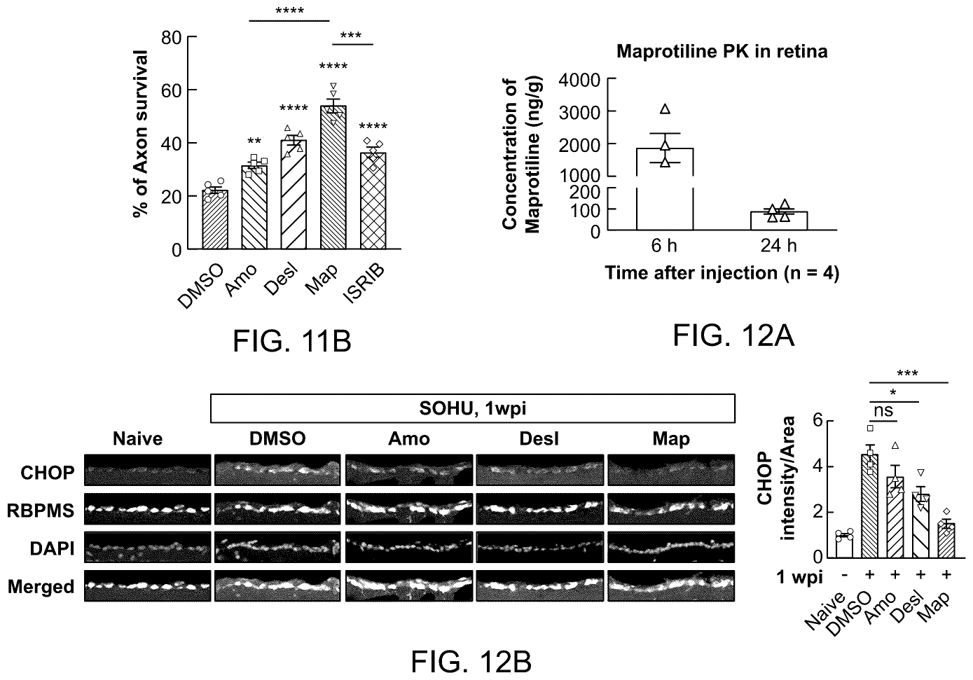

ONC is extensively used as a traumatic optic neuropathy model that injures all RGC axons and causes universal RGC and ON degeneration. Therefore, we next used this model to examine the effects of the three ER stress inhibitors on RGC soma and axon survival. We again delivered the three compounds into one of the mouse eyes by intravitreal injection on the same day as ONC, left the contralateral eye as internal control, and then maintained the compound exposure by daily intraperitoneal (i.p.) injection to avoid repeated intravitreal injection. The control group was treated with vehicle (DMSO). We have used optical coherence tomography (OCT) before to image and measure retina thickness in living animals as an accurate in vivo morphological readout for RGC degeneration. OCT images at 14dpc showed significant thinning of the ganglion cell complex (GCC), in crushed eyes treated with DMSO compared to contralateral naïve eyes, whereas crushed eyes treated with amoxapine, desloratadine, or maprotiline showed significantly thicker GCC than DMSO treated eyes (FIG. 4a,b), suggesting significant RGC neuroprotection by these compounds. Histological analysis of post-mortem retina wholemounts and ON semi-thin sections consistently demonstrated significant loss of RGC somata and axons at 14dpc in the DMSO control group, whereas the survival of RGCs and axons was much higher in the compound-treated eyes (FIG. 4c-f). We confirmed the axon protection effect of these compounds by TEM analysis of ON cross sections (FIG. 11). ISRIB is a small molecule inhibitor of the PERK pathway identified through cell-based screening. It showed no effect with the reporter cell line (FIG. 1d), but ISRIB provided neuroprotection in the ONC mouse model, to a lesser degree than maprotiline (FIG. 4a-f). Taken together, these results show that in vivo application of each of the three compounds (amoxapine, desloratadine, and maprotiline) effectively inhibits RGC ER stress and significantly protects RGCs and ONs after traumatic ON injury. Because maprotiline shows the most potent and consistent effects on ER stress modulation and neuroprotection both in vitro and in vivo, we focused on characterization of maprotiline in the subsequent experiments.

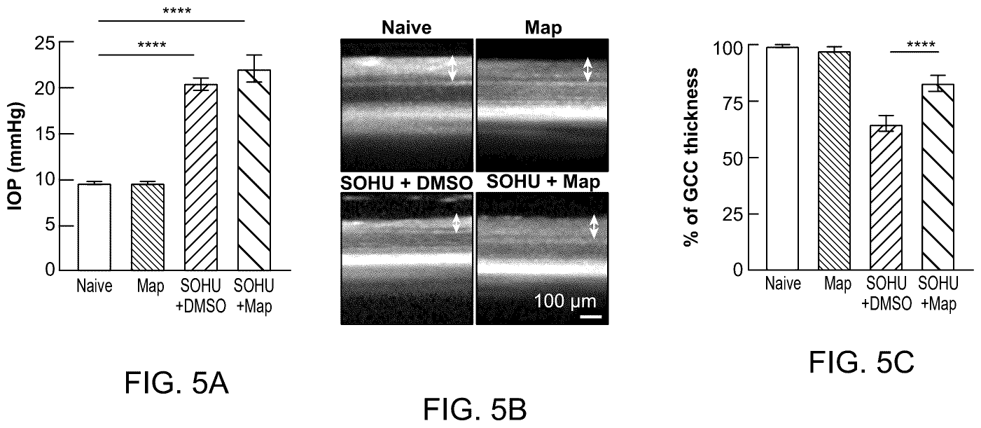

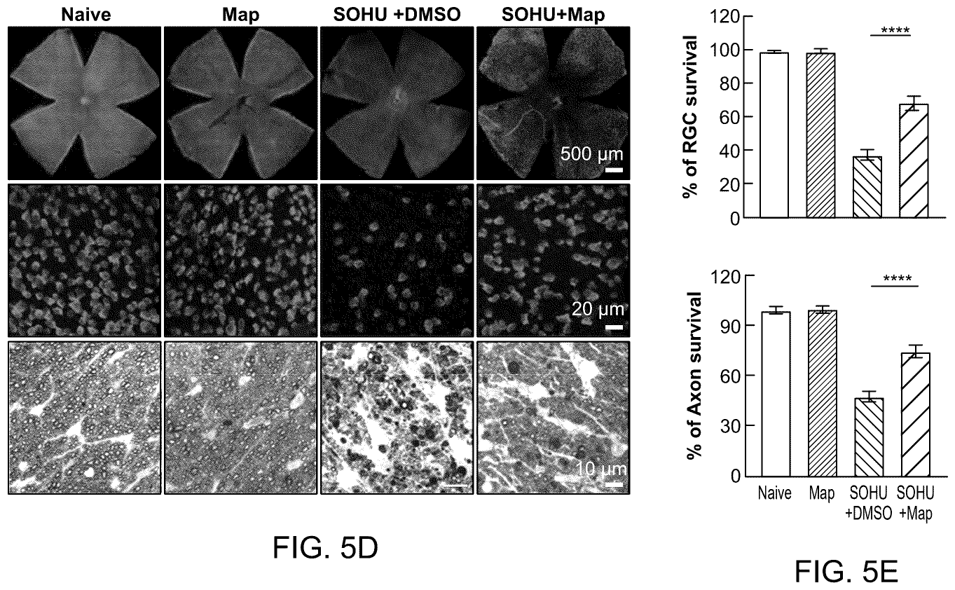

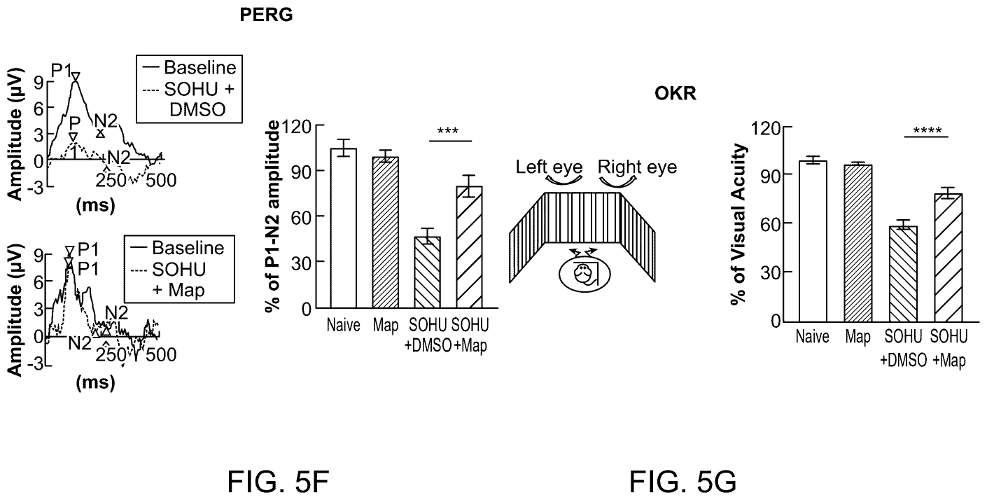

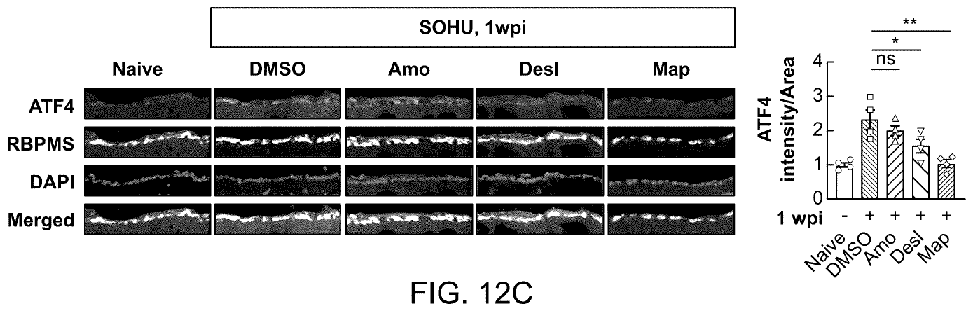

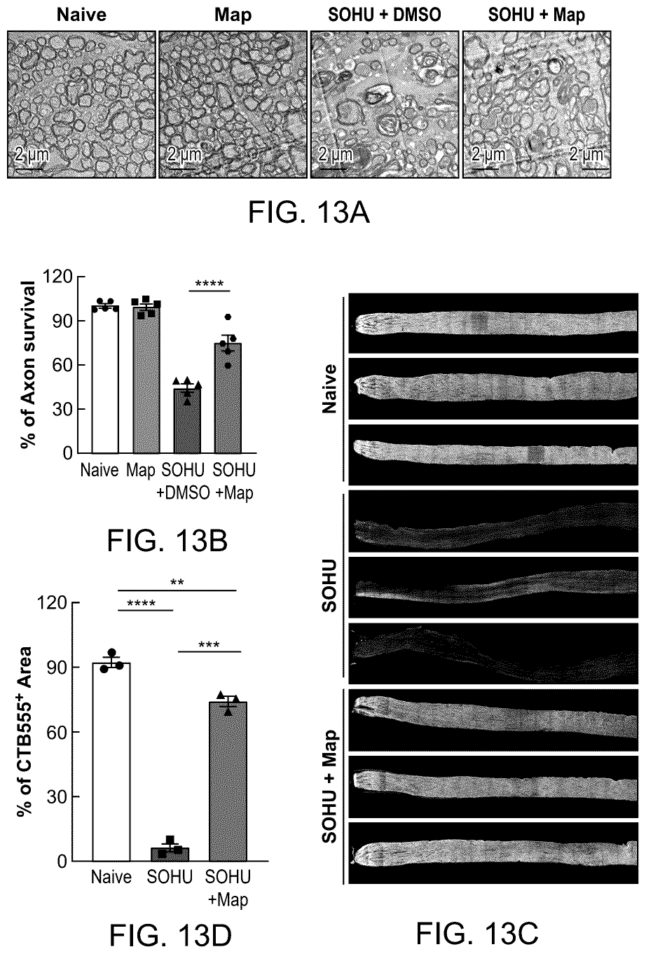

Maprotiline significantly promotes both RGC soma and axon survival and preserves visual functions in mouse SOHU glaucoma model. We previously developed the silicone oil-induced ocular hypertension (SOHU) mouse glaucoma model, which faithfully replicates human secondary glaucoma with persistent elevation of IOP and severe degeneration of RGCs and ON. To test the effect of maprotiline on glaucomatous neurodegeneration, we generated the SOHU glaucoma model in one eye, used the contralateral eye as sham control, and treated the animals both systemically by i.p. injection+by local retrobulbar injection of compounds or vehicle (DMSO). We performed i.p. injection daily based on the presence of maprotiline in the retina 6 and 24 hours after injection (FIG. 12a). We did not use intravitreal injection to deliver the drug directly into the eye because intravitreal injection itself can lower IOP and therefore compromise the ocular hypertension glaucoma model. CHOP and ATF4 expression were elevated in glaucomatous RGCs at one week post SO injection (1wpi) in the SOHU model; their expression was significantly inhibited by systematic administration of the three compounds. Next, we focused on maprotiline. Maprotiline treatment did not affect normal IOP in naïve mice, nor elevated IOP in SOHU mice (FIG. 5a). In vivo OCT retinal imaging showed significant thinning of the GCC at 3 weeks post SO injection (3wpi) in the DMSO group, whereas maprotiline treatment significantly increased GCC thickness (FIG. 5b,c). Histological analysis of post-mortem retina wholemounts and semi-thin ON sections consistently demonstrated significantly greater RGC soma and axon survival in the maprotiline group than in the DMSO group (FIG. 5d,e). We confirmed the axon protection effect of maprotiline in the SOHU glaucoma model by TEM analysis of ON cross sections and CTB tracing in wholemount ONs (FIG. 13).

The clinical significance of neuroprotection depends on preserving neuronal function. Therefore, we also investigated whether maprotiline preserves visual function in the glaucomatous mice. The optokinetic tracking response (OKR) is a natural reflex that objectively assesses mouse visual acuity. Another important electrophysiological assessment of RGC function is the pattern electroretinogram (PERG), in which the ERG responses are stimulated with contrast-reversing horizontal bars alternating at constant mean luminance. We used both techniques, which are well established in our lab, to evaluate maprotiline's effect on glaucomatous eyes. Consistent with our morphological and histological results, maprotiline significantly preserved visual function in glaucomatous eyes, as demonstrated by improved amplitude of PERG (FIG. 5f) and visual acuity (FIG. 5g) compared to the DMSO control group. Taken together, these results show that maprotiline treatment achieves significant RGC and ON neuroprotection and preserves visual functions in a mouse glaucoma model, confirming its use as a neuroprotectant.

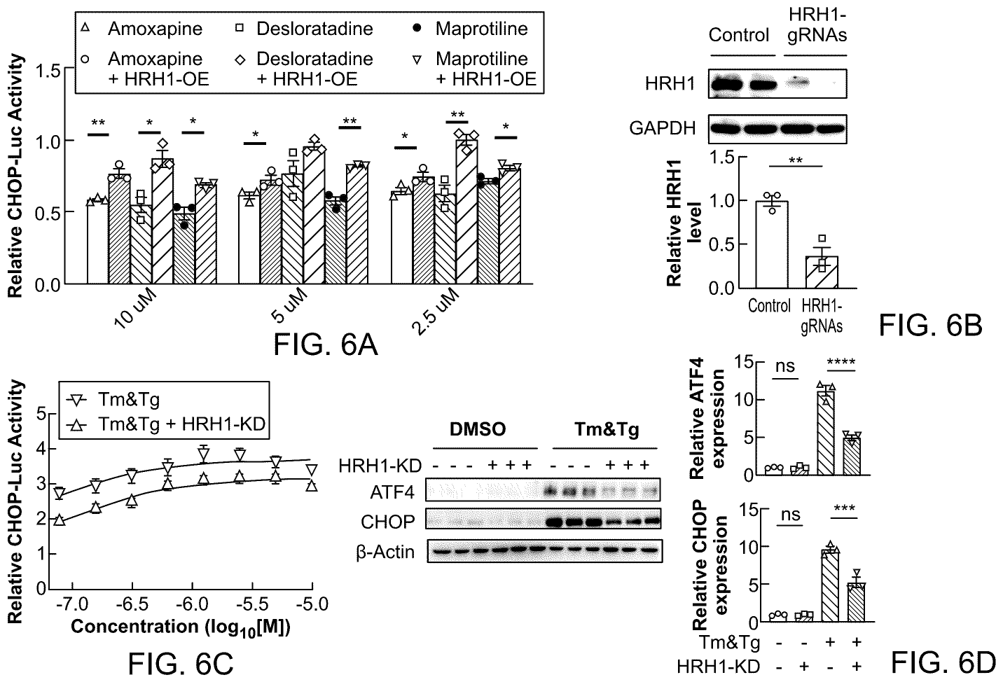

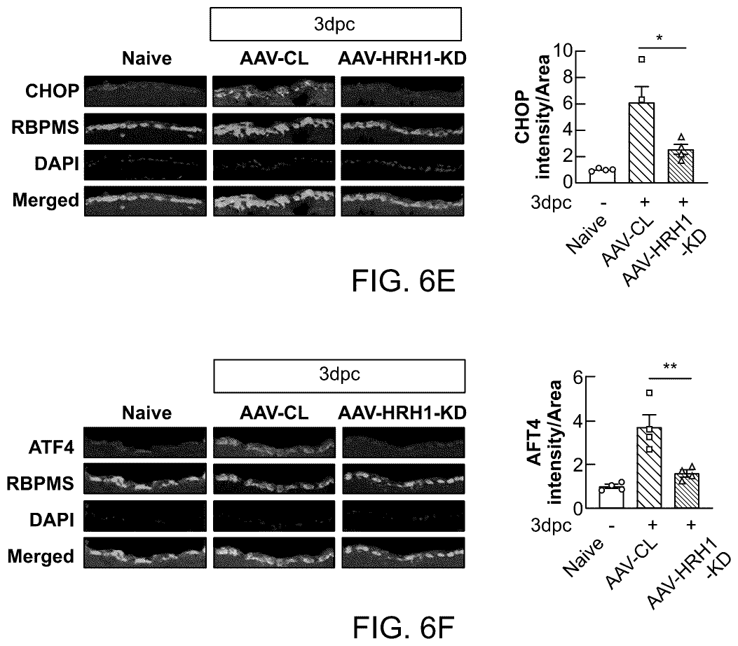

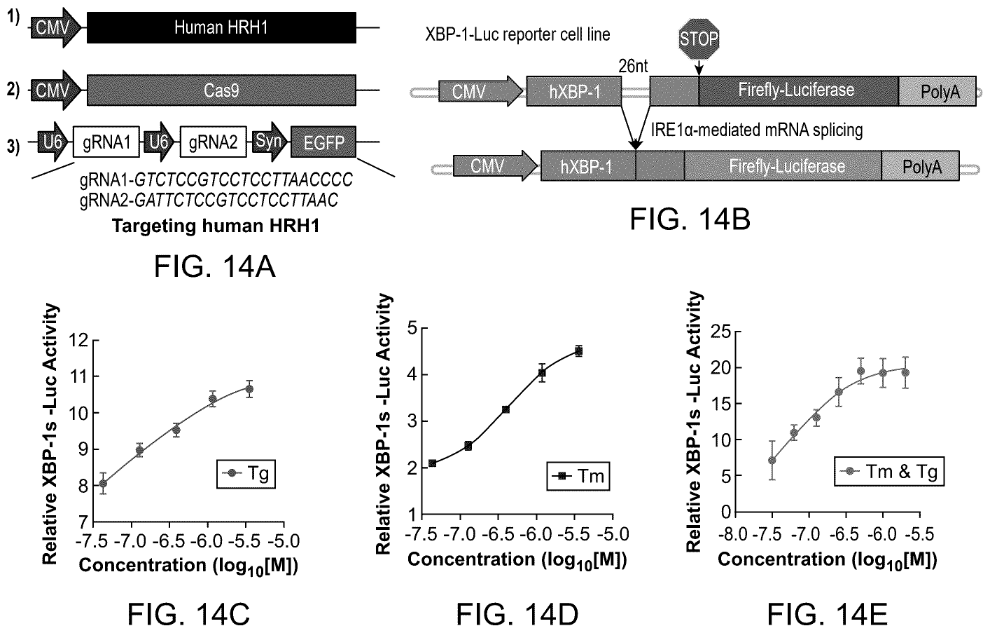

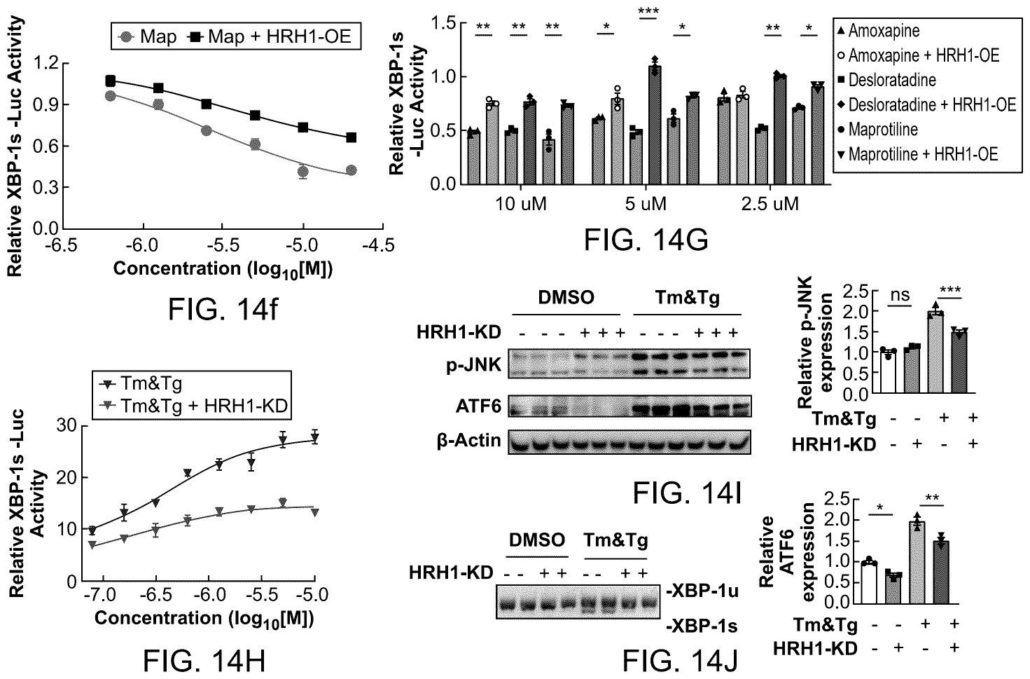

Histamine receptor H1 (HRH1) is a common antagonist target of amoxapine, desloratadine, and maprotiline for ER stress modulation. We next explored potential downstream effectors of the hit compounds for ER stress modulation and neuroprotection. We reasoned that since the three ER stress modulators, amoxapine, desloratadine, and maprotiline, have similar chemical structures, they may act on a common downstream target to restore ER homeostasis. Because, intriguingly, all three agents are potent antagonists of HRH1 with high binding affinities, we hypothesized that HRH1 inhibition may mediate the effects of these compounds on ER stress modulation. To test this hypothesis, we first overexpressed (OE) human HRH1 (FIG. 14a) in the CHOP-Luc reporter cell line by transient transfection. The overexpression of HRH1 significantly but not completely reversed the three compounds' inhibitory effect on Tm/Tg induced CHOP expression (FIG. 6a), indicating that other mechanisms in addition to HRH1 inhibition may also contribute to the compounds' effects on ER stress. To test whether other UPR pathways are also modulated by HRH1, we generated a stable XBP-1-Luc HEK293T reporter cell line expressing the human XBP-1 fragment-fused luciferase construct (FIG. 14b). This construct contains a 26nt intron sequence that will be removed from the mRNA by IRE1α upon ER stress. Splicing of the 26 nt intron will allow a shift of the open reading frame in the mRNA to express luciferase, which will serve as a reporter for the activation of IRE1α pathway. This cell line shows consistent dose response to Tg and Tm (FIG. 14c-e). Maprotiline also shows a dose-dependent inhibition of XBP-1 splicing induced by Tm/Tg. Overexpression of HRH1 reversed the inhibitory effects of maprotiline (FIG. 14f) and amoxapine and desloratadine (FIG. 14g) on XBP-1 splicing. Therefore, overexpression of HRH1 significantly blocked the activities of the three ER stress modulators, consistent with their antagonistic effect on this receptor.

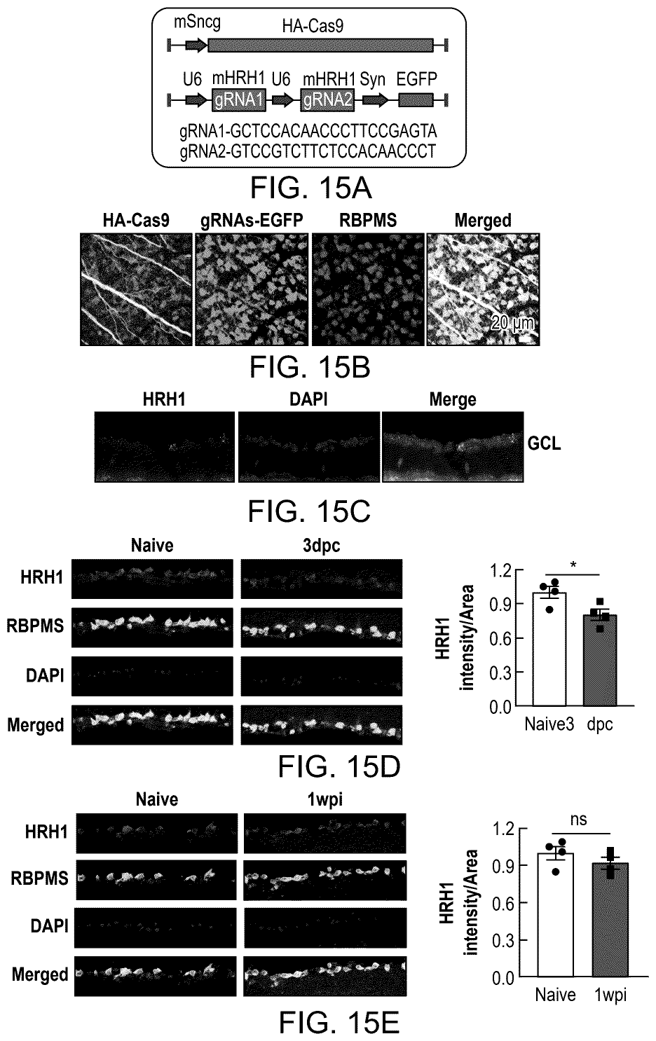

We next tested whether blocking HRH1 itself has a similar ER stress modulation effect as the three compounds. First, we generated a pair of gRNAs targeting human HRH1 (FIG. 14a) and confirmed the HRH1 knockdown (KD) effect of CRISPR in HEK293T cells (FIG. 6b). We then transfected Cas9 and HRH1 gRNAs into the two reporter cell lines and compared Tm/Tg-induced ER stress with or without HRH1 KD. HRH1 inhibition consistently downregulated Tm/Tg-induced CHOP expression (FIG. 6c) and XBP-1 splicing (FIG. 14h). To further confirm the HRH1 KD effect on ER stress, we also examined the protein levels of various UPR molecules and again found that HRH1-KD significantly inhibited Tm/Tg-induced ATF4 and CHOP expression (FIG. 6d); JNK phosphorylation and ATF6 expression (FIG. 14i); and XBP-1 mRNA splicing (FIG. 14j). We previously demonstrated AAV-mSncg promoter mediated Cas9 expression and CRISPR-mediated gene KD in RGCs in vivo. Using the same strategy, we designed gRNAs targeting mouse HRH1 and injected the mixture of AAV-mSncg-Cas9+AAV-mouse HRH1-gRNAs (FIG. 15a) or AAV-control gRNAs intravitreally into mouse eyes (FIG. 15b). The endogenous HRH1 mRNA level was detected in some mouse RGCs (FIG. 15c). Immunostaining showed HRH1 protein levels to be more extensive in RGCs, and crush injury, but not glaucoma, decreased protein expression in RGCs (FIG. 15d,e). AAV-mediated CRISPR KD of HRH1 significantly blocked CHOP and ATF4 expression induced by ONC injury (FIG. 6e, f). Taken together, our studies demonstrated that the three hit compounds inhibit ER stress through their antagonistic effects on their common target, HRH1, suggesting that HRH1 inhibition may provide a potential neuroprotection strategy.

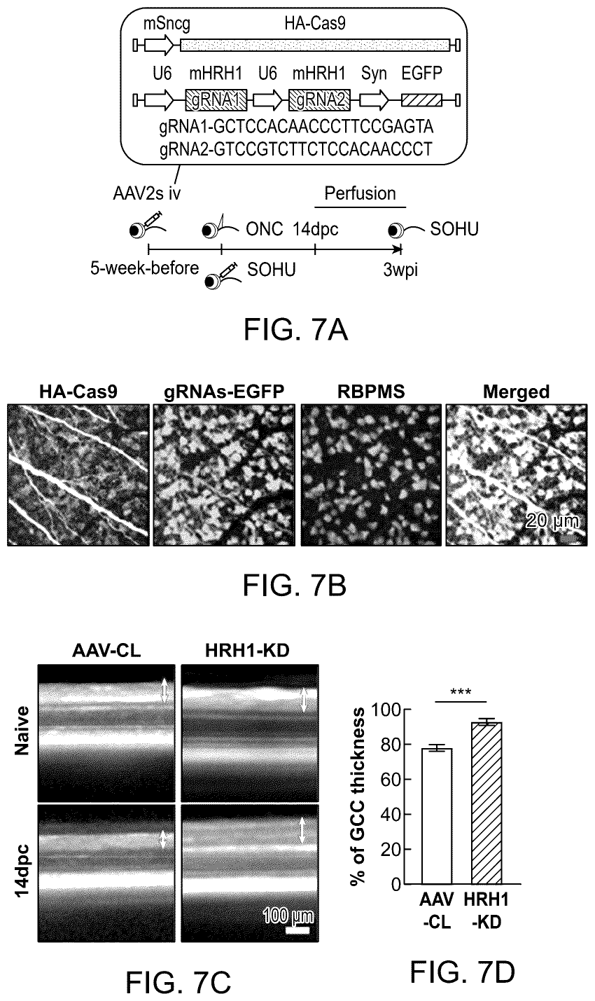

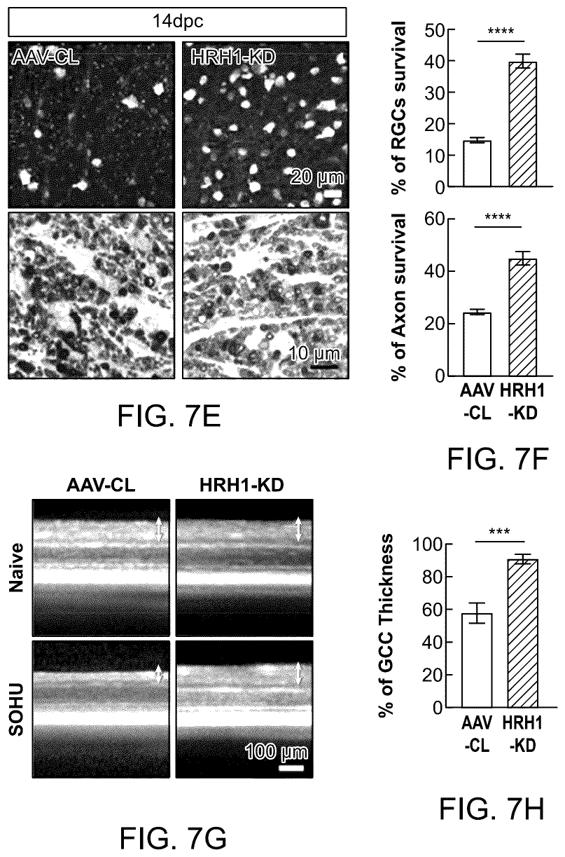

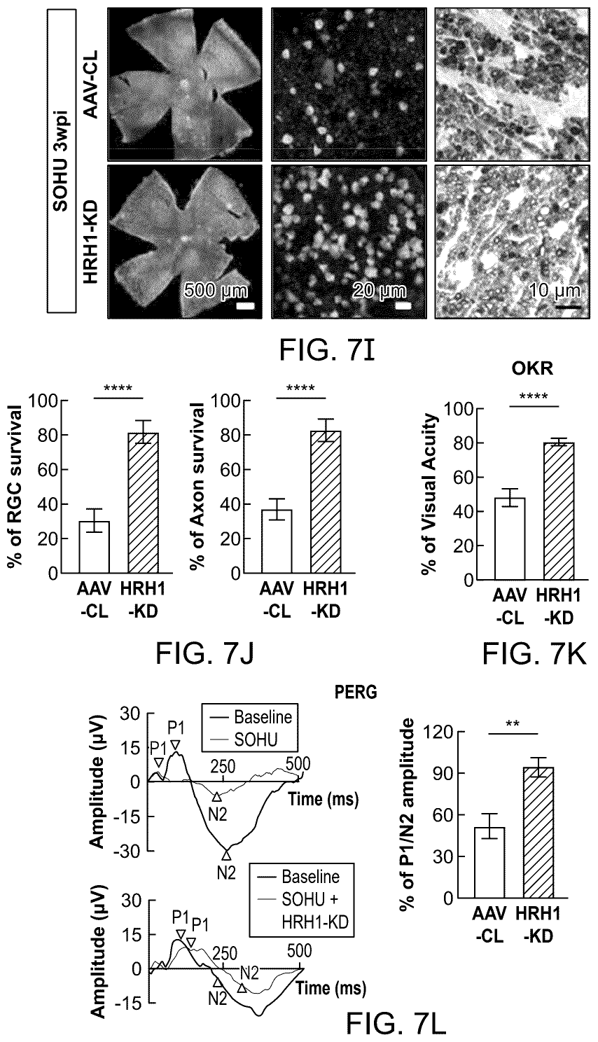

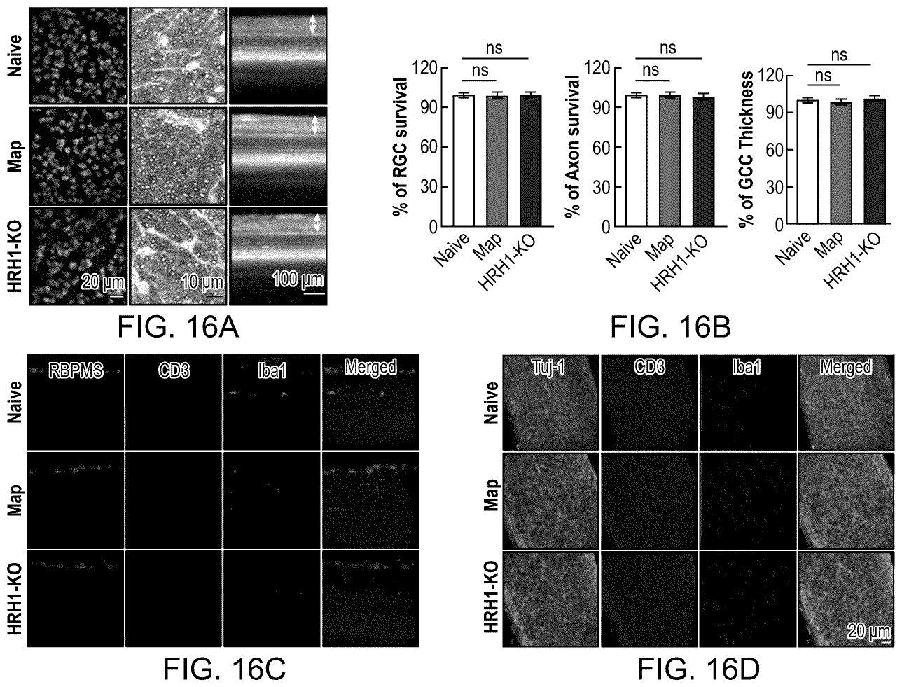

HRH1 KD provides significant neuroprotection in two mouse optic neuropathy models. We next investigated whether HRH1 inhibition also furnishes neuroprotection in two mouse optic neuropathy models. We injected the mixture of AAV-mSncg-Cas9+AAV-mouse HRH1-gRNAs or AAV-control gRNAs intravitreally into one of a mouse's eyes five weeks before ONC (for traumatic ON injury model) or SO intracameral injection (SOHU glaucoma model) and used the contralateral eye as sham control (FIG. 7a). In the ONC model, in vivo OCT imaging showed that the GCC was significantly thicker in HRH1 KD mice than control mice (FIG. 7b,c). Histological analysis of post mortem retina wholemounts and ON sections consistently demonstrated significant protection of RGC somata and axons by HRH1 KD (FIG. 7d,e). In the SOHU glaucoma model, HRH1 KD also showed a significantly thicker GCC and greater survival of RGC somata and axons than controls (FIG. 7f-i). Importantly, we also confirmed visual function preservation by HRH1 KD, measured by OKR and PERG (FIG. 7j,k). Therefore, like maprotiline treatment, blocking HRH1 significantly protects RGCs and ONs and preserves visual functions in two mouse optic neuropathy models, indicating the promising therapeutic potential of HRH1 inhibition in traumatic and glaucomatous neurodegeneration. Investigation of the long-term safety of maprotiline and HRH1 KD on naïve mouse retinas revealed no RGC or ON degeneration one month after systemic maprotiline administration or three months after local retina AAV-mediated CRISPR HRH1 KD (FIG. 16a,b). We also found no immune cell infiltration in retina and ON after either of these treatments (FIG. 16c-f).

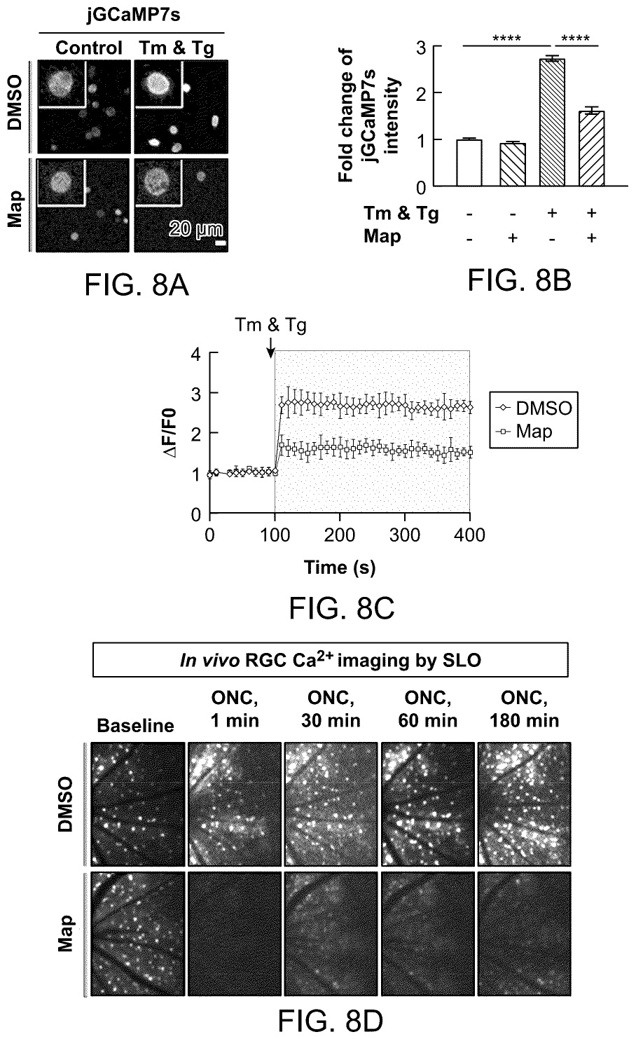

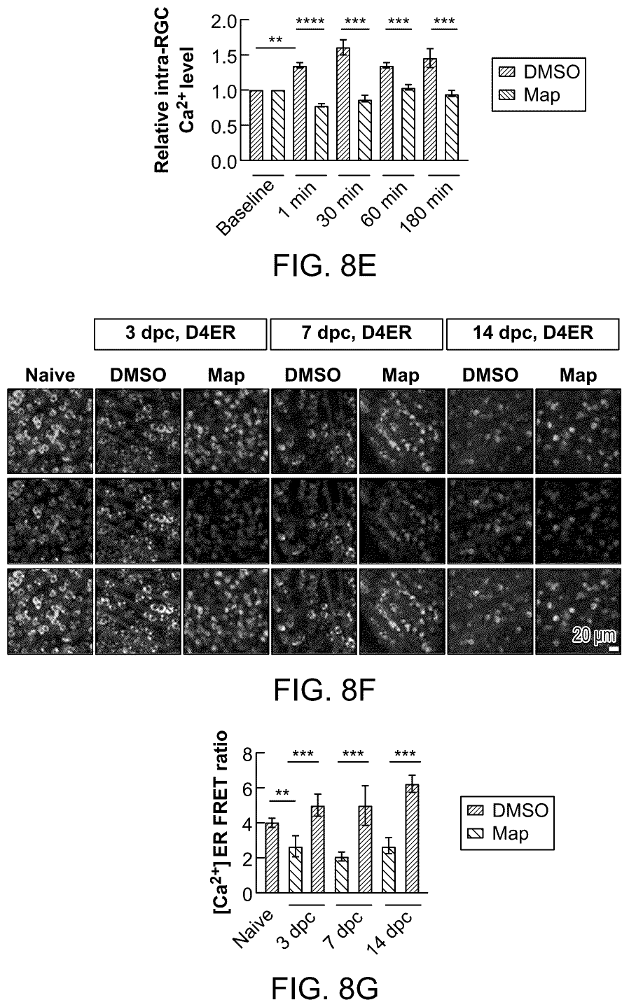

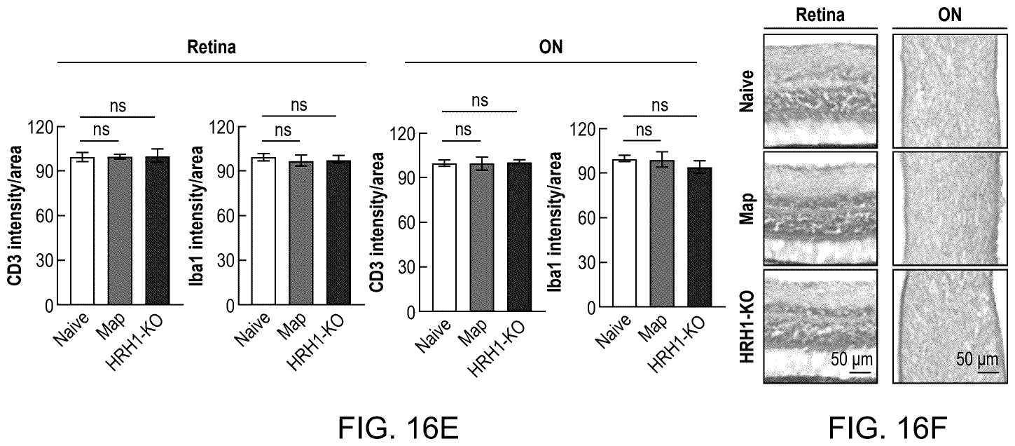

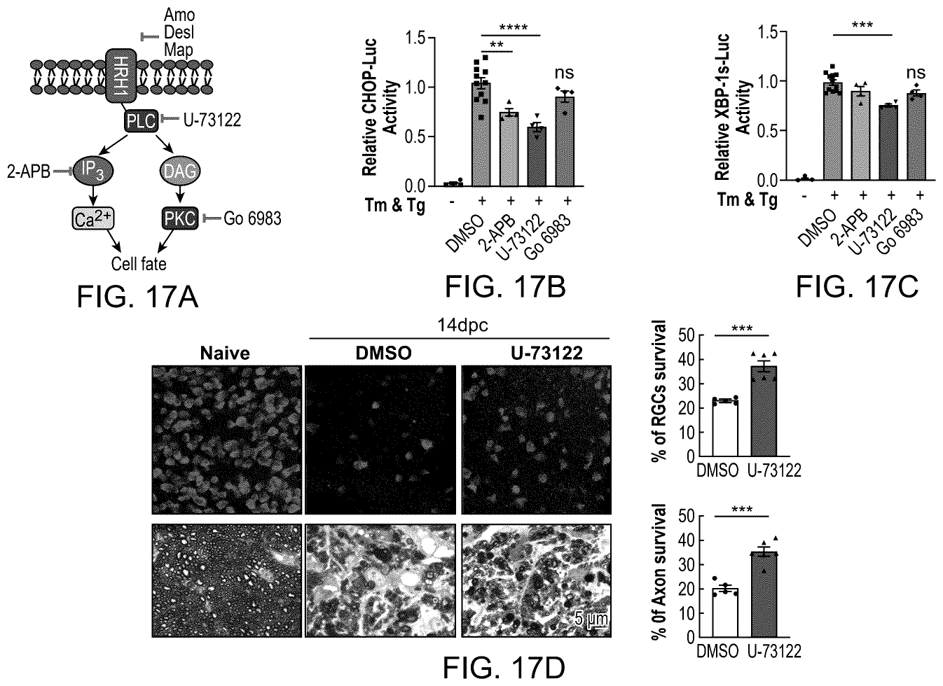

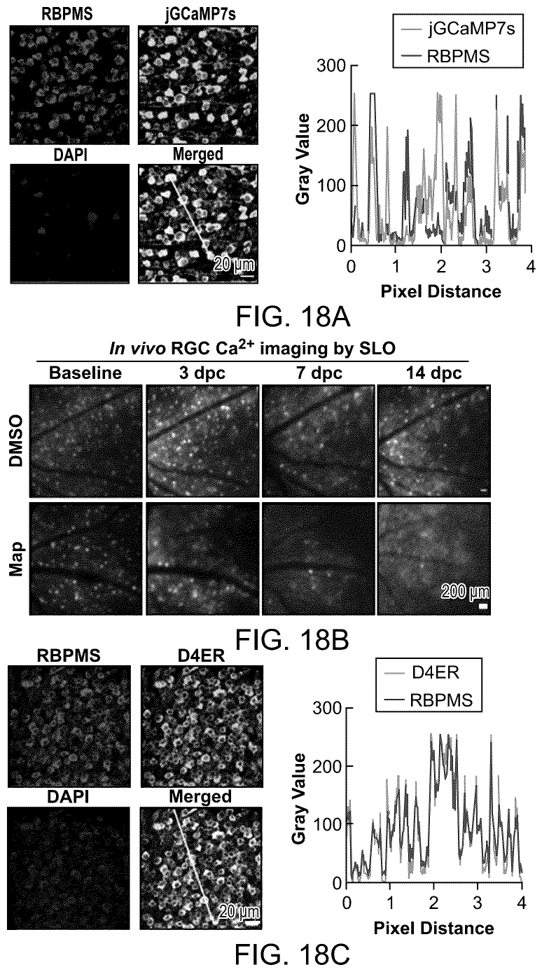

Maprotiline blocks axon injury-induced intracellular Ca^{2 +} influx by inhibiting HRH1-mediated ER Ca^{2 +} release. HRH1 is a Gq protein-coupled receptor that can activate phospholipase C (PLC)-IP₃ pathway; it leads to ER Ca^{2 +} release through IP3 receptors and cytosol Ca^{2 +} influx (FIG. 17a). ON injury is well-known to induce rapid intra-axonal Ca^{2 +} influx that leads to axon degeneration. Ca^{2 +} release from the ER, the major intracellular Ca^{2 +} storage site, contributes to the deleterious intra-axonal Ca^{2 +} influx, and at the same time, the disturbance of ER Ca²⁺ homeostasis is also an important initiator of the ER stress. We reasoned that maprotiline may block ER Ca^{2 +} release by inhibiting HRH1, and therefore restore ER Ca^{2 +} homeostasis and prevent ER stress. Using pharmacologic small molecule inhibitors of signaling downstream of HRH1, we confirmed that blocking PLC or IP₃, but not DAG-PKC, decreased CHOP and XBP-1 activation induced by Tm/Tg (FIG. 17b,c), suggesting that HRH1-mediated ER Ca^{2 +} release contributes to ER stress. Moreover, PLC inhibitor U-73122 protected RGC somata and axons in vivo after ONC injury (FIG. 17d). Therefore, we investigated the effect of maprotiline on the intracellular and ER Ca^{2 +} levels. First, we transfected genetically encoded Ca^{2 +} sensor jGCaMP7s into HEK293T cells (FIG. 8a), and confirmed that maprotiline significantly blocked Tm/Tg-induced intracellular Ca^{2 +} influx (FIG. 8b,c). Next, we assessed Ca^{2 +} influx in RGCs after ONC in vivo: we confirmed efficient AAV-mediated jGCaMP7s expression in RGCs (FIG. 18a); and then recorded in vivo RGC Ca^{2 +} imaging in living animals by scanning laser ophthalmoscope (SLO) at different time points after ONC injury. Within minutes after ONC, intra-RGC Ca^{2 +} levels were significantly elevated, indicating rapid Ca^{2 +} influx induced by axon injury, whereas maprotiline significantly decreased intra-RGC Ca^{2 +} levels at both early time points (FIG. 8d,e) and later time points (FIG. 18b). This significant decrease indicates efficient blocking of Ca^{2 +} influx by maprotiline, presumably through inhibition of HRH1-mediated Ca^{2 +} release from the ER. To definitively prove this mechanism, we measured intra-ER Ca^{2 +} levels by expressing a FRET-based ER Ca²⁺ sensor, D4ER, driven by the mSncg promoter, in mouse RGCs specifically (FIG. 18c). We confirmed that ONC significantly depleted ER Ca^{2 +} of RGCs, whereas, in dramatic contrast, maprotiline maintained ER Ca^{2 +} concentration at much higher levels (FIG. 8f,g). Taken together, our data demonstrated that maprotiline blocks ER Ca^{2 +} release through HRH1 inhibition, by which it restores ER homeostasis, prevents deleterious intracellular Ca^{2 +} influx and ultimately protects injured/diseased RGCs and ONs.

The present experiments first identified three FDA approved medicines, two antidepressants (amoxapine and maprotiline) and one antihistamine/anti-allergy drug (desloratadine), as potent blockers of ER stress-induced CHOP expression, and then as general modulators of all three UPR pathways and as effective neuroprotectants. We favor a model in which they have a global effect inhibiting the UPR by modulating upstream signaling of ER stress. Indeed, this notion receives support from our finding that inhibition of HRH1, a common antagonistic target of all three drugs, also achieved comparable ER stress inhibition and in vivo neuroprotection in both traumatic ON injury and ocular hypertension glaucoma models. It is known that HRH1 activation leads to Ca^{2 +} release from ER and that depletion of ER Ca^{2 +} worsens ER function and induces ER stress. Using both cytosol and ER-targeted Ca^{2 +} biosensors, we found that maprotiline inhibits ER inducer (Tm/Tg)-induced and axon injury-induced ER Ca²⁺ release and cytosol Ca^{2 +} influx both in cultured cells and mouse RGCs in vivo. The restoration of Ca^{2 +} homeostasis then attenuates the global UPR signaling. We found maprotiline to be the most potent of the three drugs in modulating ER stress based on in vitro cell-based assays and that its systemic administration caused no detectable toxicity on the normal retina, but significantly protected RGCs and ONs and visual functions in mouse disease models of glaucoma and traumatic injury. The potent in vivo neuroprotection of maprotiline correlates with its potent ER stress modulation, further evidence for its on-target mechanism of action.

Our studies not only identified potent ER stress modulators and effective neuroprotectants, but also revealed a molecular mechanism that regulates ER stress: maprotiline (possibly amoxapine and desloratadine as well) restores ER homeostasis and keeps the three UPR pathways in check by blocking HRH1-mediated Ca^{2 +} release from the ER. Many small molecule modulators of ER stress that target the signaling molecules in the three main UPR pathways have been developed for different purposes, some of them are neuroprotective but none target intracellular Ca^{2 +} signaling, which is critical for many neurodegenerative diseases associated with axon degeneration. Other HRH1 antagonists may also be able to modulate ER stress and therefore merit further exploration as neuroprotectants. However, some of the other HRH1 antagonists that we tested inhibited ER stress only at high concentration (FIG. 1d, FIG. 9e), and overexpression of HRH1 only partially blocked the three hit compounds' effect (FIG. 6a).

These results suggest that other mechanisms in addition to HRH1 may also be responsible for amoxapine/desloratadine/maprotiline-mediated ER stress inhibition, possibly through other receptors that are also modulated by these compounds. Our in vivo findings establish maprotiline as a candidate neuroprotectant and HRH1 as a therapeutic target for glaucoma, and possibly for neurodegenerative diseases more generally. Because their safety profiles, pharmacokinetics and pharmacodynamics, including penetration of blood-brain barrier, are well known, and because of their extensive clinical usage, maprotiline and/or HRH1 inhibition is a promising pharmacological approach for neuroprotection that can be readily translated to pre-clinical studies in large animals and evaluation in human patients. To this end these compounds are even more attractive if sustained, local delivery to the eye is pursued, thereby minimizing the potential for systemic side effects.

In this study, we constructed two ER stress reporter HEK293T cell lines that stably express human CHOP promoter-driven luciferase or fused human XBP-1 fragment-luciferase containing a 26nt intron sequence that will be removed upon ER stress and IREα activation to allow luciferase expression. Through a small scale HTS with the CHOP-Luc reporter line and further validation with both CHOP-Luc and XBP-1-Luc reporter lines, we demonstrated that this powerful strategy efficiently identifies ER stress modulators. We previously found that the two ER stress molecules, CHOP and XBP-1, play opposing roles in glaucomatous degeneration: deletion of CHOP and activation of XBP-1 protect diseased RGCs and ON synergistically. A more selective blocker of the PERK-eIF2α-ATF4 pathway, such as ISRIB, may be more valuable, although ISRIB did not have a significant inhibition effect with our luciferase reporter cell line driven by a human CHOP promoter (FIG. 1d). It showed significant neuroprotection in the ONC model (FIG. 4), indicating low sensitivity of the reporter line. The much larger murine CHOP promoter (8.5 kb) in the CHO cell line may be more sensitive to compounds or have more cis-regulatory components than the human CHOP promoter (˜1 kb) in our HEK cell line, but they do share similar “hits”, including GSK2606414 and trifluoperazine. Cross-checking hit compounds with these two reporter lines will be worthwhile to further confirm CHOP inhibitory effects.

In summary, we identified three FDA approved drugs as potent ER stress modulators and effective neuroprotectants through a small scale HTS. We found that both systemic administration of maprotiline and locally applied CRISPR-mediated RGC-specific HRH1 inhibition achieve significant neuroprotection and visual function recovery in in vivo mouse models of glaucoma and traumatic ON injury. Based on the demonstration of their molecular target and mechanism that we provide in this report and their well-established safety, pharmacological and clinical usage profiles, maprotiline, its structural analogs, and HRH1 antagonists are useful for thorough pre-clinical and clinical evaluation as neuroprotectants.

### Methods

Animals. C57BL/6J WT (#000664) mice (7-9 weeks old, male) were purchased from Jackson Laboratories (Bar Harbor, Maine) and housed in standard cages on a 12-hour light-dark cycle with room temperature at 25±2° C. and humidity between 40 and 60%. All experimental procedures were performed in compliance with animal protocol approved by the IACUC at Stanford University School of Medicine.

Constructs. The phCHOP-Luciferase (−954) construct containing human CHOP promoter driven-luciferase was originally made by Dr. Pierre Fafournoux and given by Dr. Shigeru Takahashi. The XBP1-Luciferase construct was from Dr. Albert Koong, containing the luciferase gene fused downstream of an XBP1 fragment containing the 26 nt intron, splicing of the intron by IRE1 under ER stress results in a frameshift and luciferase translation. The coding regions of D4ER (a gift from Dr. Paola Pizzo, Department of Biomedical Sciences, University of Padua, Italy) and jGCaMP7s (Addgene, #104487) were cloned into our pAM-AAV-mSncg-WPRE backbone containing the RGC-specific mSncg promoter. The human HRH1 coding sequence was cloned from HEK293T cell genomic DNA and inserted into a backbone containing the CMV promoter to create a CMV-HRH1 vector. The AAV2-mSncg-Cas9 and the AAV-U6-sgRNAs-Syn-EGFP have been described before. The mouse HRH1 gRNA sequences are: gRNA1 (5′-GCTCCACAACCCTTCCGAGTA-3′) and gRNA2 (5′-GTCCGTCTTCTCCACAACCCT-3′). The human HRH1 gRNA sequences are: gRNA1 (5′-GTCTCCGTCCTCCTTAACCCC-3′) and gRNA2 (5′-GATTCTCCGTCCTCCTTAAC-3′).

AAV production and intravitreal injection. AAV2 vector was co-transfected with the pHelper plasmid (Stratagene) and pAAV2 (pACG2)-RC triple mutant into HEK293T cells for 72 hours before purification with polyethylene glycol and cesium chloride density gradient centrifugation. The AAV titers were determined by real-time PCR and diluted to 1.5×10¹² vector genome (vg)/ml for mouse intravitreal injection. For intravitreal injection, mice were anesthetized by xylazine and ketamine based on their body weight (0.01 mg xylazine/g+0.08 mg ketamine/g). A pulled and polished microcapillary needle was inserted into the peripheral retina just behind the or a serrata. Approximately 2 μl of the vitreous was removed to allow injection of 2 μl AAV into the vitreous chamber to achieve 3×10⁹ vg/retina.

ER stress reporter cell lines. HEK293T cells were transiently co-transfected with phCHOP-Luciferase or XBP-1-Luciferase with pEGFP-puro using Lipofectamine 2000 (Invitrogen, Carlsbad, CA) at ratio 5:1 to ensure EGFP positive cells are also luciferase construct positive and puromycin resistant. After a serial selection with puromycin and EGFP expression, multiple stably expressing clones (CHOP-Luc/puro or XBP-1-Luc/puro) were isolated by a serial dilution. After expansion of individual clones, one CHOP-luciferase stable line and one XBP-1-luciferase line were selected based on their responses to Tm/Tg treatment, and maintained by puromycin as stable reporter cell lines used in this study.

Cell culture and viability assays. HEK293T cells were grown in Dulbecco's Modified Eagle's Medium (DMEM) (Invitrogen, 11995081) supplemented with 100 μg/mL streptomycin and 100 units/mL penicillin (Gibco, 15140122), and 10% fetal bovine serum (FBS) (Invitrogen, 10437-028). Cells were maintained under standard tissue culture conditions (5% CO₂, 37° C.). PolyJet™ (SignaGen Laboratories, SL100688) transfection reagent was used for transient cell transfection. For detection of relative cellular viability levels, cells (10000/well) were seeded into poly-d-lysine coated 96-well plates (Falcon, 353072) and treated as described. Then, MTT (5 mg/mL, 10 μL/well) (MedChemExpress, HY-15924) was added. After incubation at 37° C. for 3-4 hours, DMSO was added to dissolve the precipitate, and the absorbances were determined at 570 nm by a Tecan Infinite M1000 Pro plate reader.

Cell-based HTS. The HTS to identify CHOP expression inhibitors was performed at the Stanford High-Throughput Bioscience Center with small molecule libraries containing 4846 total known bioactive, FDA approved drugs and clinical trial compounds; libraries included Biomol FDA, Biomol ICCB, Microsource (MS) Spectrum, Sigma LOPAC, and NIH Clinical Collection (NIH-CC). All the compounds were run in 7-point dose response based on a previous publication; except the NIH Clinical collection (446 compounds) which were tested in duplicate due to the small quantity of compounds on hand. Most of the NIH-CC was screened at 10 UM, but this varied per compound. The Z′ of the assay was 0.5, details on instrumentation can be found here: https://med.stanford.edu/htbc/equipment/liquid.html. We used the Pin Tool to transfer 100 nL of the compounds into 50 μL final volume. Therefore, we did a 500-fold dilution of the stock plates, which were at 10, 5, 2.5, 1.25, 0.625, 0.3125, and 0.156 mM resulting in final concentrations of 20, 10, 5, 2.5, 1.25, 0.625, and 0.3125 UM (for most of the compounds but not all). The final DMSO concentration was 0.2% for all wells. Briefly, CHOP-Luc cells were seeded in 384-well plates (Greiner Bio-One CELLSTART) at a density of 2×10⁵ cells per well in 40 μL medium and cultured for 24 hours before treatment with tunicamycin (Tm)+thapsigargin (Tg) at 1 μM in 10 μL medium followed by adding 100 nL of one of the testing compounds using a Staccato SciClone ALH3000 small molecule liquid handling system (Caliper Life Sciences) and V&P Scientific 384 pin tools. AeraSeal sterile adhesive microplate seals (Excel Scientific; Victorville, CA) were used to seal plates that were incubated at 5% CO2, 37° C. for 24 hours, when luciferase activity was assayed by adding 10 μL of Bright™-Glo Luciferase reagent (Promega) to each well and detected by a Tecan Infinite M1000 Pro plate reader. Percentage of CHOP-Luc inhibition=100%−(Compound value−DMSO control value)/(Tm/Tg value−DMSO control value)×100%. The compounds achieving >30% of CHOP-Luc inhibition were considered as “hits”. Luciferase activity of all the tested compounds was determined by meta-analysis of luciferase activity in different cell lines under the control of a generic or other promoters. Any compound that appeared in more than 3 non-related luciferase screens was likely a toxic compound or luciferase inhibitor and therefore eliminated. There was no true cutoff, other than an IC₅₀<20 UM (or the highest concentration tested). Most of these compounds were toxic, but we did not specifically examine toxicity because our goal here was to eliminate the non-specific hits regardless of whether they were luciferase inhibitors or toxic compounds.

Commercial Compounds. Thapsigargin (Sigma, T9033), Tunicamycin (Sigma, T7765), Maprotiline (Sigma, M9651), Amoxapine (MedChemExpress, HY-B0991), Desloratadine (MedChemExpress, HY-B0539), Desipramine (Sigma, D3900), Trifluoperazine (Sigma, T8516), Clomipramine (Sigma, C7291), Amitriptyline (Sigma, A8404), Quetiapine (Sigma, Q3638), Olanzapine (MedChemExpress, HY-14541), Doxepin (MedChemExpress, B078), Loxapine (Sigma, L106), Norquetiapine (Sigma, 07849), dimethyl sulfoxide (DMSO) (Sigma, D8418), 2-APB (MedChemExpress, HYW009724), U-73122 (MedChemExpress, HY13419), Go 6983 (MedChemExpress, HY13689), GSK2606414 (Sigma, 516535), ISRIB (MedChemExpress, HY-12495).

Protein Preparation and Immunoblotting. Cell and tissue lysates were prepared in RIPA buffer (Themo Fisher Scientific, 89901) and supplemented with Halt Protease inhibitor cocktail (Themo Fisher Scientific, PI78437). The total protein concentration of lysates was measured by the Pierce BCA Protein Assay Kit (Thermo Fisher Scientific, 23227). 20 μg protein of each sample were denatured at 95° C. for 15 min in 100 mM DTT+1× Laemmli buffer before being separated by SDS-PAGE. We then transferred the protein samples to 0.2 μm nitrocellulose membranes (Bio-Rad, 1610097) and blocked with 5% BSA for 2 hours. Subsequently, the membranes were incubated with primary antibodies (1:1000) overnight at 4° C. After washing in TBST, these membranes were incubated with horseradish peroxidase-conjugated secondary antibodies (Cell Signaling, 7074S and 7076S, 1:1000) and visualized using a GE-A1600 imaging system. Images were quantified with ImageJ software. The primary antibodies used were PERK (Cell Signaling, 3192S), p-PERK (Cell Signaling, 3179S), eIF2a (Cell Signaling, 5324S), p-eIF2a (Cell Signaling, 3597L), ATF4 (Cell Signaling, 11815S), CHOP (Cell Signaling, 2895S), ATF6 (Cell Signaling, 65880T), IRE1a (Cell Signaling, 3294T), IRE1 (phosphor-S724) (Thermo Fisher, PA116927), phospho-SAPK/JNK (Thr183/Tyr185) (Cell Signaling, 9251S), phospho-p38 MAPK (Thr180/Tyr182) (Cell Signaling, 9215S), β-Actin (sigma, A5441), XBP-1s (Biolegend, 647502), XBP-1 (Santa Cruz, sc-8501), anti-RBPMS (Custom made at ProSci Inc).

RT-PCR for XBP1 splicing assay and Q-PCR for ER stress genes. Cells were plated into poly-d-lysine coated 6-well plates (Fisher, 353046) and treated as indicated at 37° C. with 5% CO₂. The total RNA was isolated using Trizol (Thermo Fisher Scientific, 10296010) and 500 ng RNA was reverse transcribed using the High-Capacity cDNA Reverse Transcription Kit (Thermo Fisher Scientific, 4374966) to acquire total cDNA. The XBP-1 mRNA splicing primers (forward primer SEQ ID NO:1 5′-GGGGCTTGGTATATATGTGG-3′, reverse primer SEQ ID NO:2 5′-CCTTGTAGTTGAGAACCAGG-3′) were utilized to amplify the XBP-1 amplicon containing the 26nt intron that will be released by IRE1α upon ER stress, with Q5 High-Fidelity DNA Polymerase (NCB, M0491L). PCR products (2 μg) were resolved on 2.5% agarose gels, visualized with GelRed (Biotium), and quantified by ImageJ (NIH). The relative mRNA expression levels of target genes were detected by PowerUP SYBR Green Master Mix (Thermo Fisher, A25776) and C1000 TOUCH CYCLER w/48W FS RM PCR System (Bio-rad, 1851148). Thermal cycles were 95° C. for an initial 5 min followed by 40 cycles of denaturation at 95° C. for 15 s, annealing at 60° C. for 30 s and extension at 72° C. for 60s. Transcripts were normalized to GAPDH and all measurements were performed in triplicate. Primers used were: GAPDH, forward SEQ ID NO:3 5′-GTCTCCTCTGACTTCAACAGCG-3′ and reverse SEQ ID NO:4 5′-ACCACCCTGTTGCTGTAGCCAA-3′; ATF6, forward SEQ ID NO:5 5′ TGGAAGCAGCAAATGAGACG-3′ and reverse SEQ ID NO:6 5′-TGAGGAGGCTGGAGAAAGTG-3′; ERN1, forward SEQ ID NO:7 5′-CGAACGTGATCCGCTACTTC-3′ and reverse SEQ ID NO:8 5′ ATGTTGAGGGAGTGGAGGTG-3′; EIF2AK3, forward SEQ ID NO:9 5′-GTCCCAAGGCTTTGGAATCTGTC-3′ and reverse SEQ ID NO:10 5′-CCTACCAAGACAGGAGTTCTGG-3′; CHOP, forward SEQ ID NO:11 5′-ACCAAGGGAGAACCAGGAAACG-3′ and reverse SEQ ID NO:12 5′-TCACCATTCGGTCAATCAGAGC-3′; ATF4, forward SEQ ID NO:13 5′-GTCCCTCCAACAACAGCAAG-3′ and reverse SEQ ID NO:14 5′-TGTCATCCAACGTGGTCAGA-3′; DNAJC3, forward SEQ ID NO:15 5′-GCCTGCATCTGCTTTATGCT-3′ and reverse SEQ ID NO:16 5′-TCTGCAAGGCTGTGAAGAGA-3′; GADD45a, forward SEQ ID NO:17 5′-GGAGGAAGTGCTCAGCAAAG-3′ and reverse SEQ ID NO:18 5′-ACATCTCTGTCGTCGTCCTC-3′; DNAJB9, forward SEQ ID NO:19 5′-GGAAGGAGGAGCGCTAGGTC-3′ and reverse SEQ ID NO:20 5′-ATCCTGCACCCTCCGACTAC-3′; Calreticulin, forward SEQ ID NO:21 5′-CGATGATCCCACAGACTCCA-3′ and reverse SEQ ID NO:22 5′-CCGTCCATCTCTTCATCCCA-3′; Bip, forward SEQ ID NO:23 5′-GCCTGTATTTCTAGACCTGCC-3′ and reverse SEQ ID NO:24 5′-TTCATCTTGCCAGCCAGTTG-3′; XBP-1s, forward SEQ ID NO:25 5′-CTCCAGAGACGGAGTCCAAG-3′ and reverse SEQ ID NO:26 5′-CACCTGCTGCGGACTC-3′.

ON crush model. The ON was exposed intraorbitally while care was taken not to damage the underlying ophthalmic artery, and crushed with a jeweler's forceps (Dumont #5; Fine Science Tools, Foster City, California) for 5 seconds approximately 0.5 mm behind the eyeball. Eye ointment containing neomycin (Akorn, Somerset, New Jersey) was applied to protect the cornea after surgery. For compound treatment, each eye received intravitreal injection with 2 μl of 2 mM test compounds once and intraperitoneal (i.p.) injection daily (15 mg/kg) for 14 days after ONC. Control groups received the same volume of DMSO as vehicle control.

SOHU glaucoma model and IOP measurement. Mice were anesthetized by an intraperitoneal injection of Avertin (0.3 mg/g) and received the SO (Alcon Laboratories, 1,000 mPa·s) injection at 9-10 weeks of age. Prior to injection, one drop of 0.5% proparacaine hydrochloride (Akorn, Somerset, New Jersey) was applied to the cornea to reduce its sensitivity during the procedure. A 32G needle was tunneled through the layers of the cornea at the superotemporal side close to the limbus to reach the anterior chamber without injuring lens or iris. Following this entry, ˜2 μl silicone oil (1,000 mPa·s, Silikon, Alcon Laboratories, Fort Worth, Texas) was injected slowly into the anterior chamber using a homemade sterile glass micropipette, until the oil droplet expanded to cover most areas of the iris (diameter ˜1.8-2.2 mm). After the injection, veterinary antibiotic ointment (BNP ophthalmic ointment, Vetropolycin, Dechra, Overland Park, Kansas) was applied to the surface of the injected eye. The contralateral control eyes received mock injection with 2 μl normal saline to the anterior chamber. Throughout the procedure, artificial tears (Systane Ultra Lubricant Eye Drops, Alcon Laboratories, Fort Worth, Texas) were applied to keep the cornea moist. For compound treatment, each eye received retrobulbar injection twice on day 0 and day 10 with 50 μl of 2 mM maprotiline, and daily i.p. injection with maprotiline (15 mg/kg) for 3 weeks after SO injection. Control groups received the same volume of DMSO as vehicle control.

The IOP of both eyes was measured by the TonoLab tonometer (Colonial Medical Supply, Espoo, Finland) according to product instructions under a sustained flow of isoflurane (3% isoflurane at 2 L/minute mixed with oxygen) delivered to the nose by a special rodent nose cone (Xenotec, Inc., Rolla, Missouri). 1% Tropicamide Sterile Ophthalmic Solution (Akorn, Somerset, New Jersey) was applied three times at 3-minute intervals to fully dilate the pupils (about 10 minutes) before taking measurements. During this procedure, artificial tears were applied to keep the cornea moist. Since IOP measurement requires pupil dilation, which essentially relieves the ocular hypertension during the period of pupil dilation, we only measure IOP 3 weeks after SO injection immediately before sacrificing the animals in the ND (no dilation) SOHU model that we described before.

LC-MS analysis of maprotiline in retina. Retinal tissues were homogenized with 100 μL of pre-chilled 20% acetonitrile and then diluted 2-fold with blank mouse plasma. An aliquot of 20 μL of diluted retina homogenate was extracted with 100 μL of methanol: acetonitrile (5:95, v: v) containing the internal standard (Verapamil). The mixture was shaken on a shaker for 15 minutes and then centrifuged at 3220 g for 15 minutes. An aliquot of 70 μL of the supernatant was mixed with 70 μL of water for the injection to the LC-MS. Calibration standards and quality control samples were prepared by spiking 2 μL of the test compound into 18 μL of blank mouse plasma, and the resulting plasma was processed with the unknown samples in the same batch. The extracts were analyzed by a Shimadzu LC-30AD interfaced to a Sciex API 5000 system. The extracts were injected onto an ACE 3 C18 column (50×2.1 mm, 3.0 μm) and separated by the gradient elution using water with 10 mM ammonium acetate (A) and acetonitrile with 0.1% formic acid (B) as mobile phases. The gradient program started at 10% B, held for 0.2 min, ramped to 95% B at 1.5 min, remained at 95% at 2.4 min, dropped to 10% B at 2.45 min, and stayed at 10% B till 3.2 min. The mass spectrometer was operated in positive electrospray ionization under the multiple reaction monitoring (MRM) mode for the detection of the maprotiline (278.277-to-250.2 m/z) and the internal standard (455.346-to-165 m/z). The calibration curve fitted by linear regression was used to quantify the analytes in the matrix using Analyst software 1.6.2 (Sciex).

Immunohistochemistry of whole mount and cross sections of retina. After perfusion fixation with 4% PFA in PBS, mice eyeballs and ONs were dissected out and post-fixed with 4% PFA for 2 hours at room temperature. Retinas were dissected out for whole mount retina immunostaining. For cryo-section with Leica cryostat, the eyeballs and ONs were embedded in tissue-tek OCT (Sakura) on dry ice for subsequent cryo-section. The sections were blocked with 10% goat serum (Sigma, G9023) for 2 hours before incubating with primary antibodies: RBPMS 1:4000, others 1:200 overnight at 4° C. After washing 3 times with PBS, samples were incubated with secondary antibodies (1:400; Jackson ImmunoResearch, West Grove, Pennsylvania) at room temperature for 2 hours. Tissues were washed with PBS 3 times before mounting with Fluoromount-G (SouthernBiotech, Alabama). Confocal images were obtained by a Zeiss LSM 800 microscope (Carl Zeiss Microscopy).

Intracellular Ca^{2 +} imaging in HEK293T cells. HEK293T cells were seeded at 10% confluence the day before transfection in poly-D-lysin-coated glass bottom 35 mm dishes (MatTek, P35GC1.510C). Two hours prior to transfection, media was removed and 1 mL of FBS-free DMEM media was added to each well. We then mixed 2 μg of the jGCaMP7s plasmid in 100 μl DMEM. In a separate tube, we diluted 4 μl PolyJet™ (SignaGen Laboratories, SL100688) reagent in 100 μl with DMEM, and incubate for 10 minutes. The complete mixture of DNA and PolyJet was incubated for another 15 min before being added to the HEK293T cells. Six hours later, the media was changed to 1 ml/well culture media. The next day, cytosolic Ca^{2 +} was imaged in cells transiently transfected with jGCaMP7s through a Zeiss LSM 800 confocal microscope. Dynamic intracellular Ca^{2 +} influx in response to Tm/Tg-induced ER stress was measured every 10 seconds for 100 seconds before Tm/Tg treatment to acquire baseline fluorescence (F0) and for 400 seconds after Tm/Tg administration to acquire F1. ΔF=F1−F0. Data were analyzed with ImageJ.

In vivo RGC Ca^{2 +} imaging with SLO. The mice were intravitreally injected with AAV2-mSncg-jGCaMP7s (9×10⁹ vg/retina) 4 weeks before imaging. The mice were anesthetized by xylazine and ketamine after dark adaptation for 30 minutes. Mydriasis was achieved by applying a drop of 1% tropicamide solution and a drop of 2.5% phenylephrine hydrochloride solution, which prevents pupillary contraction during recording. Right after ON crush, the mice were placed on a 3D-printed mouse holder with a 37° C. heater, and a custom-made+10D mouse contact lens (3.0 mm diameter, 1.6 mm BC, PMMA clear, Advanced Vision Technologies) attached to keep the cornea from drying. The retinal fundus was imaged by the Heidelberg Spectralis SLO/OCT system (Heidelberg Engineering, Germany) with a 55° lens using the fluorescein angiography scanning mode under the same sensitivity (sensitivity 75-85) and high-resolution (1536×1536 pixels).

Ex vivo ER Ca^{2 +} imaging in retina explants. ER calcium levels were measured using the Forster resonance energy transfer (FRET)-based ER targeted calcium sensor, D4ER. Mouse received intravitreal injection of AAV2-mSncg-D4ER to express D4ER in RGCs in vivo 4 weeks before ON crush injury, as well as intravitreal injection of DMSO (vehicle) or maprotiline compound. For imaging, retinas were dissected out at 3, 7 or 14dpc and plated onto laminin (Sigma, L2020) and poly-D-lysin-coated glass bottom dishes (MatTek, P35GC1.510C) and maintained in Neurobasal-A medium (ThermoFisher Scientific, 10888022) supplemented with L-glutamine (Gibco, 25030-081), penicillin/streptomycin (Gibco, 15140122) and B-27 (ThermoFisher Scientific, 0080085SA). Retina explants were imaged using a Zeiss LSM 800 microscope. ER [Ca^{2 +}] levels were determined by exciting D4ER at 440 nm to record the emitted light at 465-485 nm and 530-550 nm, and analyzed with ImageJ.

RGC counting. Whole-mount retinas were immunostained with the RBPMS antibody, 6-8 fields randomly sampled from peripheral regions of each retina using a 40× lens with a Zeiss M2 epifluorescence microscope, and RBPMS+RGCs counted by Volocity software (Quorum Technologies). The percentage of RGC survival was calculated as the ratio of surviving RGC numbers in injured eyes compared to contralateral uninjured eyes. The investigators who counted the cells were masked to the treatment of the samples.

ON semi-thin sections and quantification of surviving axons. ONs were post-fixed in situ with 2% glutaraldehyde and 2% PFA in 0.1 M PBS. Semi-thin (1 μm) cross sections of the ON 2 mm distal to the eye (globe) were collected. The sections were stained with 1% PPD for 0.5 hour before washing with methanol:isopropanol (1:1) 3 times×10 min and then mounted with Fluoromount-G. Four sections of each ON were imaged through a 100× lens of a Zeiss M2 epifluorescence microscope to cover the entire area of the ON without overlap. Two areas of 21.4 μm×29.1 μm were cropped from the center of each image, and the surviving axons within the designated areas counted manually using ImageJ. After counting all the images taken from a single nerve, the mean of the surviving axon number was calculated for each ON. The mean of the surviving axon number in the injured ON was compared to that in the contralateral control ON to yield a percentage of axon survival value.

Transmission Electron Microscope (TEM) Imaging and Quantification of Surviving Axons in ON Ultrathin Cross Sections. 70 nm ultrathin sections were collected onto formvar coated copper grids and dried overnight. Sections were then stained with uranyl acetate for 30 minutes, washed in PBS and then stained with lead citrate for 7 minutes. Sections were again washed and dried before observing under TEM. The cross-sections of the entire ON were examined and imaged randomly without overlap at 4000× with 11.6 μm×11.6 μm frames on a JEOL JEM-1400 TEM microscope (JEOL USA, Inc., Peabody, MA). For each ON, 25-45 images were taken to cover the whole area of the ON. Axons were counted manually with ImageJ's Cell Counter plugin.

CTB tracing in wholemount ON and imaging. Intravitreal injection of CTB was performed 48 hours before perfusion of the animals with 4% PFA in PBS. The ONs were carefully dissected with fine forceps and scissors and cleared with a modified iDISCO method: wash with PBS for 4×30 minutes; then immersed in a series of 20%, 40%, 60%, 80%, and 100% methanol in PBS for 30 minutes at each concentration; dichloromethane (DCM)/methanol (2:1) for 30 minutes; 100% DCM for 30 minutes and dibenzyl ether (DBE) for 10 minutes before mounting on slides. Tiled images of the wholemount ON were captured and stitched by a Zeiss LSM 880 confocal laser scanning microscope with 40×/1.0 Oil DIC (Carl Zeiss Microscopy, Thornwood, NY, USA). Positive CTB areas were identified based on a fluorescence intensity greater than the baseline intensity threshold. The percentage of the CTB positive area in the optic nerve was measured by NIH ImageJ.

Fluorescent in situ hybridization (ISH) of retina cross sections. Fluorescent in situ hybridization (FISH) was performed by using the RNAscope Multiplex Fluorescent Detection Reagents V2 (Advanced Cell Diagnostics, ACD, Hayward, CA, USA) according to the manufacturer's instructions. RNAscope probe Mm-Hrh1 (491141) was purchased from ACD. Adult mice were perfused with ice-cold 4% PFA/PBS, and eyes were dissected out and fixed in 4% PFA/PBS at 4° C. overnight. The eyes were dehydrated with increasing concentrations of sucrose solution (10%, 20% and 30%) overnight before embedding in OCT on dry ice. Serial cross sections (12 μm) were cut with a Leica cryostat and collected on Superfrost Plus Slides. The sections were pretreated with protease and then subjected to in situ hybridization with RNAscope Multiplex Fluorescent Detection Reagents V2 according to the manufacturer's instruction (Advanced Cell Diagnostics, Hayward, CA). Briefly, sections were hybridized with the probe solution, followed by amplification and probe detection using TSA plus fluorophores (AKOYA, Marlborough, MA, USA). The sections were mounted with Fluoromount-G (SouthernBiotech, Birmingham, AL, USA). Images were captured by a Zeiss LSM 880 confocal laser scanning microscope with 40×/1.0 Oil DIC (Carl Zeiss Microscopy, Thornwood, NY, USA).

Spectral-Domain Optical Coherence Tomography (SD-OCT) imaging. The mouse retina was scanned by the Heidelberg Spectralis SLO/OCT system (Heidelberg Engineering, Germany) with the ring scan mode centered by the ON head under high-resolution mode (each B-scan consisted of 1536 A scans). The ganglion cell complex (GCC) includes retinal nerve fiber layer (RNFL), ganglion cell layer (GCL) and inner plexiform layer (IPL). The average thickness of GCC around the ON head was measured manually with the aid of Heidelberg software.

Pattern Electroretinogram (PERG) recording. After anesthetization and pupil dilation, PERG of both eyes was recorded simultaneously with the Miami PERG system (Intelligent Hearing Systems, Miami, Florida) according to manufacturer's instructions. Two consecutive recordings of 200 traces were averaged to achieve one readout; each trace recorded up to 1020 ms. The first positive peak in the waveform was designated as P1 and the second negative peak as N2. The amplitude was measured from P1 to N2.

Optokinetic Tracking Response (OKR). Mice were placed on a platform in the center of four 17-inch LCD computer monitors (Dell, Phoenix, AZ), with a video camera above the platform to capture the movement of the mouse. A rotating cylinder with vertical sine wave grating was computed and projected to the four monitors by OptoMotry software (Cere-bralMechanics Inc, Lethbridge, Alberta, Canada). The sine wave grating, settled at 100% contrast and speed of 12 degrees per second, provides a virtual-reality environment to measure the spatial acuity (cycle/degree) of the left eye when rotated clockwise and the right eye when rotated counterclockwise. The maximum frequency (cycle/degree) that the mouse could track was identified and recorded by investigators masked to treatment. The relative percentages of visual acuity were calculated as the ratio of maximum frequency in disease eye compared to contralateral control eye.

Statistical Analysis. GraphPad Prism 7 was used to generate graphs and for statistical analyses. Data are presented as means±s.e.m. Student's t-test was used for two groups comparison and One-way ANOVA with post hoc test was used for multiple comparisons.

### REFERENCES

Tham Y C, Li X, Wong T Y, Quigley H A, Aung T, Cheng C Y. Global prevalence of glaucoma and projections of glaucoma burden through 2040: a systematic review and meta-analysis. Ophthalmology 121, 2081-2090 (2014).
Varma R, Lee P P, Goldberg I, Kotak S. An assessment of the health and economic burdens of glaucoma. American journal of ophthalmology 152, 515-522 (2011).
Nickells R W, Howell G R, Soto I, John S W. Under pressure: cellular and molecular responses during glaucoma, a common neurodegeneration with axonopathy. Annu Rev Neurosci 35, 153-179 (2012).
Jonas J B, Aung T, Bourne R R, Bron A M, Ritch R, Panda-Jonas S. Glaucoma. Lancet 390, 2183-2193 (2017).
Weinreb R N, et al. Primary open-angle glaucoma. Nat Rev Dis Primers 2, 16067 (2016).
Calkins D J. Adaptive responses to neurodegenerative stress in glaucoma. Prog Retin Eye Res, 100953 (2021).
Beykin G, Norcia A M, Srinivasan V J, Dubra A, Goldberg J L. Discovery and clinical translation of novel glaucoma biomarkers. Prog Retin Eye Res 80, 100875 (2021).
lyer J, Vianna J R, Chauhan B C, Quigley H A. Toward a new definition of glaucomatous optic neuropathy for clinical research. Curr Opin Ophthalmol 31, 85-90 (2020).
Stowell C, Burgoyne C F, Tamm E R, Ethier C R, Lasker IloA, Glaucomatous Neurodegeneration P. Biomechanical aspects of axonal damage in glaucoma: A brief review. Experimental eye research 157, 13-19 (2017).
Heijl A, et al. Reduction of intraocular pressure and glaucoma progression: results from the Early Manifest Glaucoma Trial. Arch Ophthalmol 120, 1268-1279 (2002).
Garway-Heath D F, et al. Latanoprost for open-angle glaucoma (UKGTS): a randomised, multicentre, placebo-controlled trial. Lancet 385, 1295-1304 (2015).
Anderson D R, Normal Tension Glaucoma S. Collaborative normal tension glaucoma study. Curr Opin Ophthalmol 14, 86-90 (2003).
Wormald R, Virgili G, Azuara-Blanco A. Systematic reviews and randomised controlled trials on open angle glaucoma. Eye (Lond) 34, 161-167 (2020).
Hu Y, et al. Differential effects of unfolded protein response pathways on axon injury-induced death of retinal ganglion cells. Neuron 73, 445-452 (2012).
Li S, Yang L, Selzer M E, Hu Y. Neuronal endoplasmic reticulum stress in axon injury and neurodegeneration. Annals of neurology, (2013).
Hu Y. Axon injury induced endoplasmic reticulum stress and neurodegeneration. Neural regeneration research 11, 1557-1559 (2016).
Walter P, Ron D. The unfolded protein response: from stress pathway to homeostatic regulation. Science 334, 1081-1086 (2011).
Hetz C, Zhang K, Kaufman R J. Mechanisms, regulation and functions of the unfolded protein response. Nat Rev Mol Cell Biol 21, 421-438 (2020).
Han D, et al. IRE1alpha kinase activation modes control alternate endoribonuclease outputs to determine divergent cell fates. Cell 138, 562-575 (2009).
Costa-Mattioli M, Walter P. The integrated stress response: From mechanism to disease. Science 368, (2020).
Han J, et al. ER-stress-induced transcriptional regulation increases protein synthesis leading to cell death. Nat Cell Biol 15, 481-490 (2013).
Hetz C, Axten J M, Patterson J B. Pharmacological targeting of the unfolded protein response for disease intervention. Nat Chem Biol 15, 764-775 (2019).
Yang L, et al. Rescue of Glaucomatous Neurodegeneration by Differentially Modulating Neuronal Endoplasmic Reticulum Stress Molecules. J Neurosci 36, 5891-5903 (2016).
Huang H, et al. Neuroprotection by eIF2alpha-CHOP inhibition and XBP-1 activation in EAE/optic neuritiss. Cell death & disease 8, e2936 (2017).
Wang Q, et al. Mouse gamma-Synuclein Promoter-Mediated Gene Expression and Editing in Mammalian Retinal Ganglion Cells. J Neurosci 40, 3896-3914 (2020).
Inglese J, et al. Quantitative high-throughput screening: a titration-based approach that efficiently identifies biological activities in large chemical libraries. Proc Natl Acad Sci USA 103, 11473-11478 (2006).
Sidrauski C, et al. Pharmacological brake-release of mRNA translation enhances cognitive memory. eLife 2, e00498 (2013).
Halliday M, et al. Repurposed drugs targeting eIF2&alpha;-P-mediated translational repression prevent neurodegeneration in mice. Brain 140, 1768-1783 (2017).
Urano F, et al. Coupling of stress in the ER to activation of JNK protein kinases by transmembrane protein kinase IRE1. Science 287, 664-666 (2000).
Fernandes K A, et al. JNK2 and JNK3 are major regulators of axonal injury-induced retinal ganglion cell death. Neurobiology of disease 46, 393-401 (2012).
Huang H, et al. AKT-dependent and -independent pathways mediate PTEN deletion-induced CNS axon regeneration. Cell death & disease 10, 203 (2019).
Yang L, et al. The mTORC1 effectors S6K1 and 4E-BP play different roles in CNS axon regeneration. Nature communications 5, 5416 (2014).
Miao L, Yang L, Huang H, Liang F, Ling C, Hu Y. mTORC1 is necessary but mTORC2 and GSK3beta are inhibitory for AKT3-induced axon regeneration in the central nervous system. eLife 5, e14908 (2016).
Zhang J, et al. Silicone oil-induced ocular hypertension and glaucomatous neurodegeneration in mouse. eLife 8, (2019).
Li L, Huang H, Fang F, Liu L, Sun Y, Hu Y. Longitudinal Morphological and Functional Assessment of RGC Neurodegeneration After Optic Nerve Crush in Mouse. Frontiers in cellular neuroscience 14, 109 (2020).
Zhang Y, et al. In vivo evaluation of retinal ganglion cells and optic nerve's integrity in large animals by multi-modality analysis. Experimental eye research 197, 108117 (2020).
Fang F, et al. Chronic mild and acute severe glaucomatous neurodegeneration derived from silicone oil-induced ocular hypertension. Scientific reports 11, 9052 (2021).
Zhang J, et al. A Reversible Silicon Oil-Induced Ocular Hypertension Model in Mice. Journal of visualized experiments: JoVE 153, (2019).
Prusky G T, Alam N M, Beekman S, Douglas R M. Rapid quantification of adult and developing mouse spatial vision using a virtual optomotor system. Invest Ophthalmol Vis Sci 45, 4611-4616 (2004).
Douglas R M, Alam N M, Silver B D, McGill T J, Tschetter W W, Prusky G T. Independent visual threshold measurements in the two eyes of freely moving rats and mice using a virtual-reality optokinetic system. Vis Neurosci 22, 677-684 (2005).
Porciatti V. Electrophysiological assessment of retinal ganglion cell function. Experimental eye research 141, 164-170 (2015).
Canonica G W, Blaiss M. Antihistaminic, anti-inflammatory, and antiallergic properties of the nonsedating second-generation antihistamine desloratadine: a review of the evidence. World Allergy Organ J 4, 47-53 (2011).
Kanba S, Richelson E. Histamine H1 receptors in human brain labelled with [3H]doxepin. Brain Res 304, 1-7 (1984).
von Coburg Y, Kottke T, Weizel L, Ligneau X, Stark H. Potential utility of histamine H3 receptor antagonist pharmacophore in antipsychotics. Bioorganic & medicinal chemistry letters 19, 538-542 (2009).
Appl H, Holzammer T, Dove S, Haen E, Strasser A, Seifert R. Interactions of recombinant human histamine H (1)R, H (2)R, H (3)R, and H (4) R receptors with 34 antidepressants and antipsychotics. Naunyn Schmiedebergs Arch Pharmacol 385, 145-170 (2012).
Papandreou I, et al. Identification of an Ire1alpha endonuclease specific inhibitor with cytotoxic activity against human multiple myeloma. Blood 117, 1311-1314 (2011).
Panula P, et al. International Union of Basic and Clinical Pharmacology. XCVIII. Histamine Receptors. Pharmacol Rev 67, 601-655 (2015).
Knoferle J, et al. Mechanisms of acute axonal degeneration in the optic nerve in vivo. Proc Natl Acad Sci USA 107, 6064-6069 (2010).
Ribas V T, Koch J C, Michel U, Bahr M, Lingor P. Attenuation of Axonal Degeneration by Calcium Channel Inhibitors Improves Retinal Ganglion Cell Survival and Regeneration After Optic Nerve Crush. Mol Neurobiol 54, 72-86 (2017).
Stirling D P, Cummins K, Wayne Chen S R, Stys P. Axoplasmic reticulum Ca (2+) release causes secondary degeneration of spinal axons. Annals of neurology 75, 220-229 (2014).
Villegas R, et al. Calcium release from intra-axonal endoplasmic reticulum leads to axon degeneration through mitochondrial dysfunction. J Neurosci 34, 7179-7189 (2014).
Orem B C, Rajaee A, Stirling D P. IP3R-mediated intra-axonal Ca (2+) release contributes to secondary axonal degeneration following contusive spinal cord injury. Neurobiology of disease 146, 105123 (2020).
Dana H, et al. High-performance calcium sensors for imaging activity in neuronal populations and microcompartments. Nature methods 16, 649-657 (2019).
Greotti E, Wong A, Pozzan T, Pendin D, Pizzo P. Characterization of the ER-Targeted Low Affinity Ca (2+) Probe D4ER. Sensors (Basel) 16, (2016).
Kipanyula M J, et al. Ca^{2 +} dysregulation in neurons from transgenic mice expressing mutant presenilin 2. Aging Cell 11, 885-893 (2012).
Lauterbach E C. Neuroprotective effects of psychotropic drugs in Huntington's disease. Int J Mol Sci 14, 22558-22603 (2013).
Grandjean J M D, et al. Pharmacologic IRE1/XBP1s activation confers targeted ER proteostasis reprogramming. Nat Chem Biol 16, 1052-1061 (2020).
Plate L, et al. Small molecule proteostasis regulators that reprogram the ER to reduce extracellular protein aggregation. eLife 5, (2016).
Bruhat A, Jousse C, Carraro V, Reimold A M, Ferrara M, Fafournoux P. Amino acids control mammalian gene transcription: activating transcription factor 2 is essential for the amino acid responsiveness of the CHOP promoter. Mol Cell Biol 20, 7192-7204 (2000).
Yamazaki T, et al. Regulation of the human CHOP gene promoter by the stress response transcription factor ATF5 via the AARE1 site in human hepatoma HepG2 cells. Life sciences 87, 294-301 (2010).
Renier N, Wu Z, Simon D J, Yang J, Ariel P, Tessier-Lavigne M. iDISCO: a simple, rapid method to immunolabel large tissue samples for volume imaging. Cell 159, 896-910 (2014).

Sequences

(SEQ ID NO: 31) mSncg-1.45kb promoter
ccattgacgtcaataatgacgtatgttcccatagtaacgccaatagggactttccattgacgtcaatgggtggagtatttacggtaaact
gcccacttggcagtacatcaagtgtatcatatgggtcccatgccactagtgggagctgtgttacctgttgcagccccacccaaagcccc
tgctataggtcaagcaggaatcaccctgccatccccagcctggggcctggagtaccagatccaggaaactagcatcccttagctata
gagatagccacacatcagcccattcctcagatgtgtatctggggctcagacatcatctcccgatctccgacaagggcaggatttcctta
ccgtctgatggggtctctgctggtatcctcagcccctagtctccagccttcaggcatgccgggcctattgagatgggagaacttggtacc
ggggtcctgtgcccaggaccctagcagtccccagctcaggtacaccccaaagcccagcagcagtgtcgggatcatggtgagggg
ctcctgtgctgatgctcagccttacaagtgactctcaaatttgctggtgatgtggtcttcaagcgaaatgtcagaaagaaaagaaaaca
cgaggacaacaaagggaggaagtggcctggtccggcccacccggcaagtctcatccgcccccgcccccgccccttccagcctgg
cccccttggaggcctccaaccactcaggtcaattcctgtgtcctgagggcacttgaatcagggacacgggatttggtagacacataaa
ggtggccccattaaacttatttctccaggactctgtcgtgggcctggaggagatctggtgccacccatactgttggccaggaagtgggg
aacgggcacatcacacctgctcggcaccttgggctatgggaactagcaggtgggtgggaactcagagaaggaaagggactatgct
agaatcacacagcgggcagcccagtctagggcatggggagcagctttgggtgtttctggcctcagccttccaacaggtttggctaga
gctccaggctcaagagcatccaggatacagtggggaactggataacagggcagcctgcaggttggccattcattggattggccctg
accccggcccagcctgggacactgaggcatcatcagtcaaggcacttttcttctgcatataagagccagggcacgagaccaccag
ggctttccaaggatgaatgaggtgtaatgatagattaggatatgtccagcctccaacacgctctccctcccccagggccaacaagagt
cagcagggcagaatagagccagtaggggcccgggccctgctcgctggtatccccgtgaggcatgccttctctctggcccgccctcc
ctgcccccaccctggcccgggctggctgggctccagccagcagccacagcatcaatatttcatctgcgtcaataagaggcagtagc
agcagagacagcggctgcggcagcactccagtccatagcttgcagcagccaggttccatccttgcaaacaccatggacgtcttcaa
g
(SEQ ID NO: 32) mSncg-1.03b promoter
ccattgacgtcaataatgacgtatgttcccatagtaacgccaatagggactttccattgacgtcaatgggtggagtatttacggtaaact
gcccacttggcagtacatcaagtgtatcatatgtcagccttacaagtgactctcaaatttgctggtgatgtggtcttcaagcgaaatgtca
gaaagaaaagaaaacacgaggacaacaaagggaggaagtggcctggtccggcccacccggcaagtctcatccgcccccgcc
cccgccccttccagcctggcccccttggaggcctccaaccactcaggtcaattcctgtgtcctgagggcacttgaatcagggacacgg
gatttggtagacacataaaggtggccccattaaacttatttctccaggactctgtcgtgggcctggaggagatctggtgccacccatact
gttggccaggaagtggggaacgggcacatcacacctgctcggcaccttgggctatgggaactagcaggtgggtgggaactcagag
aaggaaagggactatgctagaatcacacagcgggcagcccagtctagggcatggggagcagctttgggtgtttctggcctcagcctt
ccaacaggtttggctagagctccaggctcaagagcatccaggatacagtggggaactggataacagggcagcctgcaggttggcc
attcattggattggccctgaccccggcccagcctgggacactgaggcatcatcagtcaaggcacttttcttctgcatataagagccagg
gcacgagaccaccagggctttccaaggatgaatgaggtgtaatgatagattaggatatgtccagcctccaacacgctctccctcccc
cagggccaacaagagtcagcagggcagaatagagccagtaggggcccgggccctgctcgctggtatccccgtgaggcatgcctt
ctctctggcccgccctccctgcccccaccctggcccgggctggctgggctccagccagcagccacagcatcaatatttcatctgcgtc
aataagaggcagtagcagcagagacagcggctgcggcagcactccagtccatagcttgcagcagccaggttccatccttgcaaac
accatggacgtcttcaag
(SEQ ID NO: 33) mSncg-0.66kb promoter
ccattgacgtcaataatgacgtatgttcccatagtaacgccaatagggactttccattgacgtcaatgggtggagtatttacggtaaact
gcccacttggcagtacatcaagtgtatcatatgctatgggaactagcaggtgggtgggaactcagagaaggaaagggactatgcta
gaatcacacagcgggcagcccagtctagggcatggggagcagctttgggtgtttctggcctcagccttccaacaggtttggctagag
ctccaggctcaagagcatccaggatacagtggggaactggataacagggcagcctgcaggttggccattcattggattggccctga
ccccggcccagcctgggacactgaggcatcatcagtcaaggcacttttcttctgcatataagagccagggcacgagaccaccagg
gctttccaaggatgaatgaggtgtaatgatagattaggatatgtccagcctccaacacgctctccctcccccagggccaacaagagtc
agcagggcagaatagagccagtaggggcccgggccctgctcgctggtatccccgtgaggcatgccttctctctggcccgccctccct
gcccccaccctggcccgggctggctgggctccagccagcagccacagcatcaatatttcatctgcgtcaataagaggcagtagcag
cagagacagcggctgcggcagcactccagtccatagcttgcagcagccaggttccatccttgcaaacaccatggacgtcttcaag
(SEQ ID NO: 34) mSncg-0.27kb promoter
ccattgacgtcaataatgacgtatgttcccatagtaacgccaatagggactttccattgacgtcaatgggtggagtatttacggtaaact
gcccacttggcagtacatcaagtgtatcatatgaagagtcagcagggcagaatagagccagtaggggcccgggccctgctcgctg
gtatccccgtgaggcatgccttctctctggcccgccctccctgcccccaccctggcccgggctggctgggctccagccagcagccac
agcatcaatatttcatctgcgtcaataagaggcagtagcagcagagacagcggctgcggcagcactccagtccatagcttgcagca
gccaggttccatccttgcaaacaccatggacgtcttcaag

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112 (f) or 35 U.S.C. § 112 (6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112 (6) is not invoked.

1. A method of treating an optic nerve (ON) neuropathy in a mammalian subject in need thereof, the method comprising:
administering an effective dose of an HRH1 antagonist to the optic nerve of the subject, thereby treating the ON neuropathy.

2. The method of claim 1, wherein the administration of an HRH1 antagonist reduces or ameliorates degeneration of axons and/or soma of retinal ganglion cells (RGCs).

3. The method of claim 1, wherein the composition is administered intravitreally.

4. The method of claim 1, wherein the composition administered systemically.

5. The method of claim 1, wherein the ON neuropathy is retinal ganglion cell degeneration, including glaucoma, optic neuritis, ON traumatic injury and other ON-related diseases.

6. The method of claim 1, wherein the ON neuropathy is glaucoma.

7. The method of claim 1, wherein the subject is human.

8. The method of claim 1, wherein the HRH1 antagonist is a tricyclic compound.

9. The method of claim 8, wherein the tricyclic compound is selected from maprotiline, amoxapine, desipramine, desloratadine, trifluoperazine, clomipramine, amitriptyline, quetiapine, olanzapine, doxepin, loxapine, integrated stress response inhibitor (ISRIB), and norquetiapine.

10. The method of claim 8, wherein the tricyclic agent is maprotiline formulated for ocular delivery.

11. The method of claim 1, wherein the HRH1 antagonist comprises an AAV vector, comprising:
a murine γ-synuclein promoter in operable linkage with a nucleic acid encoding a Cas9 nuclease; and
a U6 promoter in operable linkage with at least one guide (gRNA) 20-21 nucleotides in length, wherein each gRNA precedes an −NGG protospacer, and wherein each gRNA targets histamine receptor H1 (HRH1).

12. The method of claim 11, wherein the murine γ-synuclein promoter is selected from the sequence of SEQ ID NO:3, 4, 5, 6, 7, or a variant thereof.


SEQUENCE LISTING (34 sequences)

SEQ ID NO: 1 - DNA, 20 bp, synthetic construct
  source 1..20: mol_type = other DNA
ggggcttggtatatatgtgg

SEQ ID NO: 2 - DNA, 20 bp, synthetic construct
  source 1..20: mol_type = other DNA
ccttgtagttgagaaccagg

SEQ ID NO: 3 - DNA, 22 bp, synthetic construct
  source 1..22: mol_type = other DNA
gtctcctctgacttcaacagcg

SEQ ID NO: 4 - DNA, 22 bp, synthetic construct
  source 1..22: mol_type = other DNA
accaccctgttgctgtagccaa

SEQ ID NO: 5 - DNA, 20 bp, synthetic construct
  source 1..20: mol_type = other DNA
tggaagcagcaaatgagacg

SEQ ID NO: 6 - DNA, 20 bp, synthetic construct
  source 1..20: mol_type = other DNA
tgaggaggctggagaaagtg

SEQ ID NO: 7 - DNA, 20 bp, synthetic construct
  source 1..20: mol_type = other DNA
cgaacgtgatccgctacttc

SEQ ID NO: 8 - DNA, 20 bp, synthetic construct
  source 1..20: mol_type = other DNA
atgttgagggagtggaggtg

SEQ ID NO: 9 - DNA, 23 bp, synthetic construct
  source 1..23: mol_type = other DNA
gtcccaaggctttggaatctgtc

SEQ ID NO: 10 - DNA, 22 bp, synthetic construct
  source 1..22: mol_type = other DNA
cctaccaagacaggagttctgg

SEQ ID NO: 11 - DNA, 22 bp, synthetic construct
  source 1..22: mol_type = other DNA
accaagggagaaccaggaaacg

SEQ ID NO: 12 - DNA, 22 bp, synthetic construct
  source 1..22: mol_type = other DNA
tcaccattcggtcaatcagagc

SEQ ID NO: 13 - DNA, 20 bp, synthetic construct
  source 1..20: mol_type = other DNA
gtccctccaacaacagcaag

SEQ ID NO: 14 - DNA, 20 bp, synthetic construct
  source 1..20: mol_type = other DNA
tgtcatccaacgtggtcaga

SEQ ID NO: 15 - DNA, 20 bp, synthetic construct
  source 1..20: mol_type = other DNA
gcctgcatctgctttatgct

SEQ ID NO: 16 - DNA, 20 bp, synthetic construct
  source 1..20: mol_type = other DNA
tctgcaaggctgtgaagaga

SEQ ID NO: 17 - DNA, 20 bp, synthetic construct
  source 1..20: mol_type = other DNA
ggaggaagtgctcagcaaag

SEQ ID NO: 18 - DNA, 20 bp, synthetic construct
  source 1..20: mol_type = other DNA
acatctctgtcgtcgtcctc

SEQ ID NO: 19 - DNA, 20 bp, synthetic construct
  source 1..20: mol_type = other DNA
ggaaggaggagcgctaggtc

SEQ ID NO: 20 - DNA, 20 bp, synthetic construct
  source 1..20: mol_type = other DNA
atcctgcaccctccgactac

SEQ ID NO: 21 - DNA, 20 bp, synthetic construct
  source 1..20: mol_type = other DNA
cgatgatcccacagactcca

SEQ ID NO: 22 - DNA, 20 bp, synthetic construct
  source 1..20: mol_type = other DNA
ccgtccatctcttcatccca

SEQ ID NO: 23 - DNA, 21 bp, synthetic construct
  source 1..21: mol_type = other DNA
gcctgtatttctagacctgcc

SEQ ID NO: 24 - DNA, 20 bp, synthetic construct
  source 1..20: mol_type = other DNA
ttcatcttgccagccagttg

SEQ ID NO: 25 - DNA, 20 bp, synthetic construct
  source 1..20: mol_type = other DNA
ctccagagacggagtccaag

SEQ ID NO: 26 - DNA, 16 bp, synthetic construct
  source 1..16: mol_type = other DNA
cacctgctgcggactc

SEQ ID NO: 27 - DNA, 21 bp, synthetic construct
  source 1..21: mol_type = other DNA
gtctccgtcctccttaacccc

SEQ ID NO: 28 - DNA, 20 bp, synthetic construct
  source 1..20: mol_type = other DNA
gattctccgtcctccttaac

SEQ ID NO: 29 - protein, 487 aa, Homo sapiens
MSLPNSSCLLEDKMCEGNKTTMASPQLMPLVVVLSTICLVTVGLNLLVLYAVRSERKLHTVGNLYIVSLSVADLIVGAVVMPMNILYLLMSKWSLGRPLCLFWLSMDYVASTASIFSVFILCIDRYRSVQQPLRYLKYRTKTRASATILGAWFLSFLWVIPILGWNHFMQQTSVRREDKCETDFYDVTWFKVMTAIINFYLPTLLMLWFYAKIYKAVRQHCQHRELINRSLPSFSEIKLRPENPKGDAKKPGKESPWEVLKRKPKDAGGGSVLKSPSQTPKEMKSPVVFSQEDDREVDKLYCFPLDIVHMQAAAEGSSRDYVAVNRSHGQLKTDEQGLNTHGASEISEDQMLGDSQSFSRTDSDTTTETAPGKGKLRSGSNTGLDYIKFTWKRLRSHSRQYVSGLHMNRERKAAKQLGFIMAAFILCWIPYFIFFMVIAFCKNCCNEHLHMFTIWLGYINSTLNPLIYPLCNENFKKTFKRILHIRS

SEQ ID NO: 30 - protein, 488 aa, Mus musculus
MSLPNTSSASEDKMCEGNRTAMASPQLLPLVVVLSSISLVTVGLNLLVLYAVRSERKLHTVGNLYIVSLSVADLIVGAVVMPMNILYLIMTKWSLGRPLCLFWLSMDYVASTASIFSVFILCIDRYRSVQQPLRYLRYRTKTRASATILGAWFLSFLWVIPILGWHHFTPLAPELREDKCETDFYNVTWFKIMTAIINFYLPTLLMLWFYVKIYKAVRRHCQHRQLTNGSLPTFLEIKLRSEDAKEGAKKPGKESPWGVQKRPSRDPTGGLDQKSTSEDPKVTSPTVFSQEGERETVTRPCFRLDVMQTQPVPEGDARGSKANDQTLSQPKMDEQSLSTCRRISETSEDQTLVDRQSFSRTTDSDTSIEPGLGKVKARSRSNSGLDYIKVTWKRLRSHSRQYVSGLHLNRERKAAKQLGCIMAAFILCWIPYFIFFMVIAFCNSCCSEPVHMFTIWLGYINSTLNPLIYPLCNENFKKTFKKILHIRS

SEQ ID NO: 31 - DNA, 1569 bp, Mus musculus
  source 1..1569: mol_type = genomic DNA
ccattgacgtcaataatgacgtatgttcccatagtaacgccaatagggactttccattgacgtcaatgggtggagtatttacggtaaactgcccacttggcagtacatcaagtgtatcatatgggtcccatgccactagtgggagctgtgttacctgttgcagccccacccaaagcccctgctataggtcaagcaggaatcaccctgccatccccagcctggggcctggagtaccagatccaggaaactagcatcccttagctatagagatagccacacatcagcccattcctcagatgtgtatctggggctcagacatcatctcccgatctccgacaagggcaggatttccttaccgtctgatggggtctctgctggtatcctcagcccctagtctccagccttcaggcatgccgggcctattgagatgggagaacttggtaccggggtcctgtgcccaggaccctagcagtccccagctcaggtacaccccaaagcccagcagcagtgtcgggatcatggtgaggggctcctgtgctgatgctcagccttacaagtgactctcaaatttgctggtgatgtggtcttcaagcgaaatgtcagaaagaaaagaaaacacgaggacaacaaagggaggaagtggcctggtccggcccacccggcaagtctcatccgcccccgcccccgccccttccagcctggcccccttggaggcctccaaccactcaggtcaattcctgtgtcctgagggcacttgaatcagggacacgggatttggtagacacataaaggtggccccattaaacttatttctccaggactctgtcgtgggcctggaggagatctggtgccacccatactgttggccaggaagtggggaacgggcacatcacacctgctcggcaccttgggctatgggaactagcaggtgggtgggaactcagagaaggaaagggactatgctagaatcacacagcgggcagcccagtctagggcatggggagcagctttgggtgtttctggcctcagccttccaacaggtttggctagagctccaggctcaagagcatccaggatacagtggggaactggataacagggcagcctgcaggttggccattcattggattggccctgaccccggcccagcctgggacactgaggcatcatcagtcaaggcacttttcttctgcatataagagccagggcacgagaccaccagggctttccaaggatgaatgaggtgtaatgatagattaggatatgtccagcctccaacacgctctccctcccccagggccaacaagagtcagcagggcagaatagagccagtaggggcccgggccctgctcgctggtatccccgtgaggcatgccttctctctggcccgccctccctgcccccaccctggcccgggctggctgggctccagccagcagccacagcatcaatatttcatctgcgtcaataagaggcagtagcagcagagacagcggctgcggcagcactccagtccatagcttgcagcagccaggttccatccttgcaaacaccatggacgtcttcaag

SEQ ID NO: 32 - DNA, 1148 bp, Mus musculus
  source 1..1148: mol_type = genomic DNA
ccattgacgtcaataatgacgtatgttcccatagtaacgccaatagggactttccattgacgtcaatgggtggagtatttacggtaaactgcccacttggcagtacatcaagtgtatcatatgtcagccttacaagtgactctcaaatttgctggtgatgtggtcttcaagcgaaatgtcagaaagaaaagaaaacacgaggacaacaaagggaggaagtggcctggtccggcccacccggcaagtctcatccgcccccgcccccgccccttccagcctggcccccttggaggcctccaaccactcaggtcaattcctgtgtcctgagggcacttgaatcagggacacgggatttggtagacacataaaggtggccccattaaacttatttctccaggactctgtcgtgggcctggaggagatctggtgccacccatactgttggccaggaagtggggaacgggcacatcacacctgctcggcaccttgggctatgggaactagcaggtgggtgggaactcagagaaggaaagggactatgctagaatcacacagcgggcagcccagtctagggcatggggagcagctttgggtgtttctggcctcagccttccaacaggtttggctagagctccaggctcaagagcatccaggatacagtggggaactggataacagggcagcctgcaggttggccattcattggattggccctgaccccggcccagcctgggacactgaggcatcatcagtcaaggcacttttcttctgcatataagagccagggcacgagaccaccagggctttccaaggatgaatgaggtgtaatgatagattaggatatgtccagcctccaacacgctctccctcccccagggccaacaagagtcagcagggcagaatagagccagtaggggcccgggccctgctcgctggtatccccgtgaggcatgccttctctctggcccgccctccctgcccccaccctggcccgggctggctgggctccagccagcagccacagcatcaatatttcatctgcgtcaataagaggcagtagcagcagagacagcggctgcggcagcactccagtccatagcttgcagcagccaggttccatccttgcaaacaccatggacgtcttcaag

SEQ ID NO: 33 - DNA, 780 bp, Mus musculus
  source 1..780: mol_type = genomic DNA
ccattgacgtcaataatgacgtatgttcccatagtaacgccaatagggactttccattgacgtcaatgggtggagtatttacggtaaactgcccacttggcagtacatcaagtgtatcatatgctatgggaactagcaggtgggtgggaactcagagaaggaaagggactatgctagaatcacacagcgggcagcccagtctagggcatggggagcagctttgggtgtttctggcctcagccttccaacaggtttggctagagctccaggctcaagagcatccaggatacagtggggaactggataacagggcagcctgcaggttggccattcattggattggccctgaccccggcccagcctgggacactgaggcatcatcagtcaaggcacttttcttctgcatataagagccagggcacgagaccaccagggctttccaaggatgaatgaggtgtaatgatagattaggatatgtccagcctccaacacgctctccctcccccagggccaacaagagtcagcagggcagaatagagccagtaggggcccgggccctgctcgctggtatccccgtgaggcatgccttctctctggcccgccctccctgcccccaccctggcccgggctggctgggctccagccagcagccacagcatcaatatttcatctgcgtcaataagaggcagtagcagcagagacagcggctgcggcagcactccagtccatagcttgcagcagccaggttccatccttgcaaacaccatggacgtcttcaag

SEQ ID NO: 34 - DNA, 388 bp, Mus musculus
  source 1..388: mol_type = genomic DNA
ccattgacgtcaataatgacgtatgttcccatagtaacgccaatagggactttccattgacgtcaatgggtggagtatttacggtaaactgcccacttggcagtacatcaagtgtatcatatgaagagtcagcagggcagaatagagccagtaggggcccgggccctgctcgctggtatccccgtgaggcatgccttctctctggcccgccctccctgcccccaccctggcccgggctggctgggctccagccagcagccacagcatcaatatttcatctgcgtcaataagaggcagtagcagcagagacagcggctgcggcagcactccagtccatagcttgcagcagccaggttccatccttgcaaacaccatggacgtcttcaag